(19) United States
(12) Patent Application Publication
(10) Pub. No.: US 2025/0198993 A1
(43) Pub. Date: Jun. 19, 2025

(54) T LYMPHOCYTE ACTIVITY SCREENING AND SEQUENCING

(71) Applicant: THE CHILDREN'S HOSPITAL OF PHILADELPHIA, Philadelphia, PA (US)

(72) Inventors: Siwei LUO, Philadelphia, PA (US); Yi XING, Philadelphia, PA (US); Lan LIN, Philadelphia, PA (US)

(73) Assignee: THE CHILDREN'S HOSPITAL OF PHILADELPHIA, Philadelphia, PA (US)

(21) Appl. No.: 18/874,851

(22) PCT Filed: Jun. 7, 2023
(86) PCT No.: PCT/US2023/024722

§ 371 Date: Dec. 13, 2024

Related U.S. Application Data
(60) Provisional application No. 63/353,184, filed on Jun. 17, 2022.

(51) Int. Cl. G01N 33/52; C12N 5/0783; C12N 15/10; G01N 1/28; G01N 15/02; G01N 33/50

(52) U.S. Cl. CPC G01N 33/52; C12N 5/0636; C12N 15/1096; G01N 1/28; G01N 15/02; G01N 33/505; G01N 2015/0288

(57) ABSTRACT

The present disclosure is directed to the accurate and high throughput method for screening and identifying antigen-reactive T Cell Receptors (TCRs) capable of triggering effective T-cell activation.

### BACKGROUND

This application claims the benefit of priority to U.S. Provisional Application No. 63/353,184 filed Jun. 17, 2022, the entire contents of which are hereby incorporated by reference.

### REFERENCE TO A SEQUENCE LISTING

This application contains a Sequence Listing XML, which has been submitted electronically and is hereby incorporated by reference in its entirety. Said Sequence Listing XML, created on May 22, 2023, is named CHOPP0058WO.xml and is ˜136 kilobytes in size.

### 1. FIELD OF THE DISCLOSURE

The present disclosure relates generally to the fields of molecular biology and immunotherapy. More particularly, the disclosure relates to methods for the rapid and efficient identification of high-activation potential candidates for T Cell Receptor (TCR) therapy.

### 2. BACKGROUND

Cancer immunotherapy is a promising option for chemotherapy-resistant cancers and cancers with poor salvage rates for recurrent disease (Wedekind, Denton et al., 2018). Cancer immunotherapy strategies can be broadly categorized into immune checkpoint inhibitors that augment the suppressed immune response (Sharma and Allison, 2015), and adoptive cell therapy (ACT) strategies that direct or engineer the immune response against tumor-specific antigens. General ACTs include chimeric antigen receptor T-cell (CAR-T), and engineered T-cell receptor (TCR) therapies. CAR-T therapies are designed to directly target tumor- or lineage-specific cell-surface proteins by using artificial receptors introduced into immune effector cells (Hu, Ott et al., 2018). In contrast, TCR therapies use naturally occurring TCRs to target tumor-specific intracellular proteins that have been processed and presented on the tumor cell surface via the major histocompatibility complex (MHC) class I antigen presentation pathway. Because there are far more tumor-specific protein sequences within a cell that can be presented by the MHC than there are tumor-specific proteins on the cell surface (Tsimberidou, Van Morris et al., 2021), TCR therapies have a potentially broader applicability, especially in the treatment of solid tumors (Tsimberidou, Van Morris et al., 2021; Marofi, Motavalli et al., 2021).

In TCR therapies, TCR repertoire profiling and TCR selection have been the core component of the treatment development (Hogan, Courtier et al., 2019; Valpione, Mundra et al., 2021; Zhang, Xiong et al., 2021). Multiple fluorescence based TCR selection technologies are currently available. However, current methods all have critical limitations. For example, MHC multimer technologies use fluorescently labeled oligomeric MHC molecules formed through a tetrameric agent or dextran backbone to isolate and characterize antigen-specific T cell populations (Bentzen, Marquard et al., 2016; Chang, 2021). These MHC multimer assays focus on high-affinity physical/molecular interactions between the TCR and MHC-antigen (peptide-MHC/pMHC) complex. However, T-cell affinity from these assays is not equivalent to T-cell efficacy (Rius, Attaf et al., 2018), i.e., how effectively the TCR-pMHC interaction activates T cells (Stone and Kranz, 2013). As TCR efficacy is a crucial criterion for clinical applications of TCR therapy, there is a critical unmet need to develop a TCR selection technology that identifies TCRs based on their ability to activate the T-cells.

Cytokine secretion is one of the most important markers of T cell development and cytotoxic activities (Huse, Lillemeier et al., 2006; De Biasi, Meschiari et al., 2020; Mazet, Mahale et al., 2023). Recent efforts have been focusing on developing new methods to directly measure cytokine production capacity of T cells through intracellular cytokine staining (Nicolet, Guislain et al., 2017; Nesterenko, McLaughlin et al., 2021). However, these methods use fixed cells to measure intracellular RNA or protein products of cytokines rather than the actual secretion of cytokines from living cells. Additionally, T cells undergo complex intercellular communications and can receive signals from other T cells or PBMCs in conventional bulk activation assays, which could alter the behavior of specific T cells in such assays (Campbell, Foerster et al., 2011; Altan-Bonnet and Mukherjee 2019) or generate artificial immunosuppression of T cells (Gong, Li et al., 2022). A droplet-based T cell screening technology can potentially provide an enclosed environment for the accurate measurement of each individual T cell activation through cytokine secretion and prevent the cross talk among neighboring T cells (Yuan, Brouchon et al., 2020).

Improved methods for identifying useful TCR's are urgently needed.

### SUMMARY

Thus, in accordance with the present disclosure, there is provided a method detecting T Cell Receptor (TCR) activation on a target T cell comprising:
(a) providing a single T cell decorated with a cytokine-specific detectable aptamer beacon with a modification that allows said cytokine-specific detectable aptamer beacon to be immobilized to the T cell's surface;
(b) providing one or more antigen-loaded artificial antigen presenting cells (aAPCs);
(c) co-encapsulating said T cell and said one or more aAPCs in a microdroplet;
(d) incubating said microdroplet for a time sufficient to permit T cell activation by said aAPC;
(e) sorting and extracting an activated T cell, wherein said activated T cell is sorted by fluorescence activated cell sorting based on activation and detection of said cytokine-specific detectable label; and
(f) sequencing TCR sequences from the sorted and extracted T cell of step (e).
   The method may comprise multiple genetically distinct single T cells in distinct microdroplets processed together. Steps (c) and (d) may be performed on a microfluidic chip, such as a PDM or PMMA chip. The microdroplet may be generated through a T-junction configuration or flow-focusing configuration. Step (d) may comprise incubation at about 35-39° C. for about 24-72 hours. Step (f) may comprise a single-cell sequencing protocol, such as 10× Genomics protocol, inDrop protocol and SMART-seq protocol.

The cytokine-specific detectable aptamer beacon may be a single-stranded DNA (ssDNA) or an RNA oligonucleotide sequence that binds to its target cytokine with high specificity and affinity, such as nucleic acid aptamer with a fluorescent label that is quenched prior to binding to the cytokine for which the aptamer is specific. The cytokine for which the cytokine-specific detectable aptamer beacon may be specific is selected from IFN-γ, TNF-α, IL-6, Granzyme B and Perforin. The microdroplet may be a 100-200 μm diameter water-in-oil droplet. The aAPC may be a xenogeneic cell (such as K562-aAPC), PLGA microparticle, sepharose microparticle, polystyrene microparticle, liposome and nanoparticle. The modification may be streptavidin-aptamer conjugate bound to a cell surface amino group through NHS-biotin crosslinker or a lipophilic residue (cholesterol, tocopherol, C18 chains, diacyl phospholipid, etc.) modified aptamer displayed on cell surface by hydrophobic insertion.

The method may further comprise cloning a TCR gene from said T cell. The method may further comprise transforming a target cell with said TCR, such as with a non-viral or viral (e.g., lentiviral) vectors. The target cell may be a T cell circulating in a patients' peripheral blood.

Also provided is a T Cell Receptor identified according to a method as described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Aptamer-based T Lymphocyte Activity Screening and Sequencing (ATLAS-seq). (FIG. 1A) Stem-loop structure of IFNγ-specific aptamer beacon. The 5′ end of the aptamer is decorated by Cy3 fluorophore. The 3′ end of the aptamer is decorated by TAO quencher, iSP18 linker and cholesterol. (FIG. 1B) MART1 peptides loaded aAPCs were added to DMF5 Jurkat cells or control Jurkat cells decorated with IFNγ-specific aptamer beacon. Average cellular fluorescence intensities of the aptamer beacons were measured to detect IFNγ secretion due to MART1 antigen-specific Jurkat T cell activation. Error bars: standard deviation (SD), n=3. P-values were calculated by t-test. ***: p≤0.001. (FIG. 1C) Control Jurkat and DMF5 Jurkat cells were decorated with IFNγ-specific aptamer beacons and co-encapsulated in droplets with MART1 peptide-loaded aAPCs or empty aAPCs for 2 days at 37° C., 5% CO₂. Fluorescence intensities of aptamer beacons were measured by flow cytometry to show the IFNγ secretion from MART1 antigen-specific activated single Jurkat T cells in droplets. Aptamer beacon decorated control Jurkat cells: with empty aAPCs (green); with MART1 peptide-loaded aAPCs (orange); with aptamer cDNA (red). Aptamer beacon decorated DMF5 Jurkat cells: with empty aAPCs (gray); with MART1 peptide-loaded aAPCs (dark blue); with aptamer cDNA (light blue). Aptamer cDNA: DNA oligo with complementary sequence to the aptamer stem region. (FIG. 1D) ATLAS-seq workflow. Single aptamer labelled T cell is co-encapsulated with antigen peptide-loaded aAPC beads within uniformly sized water-in-oil microdroplets using a cross junction channel in microfluidic chip. Droplets are incubated off-chip to accumulate IFNγ secreted from the encapsulated single T cell. Secreted IFNγ will switch on the Cy3 fluorescence signal of the aptamer beacon on the T cell surface. After breaking the emulsion, recovered T cells are analyzed and sorted by flow cytometry based on the intensity of Cy3 fluorescence signal. Sorted T cells are used for preparing both single-cell TCR-seq and single-cell RNA-seq libraries using 10× Genomics Chromium Controller.

FIGS. 2A-G. CMV antigen-specific TCR profiling by Dextramer staining and ATLAS-seq. (FIG. 2A) Flow cytometry analysis for selecting CMV antigen-specific CD8⁺ T cells through Dextramer staining or ATLAS-seq. T cells from the same CMV positive donor human PBMCs were stimulated with aAPCs carrying the cognate peptide CMV pp65 HLA-A2 (NLVPMVATV) and subjected to either Dextramer staining or ATLAS-seq. CD8/PE (Dextramer staining) or CD8/Cy3 (ATLAS-seq) double positive cells were selected using each method for 10× Genomics single-cell sequencing. Input: unstimulated T cells from the same donor PBMCs. (FIG. 2B) Average proportions of the flow cytometry-selected TCR clonotypes with at least two cells from the two replicates of Dextramer staining or ATLAS-seq. Remaining panels (FIGS. 2C-G) are based on these flow cytometry-selected TCR clonotypes with at least two cells from the two replicates of Dextramer staining or ATLAS-seq. (FIG. 2C) Overlaps of the TCRα or TCRb clonotypes. (FIG. 2D) Chord diagrams of Vα-Jα and Vβ-Jβ combinations from the TCR clonotypes. The upper and lower parts of each semicircle represent V and J gene segments, respectively. Top 10 abundant V-J combinations are labeled. (FIG. 2E) Overlaps of the TCRα or TCRb CDR3 amino acid sequences. (FIG. 2F) Amino acid length distributions of TCRα or TCR CDR3s. P-values of CDR3 length distributions from two methods were calculated using Mann-Whitney U test. (FIG. 2G) Amino acid sequence motifs of TCRα or TCRB CDR3s identified by GLAM2.

FIGS. 3A-D. Single-cell RNA-seq analysis of flow cytometry-selected CD8⁺ T cells from Dextramer staining and ATLAS-seq. Five experiments were analyzed: input, Dextramer staining selected (2 replicates) and ATLAS-seq selected (2 replicates). (FIG. 3A) UMAP maps showing identified cell clusters. The major cell clusters (C₁-C₅) are colored. Pie charts: the proportions (%) of different cell clusters. (FIG. 3B) TCR clonotypes status (presence/absence) from scTCR-seq are projected onto the corresponding scRNA-seq UMAP maps. Cells with TCR clonotype information are colored blue. Pie charts: the proportions (%) of cells with TCR clonotype information. (FIG. 3C) Normalized average expression level of T cell marker genes for the 5 major cell clusters in Panel A. (FIG. 3D) Volcano plot shows the differential expressions of the immune regulation pathways in the major CD8⁺ T cell clusters in FIG. 3A. Green: pathways with activities significantly higher in ATLAS-seq hits than in Dextramer staining hits (fold change >10 and adjusted p-value <0.01). Red: pathways with activities significantly lower in ATLAS-seq hits than in Dextramer staining hits (fold change <−10 and adjusted p-value <0.01). The table: immune regulation pathways with highly differential activities in TCRs selected using the two methods.

FIGS. 4A-B. Feature comparisons of high-priority CMV-specific TCR clonotypes from Dextramer staining or ATLAS-seq. High-priority CMV-specific TCR clonotypes: with at least a total of 4 cells in the two replicates of each method. (FIG. 4A) Proportions of high-priority CMV-specific TCR clonotypes of each method. CMV-specific TCR clonotypes with at least 2 cells in at least one replicate were used as background. (FIG. 4B) Clonotype proportions, cytotoxic cytokine index and TCR-pMHC binding score comparison of high-priority CMV-specific TCR clonotypes of each method indicated in FIG. 4A. P-values were calculated by t-test.

FIGS. 5A-D. T Cell cytotoxicity comparisons of select CMV-specific clonotypes from Dextramer staining and ATLAS-seq. (FIG. 5A) Target cell killing assays: Activated TCR-transduced PBMCs were cocultured for 72 hours with target CMV⁺GFP⁺PC3 cells (GFP⁺PC3 cells expressing HLA-A2 and CMV pp65) or unrelated neuroblastoma SKNAS cells (negative for CMV pp65 peptide). Relative viability of CMV⁺PC3 cells is measured by GFP fluorescence using Incucyte system. Each dot: the average of three independent replicates. (FIG. 5B) Images of GFP expressing CMV⁺PC3 cells after the 72-hour coculturing with TCR A2, A3, A14, DL and D10 transduced PBMCs or un-transduced (UT) PBMCs. CMV⁺GFP⁺PC3 target cells are shown in green. Scale bar: 400 μm. (FIG. 5C) Comparison of target killing efficiencies (72-hour time point) of select CMV-specific clonotypes from Dextramer staining and ATLAS-seq. P-value is calculated by Mann-Whitney U test. **: p≤0.01, ***: p≤0.001 (FIG. 5D) Secreted IFNγ and TNFα concentrations after the 72-hour cell killing assay. Conditioned media in the same wells were measured by IFNγ and TNFα ELISA. Error bars: SD from three independent replicates.

FIGS. 6A-B. T2 cells can be used to present antigen peptides for T cell activation. (FIG. 6A) Images of IFNγ-specific aptamer beacon decorated control Jurkat cells or DMF5 Jurkat cells. These cells were co-cultured for 12 hours with T2 cells loaded without or with MART1 peptides. (FIG. 6B) Fluorescence intensity of each culture. Error bars: SD from three independent biological samples. P-values were calculated by t-test and *: p≤0.05, ns: p>0.05.

FIGS. 7A-D. ATLAS-seq microfluidic system. (FIG. 7A) Optimized single-T cell encapsulation performance in ATLAS-seq microfluidic chip. The droplets and their cell encapsulations were monitored by droplet imaging. Pie chart: Proportions (%) of droplets containing different number of cells. (FIG. 7B) Single T cells were encapsulated with culture media in droplets and incubated in 5 ml round-bottom tubes with different droplet layer thicknesses at 37° C., 5% CO2 for 2 days. T cell viabilities were measured by trypan blue exclusion test. Error bars: SD from three independent biological replicates. (FIG. 7C) The estimated proportions of droplets containing a given number of T2 cells or aAPC beads in encapsulation process using microfluidic device. The number of T2 cells per encapsulated volume follows Poisson distribution. The number of aAPC beads per encapsulated volume follows the Gaussian distribution. Different colors represent different input cell/bead concentrations in media. (FIG. 7D) Design of microfluidic chip for co-encapsulation of single T cells with aAPC beads in ATLAS-seq.

FIG. 8. Flow cytometry analysis of CMV antigen-specific CD8⁺ T cells identified by Dextramer staining and ATLAS-seq. T cells from human PBMCs were stimulated with aAPC beads loaded with the cognate peptide: CMV pp65 HLA-A2 (NLVPMVATV) and subject to either Dextramer staining or ATLAS-seq. Double positive cells in each method were selected for 10× Genomics single-cell sequencing. Input: the unstimulated CD8⁺ T cells from human PBMCs.

FIGS. 9A-B. Features of the high-priority CMV-specific TCR clonotypes of Dextramer staining or ATLAS-seq. Clonotypes are sorted by clonotype proportions (green bars, bottom axes) for Dextramer-staining (D1 to D80) or ATLAS-seq (A1 to A45) respectively. (FIG. 9A) Cytotoxic cytokine gene expression index. Blue: IFNγ; Orange: TNFα; Gray: LTA (TNFβ). (FIG. 9B) TCR-pMHC binding scores.

FIGS. 10A-E. Features of select CMV-specific TCRs from Dextramer staining and ATLAS-seq for target cell killing assay. (FIG. 10A) Dextramer staining or (FIG. 10B) ATLAS-seq clonotypes sorted according to clonotype proportions, cytotoxic cytokine indexes and TCR-pMHC-binding scores. Select CMV-specific TCRs for target cell killing assay were highlighted and labeled. (FIG. 10C) CMV antigen-specific CD8⁺ T cells identified by Dextramer staining using long-term (2 weeks) stimulation with aAPC beads loaded with the cognate peptide CMV pp65 HLA-A2 (NLVPMVATV). Flow cytometry analysis of CD8/PE double positive cells were selected for 10× Genomics single-cell TCR sequencing. Pie chart: the clonotype proportion of the dominate clonotype DL. (FIG. 10D) The correlations between target killing efficiencies and clonotype proportions, cytotoxic cytokine indexes or TCR-pMHC binding scores of select CMV-specific TCRs from Dextramer staining and ATLAS-seq. (FIG. 10E) Comparison of secreted IFNγ and TNFα concentrations after the 72-hour cell killing assay of select CMV-specific clonotypes from Dextramer staining and ATLAS-seq. P-value is calculated by Mann-Whitney U test. ***: p≤0.001, ****: p≤0.0001, ns: p>0.05.

### DESCRIPTION OF ILLUSTRATIVE EMBODIMENTS

As discussed above, there remains a need to more rapidly and accurately identify TCRs that have the potential to mediate antigen-reactive T cell activation in cancer and determine their TCR sequences. Such high-potential candidates for TCR therapy are in great need in oncology applications, and potentially other fields. To address these challenges, the inventors designed the Aptamer-based T Lymphocyte Activity Screening and sequencing (ATLAS-seq) technology, which isolates and characterizes activated T cells using an aptamer-based fluorescent molecular sensor to monitor cytokine secretion from T cells upon antigen stimulation, followed by single-cell sequencing. This microfluidic-based technology uses water-in-oil emulsions to provide a high-throughput approach to examine a single T cell's activation (as evidenced by cytokine release) after it interacts with artificial antigen-presenting cells (aAPCs) in droplets. Modified aptamers can be directly used on living cells without decreasing viability or needs of cellular gene manipulations. ATLAS-seq will efficiently and cost-effectively identify activated antigen-reactive T cells and provide a more fine-grained resolution to the identification of the most promising TCRs for immunotherapies.

These and other aspects of the disclosure are described in detail below.

### I. T Cell Receptors

The T-cell receptor (TCR) is a protein complex found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR.

The TCR is composed of two different protein chains (that is, it is a heterodimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes during ontogeny and in diseased states (such as leukemia). It also differs between species. Orthologues of the 4 loci have been mapped in various species. Each locus can produce a variety of polypeptides with constant and variable regions.

When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. Based on the initial receptor triggering mechanism, the TCR belongs to the family of non-catalytic tyrosine-phosphorylated receptors (NTRs).

The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

Each chain is composed of two extracellular domains: Variable (V) region and a Constant (C) region, both of Immunoglobulin superfamily (IgSF) domain forming antiparallel β-sheets. The Constant region is proximal to the cell membrane, followed by a transmembrane region and a short cytoplasmic tail, while the Variable region binds to the peptide/MHC complex.

The variable domain of both the TCR α-chain and β-chain each have three hypervariable or complementarity-determining regions (CDRs). There is also an additional area of hypervariability on the β-chain (HV4) that does not normally contact antigen and, therefore, is not considered a CDR.

The residues in these variable domains are located in two regions of the TCR, at the interface of the α- and β-chains and in the β-chain framework region that is thought to be in proximity to the CD3 signal-transduction complex. CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide. CDR2 is thought to recognize the MHC. CDR4 of the β-chain is not thought to participate in antigen recognition but has been shown to interact with superantigens. The constant domain of the TCR consists of short connecting sequences in which a cysteine residue forms disulfide bonds, which form a link between the two chains.

The TCR is a member of the immunoglobulin superfamily, a large group of proteins involved in binding, recognition, and adhesion; the family is named after antibodies (also called immunoglobulins). The TCR is similar to a half-antibody consisting of a single heavy and single light chain, except the heavy chain is without its crystallizable fraction (Fc). The two subunits of TCR are twisted together. Whereas the antibody uses its Fc region to bind to Fc Receptors on leukocytes, TCR is already docked onto the cell membrane. However, it is not able to mediate signal transduction itself due to its short cytoplasmic tail, so TCR still requires CD3 and zeta to carry out the signal transduction in its place, just as antibodies require binding to FcRs to initiate signal transduction. In this way the MHC-TCR-CD3 interaction for T cells is functionally similar to the antigen (Ag)-immunoglobulin (Ig)-FcR interaction for myeloid leukocytes, and Ag-Ig-CD79 interaction for B cells.

The generation of TCR diversity is similar to that for antibodies and B-cell antigen receptors. It arises mainly from genetic recombination of the DNA-encoded segments in individual somatic T cells by somatic V(D)J recombination using RAG1 and RAG2 recombinases. Unlike immunoglobulins, however, TCR genes do not undergo somatic hypermutation, and T cells do not express activation-induced cytidine deaminase (AID). The recombination process that creates diversity in BCR (antibodies) and TCR is unique to lymphocytes (T and B cells) during the early stages of their development in primary lymphoid organs (thymus for T cells, bone marrow for B cells).

Each recombined TCR possess unique antigen specificity, determined by the structure of the antigen-binding site formed by the α and β chains in case of αβ T cells or γ and δ chains on case of γδ T cells.

The TCR alpha chain is generated by VJ recombination, whereas the beta chain is generated by VDJ recombination (both involving a random joining of gene segments to generate the complete TCR chain).

Likewise, generation of the TCR gamma chain involves VJ recombination, whereas generation of the TCR delta chain occurs by VDJ recombination.

The intersection of these specific regions (V and J for the alpha or gamma chain; V, D, and J for the beta or delta chain) corresponds to the CDR3 region that is important for peptide/MHC recognition (see above).

It is the unique combination of the segments at this region, along with palindromic and random nucleotide additions (respectively termed “P-” and “N-”), which accounts for the even greater diversity of T-cell receptor specificity for processed antigenic peptides.

Later during development, individual CDR loops of TCR can be re-edited in the periphery outside thymus by reactivation of recombinases using a process termed TCR revision (editing) and change its antigenic specificity.

In the plasma membrane the TCR receptor chains α and β associate with six additional adaptor proteins to form an octameric complex. The complex contains both α and β chains, forming the ligand-binding site, and the signaling modules CD3δ, CD3γ, CD3ε and CD3ζ in the stoichiometry TCR αβ-CD3εγ-CD3ϵδ-CD3ζζ. Charged residues in the transmembrane domain of each subunit form polar interactions allowing a correct and stable assembly of the complex. The cytoplasmic tail of the TCR is extremely short, hence the CD3 adaptor proteins contain the signaling motifs needed for propagating the signal from the triggered TCR into the cell. The signaling motifs involved in TCR signaling are tyrosine residues in the cytoplasmic tail of these adaptor proteins that can be phosphorylated in the event of TCR-pMHC binding. The tyrosine residues reside in a specific amino acid sequence of the signature Yxx(L/I)x6-8Yxx(L/I), where Y, L, I indicate tyrosine, leucine and isoleucine residues, x denotes any amino acids, the subscript 6-8 indicates a sequence of 6 to 8 amino acids in length. This motif is very common in activator receptors of the non-catalytic tyrosine-phosphorylated receptor (NTR) family and is referred to as immunoreceptor tyrosine-based activation motif (ITAM). CD3δ, CD3γ and CD3ε each contain a single ITAM, while CD3ζ contains three ITAMs. In total the TCR complex contains 10 ITAMs. Phosphorylated ITAMs act as binding site for SH2-domains of additionally recruited proteins.

Each T cell expresses clonal TCRs which recognize a specific peptide loaded on an MHC molecule (pMHC), either on MHC class II on the surface of antigen-presenting cells or MHC class I on any other cell type. A unique feature of T cells is their ability to discriminate between peptides derived from healthy, endogenous cells and peptides from foreign or abnormal (e.g., infected or cancerous) cells in the body. Antigen-presenting cells do not discriminate between self and foreign peptides and typically express a large number of self-derived pMHCs on their cell surface and only a few copies of any foreign pMHC. For example, cells infected with HIV have only 8-46 HIV-specific pMHCs, compared with 100000 total pMHCs, per cell.^{[15][16]}

Because T cells undergo positive selection in the thymus, there is a non-negligible affinity between self-pMHC and the TCR. Nevertheless, the T-cell receptor signaling should not be activated by self-pMHC so that endogenous, healthy cells are ignored by T cells. However, when these very same cells contain even minute quantities of pathogen-derived pMHC, T cells must get activated and initiate immune responses. The ability of T cells to ignore healthy cells but respond when these same cells express a small number of foreign pMHCs is known as antigen discrimination.

To do so, T cells have a very high degree of antigen specificity, despite the fact that the affinity to the peptide/MHC ligand is rather low in comparison to other receptor types. The affinity, given as the dissociation constant (K_{d}), between a TCR and a pMHC was determined by surface plasmon resonance (SPR) to be in the range of 1-100 μM, with an association rate (kₒₙ) of 1000-10000 M⁻¹ s⁻¹ and a dissociation rate (k_{off}) of 0.01-0.1 s⁻¹. In comparison, cytokines have an affinity of KD=10-600 pM to their receptor. It has been shown that even a single amino acid change in the presented peptide that affects the affinity of the pMHC to the TCR reduces the T cell response and cannot be compensated by a higher pMHC concentration. A negative correlation between the dissociation rate of the pMHC-TCR complex and the strength of the T cell response has been observed. That means, pMHC that bind the TCR for a longer time initiate a stronger activation of the T cell. Furthermore, T cells are highly sensitive; interaction with a single pMHC is enough to trigger activation. T cells move on quickly from antigens that do not trigger responses, rapidly scanning pMHC on an antigen-presenting cell (APC) to increase the chance of finding a specific pMHC. On average, a T cell encounters 20 APCs per hour.

Different models for the molecular mechanisms that underlie this highly specific and highly sensitive process of antigen discrimination have been proposed. The occupational model simply suggests that the TCR response is proportional to the number of pMHC bound to the receptor. Given this model, a shorter lifetime of a peptide can be compensated by higher concentration such that the maximum response of the T cell stays the same. However, this cannot be seen in experiments and the model has been widely rejected. The most accepted view is that the TCR engages in kinetic proofreading. The kinetic proofreading model proposes that a signal is not directly produced upon binding, but a series of intermediate steps ensure a time delay between binding and signal output. Such intermediate “proofreading” steps can be multiple rounds of tyrosine phosphorylation. These steps require energy and therefore do not happen spontaneously, only when the receptor is bound to its ligand. This way only ligands with high affinity that bind the TCR for a long enough time can initiate a signal. All intermediate steps are reversible, such that upon ligand dissociation the receptor reverts to its original unphosphorylated state before a new ligand binds. This model predicts that maximum response of T cells decreases for pMHC with shorter lifetime. Experiments have confirmed this model. However, the basic kinetic proofreading model has a trade-off between sensitivity and specificity. Increasing the number of proofreading steps increases the specificity but lowers the sensitivity of the receptor. The model is therefore not sufficient to explain the high sensitivity and specificity of TCRs that have been observed. Multiple models that extend the kinetic proofreading model have been proposed, but evidence for the models is still controversial.

The antigen sensitivity is higher in antigen-experienced T cells than in naive T cells. Naive T cells pass through the process of functional avidity maturation with no change in affinity. It is based on the fact that effector and memory (antigen-experienced) T cell are less dependent on costimulatory signals and higher antigen concentration than naive T cell. Error! Bookmark not defined.

### II. Single-Cell Sequencing Methods

To profile a paired TCRαβ repertoire of T cells, several single-cell sequencing technologies have been developed to identify TCR sequences, analyze their antigen specificities, and pair TCRs with transcriptional and epigenetic cell state phenotypes in single cells. The following methods are exemplary and non-limiting.

Method 1: Single T cells are sorted into 96-well PCR plates. The first RT-PCR reaction is done using 76 TCR primers. In the second PCR, TCR transcripts from RT-PCR products are enriched by a multiplex PCR performed after reverse transcription (RT) reaction using a pool of forward primers spanning all the annotated productive Vα and Vβ fragments and reverse primers designed on the constant region of α and β chains. A third reaction is then performed that barcoded adaptors for each well are added by PCR enabling pooling. Then products from different wells are combined, purified, and sequenced. The resulting paired-end sequencing reads are assembled, deconvoluted and analyzed using a program called VDJFasta for TCR sequences.

Method 2: T cells are captured in water-in-oil droplets using microfluidic emulsion-based devices along with specific RT and PCR reagents. In each droplet, TCR α and β transcripts of a single cell are specifically reverse transcribed with RT primers designed on the constant region of α and β chain. cDNA is successively amplified using a pool of forward primers designed on all the alpha and beta segments and reverse primers designed on the constant region. α and β primers contain overlapping sequences at their 5′ ends that enable the synthesis of TCR alpha and beta fusion sequences through an overlap-extension mechanism. Fused molecules are pooled breaking the emulsion and further enriched by a nested amplification and sequenced. TCR reconstruction from single cell RNA sequencing (scRNA-seq) data.

Method 3: Single cells sorted in plates or captured using microfluidic devices are lysed, and total mRNA is reverse transcribed with an oligo dT primed reaction. Through a template-switch mechanism a universal sequence is added at the 5′ end of the transcript. This sequence shared with the dT primer used in the RT reaction is then used to amplify cDNA before library preparation. In the library preparation step, full-length cDNA is “tagmented” using transposase and Tag sequences inserted by transposase are then used to amplify cDNA and to insert barcoded sequencing adaptors. Libraries are then sequenced and TCR sequences can be extracted from all transcriptome using dedicated bioinformatics algorithms, such as TraCer, TraPes and VDJ Puzzle.

Method 4: Thousands of cells in parallel are partitioned into oil-in-water droplets. After a lysis step their mRNA is reverse transcribed using a pool of specific RT primers containing the same “cell barcode” used to tag the cell transcriptome, a Unique Molecular Identifier (UMI). UMI is different for each primer enabling the digital counting of mRNA transcripts and sequencing of the T7 promoter. cDNA is then amplified by in vitro transcription. After amplification barcoded RNAs are pooled and processed together. Amplified RNA is then used as template to enrich for TCR sequences and to generate RNA-seq libraries according to the inDrop protocol. During RNA-seq library preparation RNA is fragmented and only the 3′ end of transcripts is sequenced. For TCR enrichment amplified RNA is reverse transcribed using a pool of RT primers spanning V a and V β segments and then amplified using “internal” V a and β primers and primers designed on the constant regions. During this PCR reaction sequencing adaptors are also added. Libraries are then sequenced by Next-Generation Sequencing.

Method 5: Thousands of cells in parallel are partitioned into oil-in-water droplets. After a lysis step their mRNA is reverse transcribed using an oligo dT primer. Through a template-switch mechanism, a primer containing a cell barcode and a UMI is added at the 5′ end of the transcript. After RT reaction droplets are broken and cDNAs pooled and amplified using external primer designed on dT and switch oligonucleotides, respectively. Amplified full-length cDNA is then used as template to enrich TCR sequencing and and/or fragmented and processed to generate RNA-seq libraries. TCR enrichment is performed using nested PCR with a forward primer spanning the switch oligo and reverse primers designed on the constant region of α and β chains. PCR products are then partially fragmented and sequencing adaptors are added. Libraries are then sequenced by Next-Generation Sequencing.

### III. Screening Components

The presently described methods employ a number of specifically engineered reagents, including aptamer beacons that, when bound selectively by molecules produced by activated T cells, are detectable. The corresponding cytokines also play an important role in the process, as do antigen presenting cells, particularly those loaded with antigens to prime for T cell activation.

### A. Cytokine-Specific Aptamer Beacons

Aptamer beacon structure. Aptamers are single-stranded DNA (ssDNA), RNA oligonucleotide sequences which bind to their targets with high specificity and affinity. Because aptamers are short nucleic acid molecules, they are more robust than antibodies so that aptamer-based biosensors can be regenerated and used multiple times. Several strategies of transforming aptamer-analyte interactions into electrochemical, mechanical, piezoelectric, or fluorescent signals have been reported. Among these methods, fluorescence-based signal transduction is quite powerful because such strategies are highly sensitive and ensure rapid response and simple operation.

Fluorescence resonance energy transfer (FRET)-based aptamer or aptamer beacon is one of the most common methods used in real-time in vivo detection of cytokines. There are three typical aptamer beacon structures. (1) aptamer beacon is a ssDNA with stem-loop structure, which is labeled with a fluorophore and a quencher at each end. In the presence of the target cytokine, the aptamer stem is reconfigured the original unbound to a bound conformation separating the previously close fluorophore and quencher, and these structural changes upon ligand binding induce an increase in fluorescent signal. (2) aptamer beacon forms a duplex where a fluorophore-labeled aptamer is hybridized with an antisense oligonucleotide sequence carrying a quencher. The aptamer beacon shows no fluorescence in duplex, the presence of target cytokine forces the complementary strand to separate from the aptamer, thereby recovering the fluorescence of fluorophore. (3) By conjugating graphene oxide (GO) to dye-labeled aptamers, the fluorescent is adsorbed on the surface of GO via π-π interaction between the flat planar GO sheets and the ring structures in the nucleobases, resulting in fluorescence quenching of the dye due to a highly effective energy transfer from the dye to GO. The presence of the target causes the release of the aptamer from the GO surface which restores fluorescence.

Aptamer beacon immobilization on cell surface. Aptamer beacon can be immobilized on the cell surface by either covalent or noncovalent methods. Covalent conjugation mainly involves the modification of amino acids and sugar moieties. Noncovalent methods include hydrophobic insertion and molecular recognition.

Surface proteins have a large number of lysine and cysteine residues, which have amino or thiol groups, thereby allowing covalent conjugation with aptamer oligonucleotide. In general, when the amino group is used for conjugation, the first step is to convert the amino group into an intermediate reactive or binding group using a bifunctional crosslinker, such as N-hydroxy succinimide esters of biotin (NHS-biotin), then streptavidin-aptamer conjugates can bind biotin for cell-surface labelling.

Covalent conjugation can also be realized through the reaction of aptamer oligonucleotides with sugar moieties of membrane glycoproteins through metabolic pathways, such as N-azidoacetylmannosamine-tetraacylated (ManNAz), which is converted into an azido sialic derivative for protein glycosylation in cell. After the cell surface displays azido sugars, DNA oligonucleotides can be conjugated onto the cell surface through Staudinger ligation (azide-phosphine conjugation) or click chemistry (azide-alkyne conjugation).

Aptamer beacons can also be displayed on the cell membrane by noncovalent methods, which can be achieved by hydrophobic insertion of lipophilic residues, including cholesterol, tocopherol, C18 chains, diacyl phospholipid, etc. When aptamers conjugated with lipophilic residues are mixed with cells, the lipophilic residues will be inserted into the lipid bilayer and the hydrophilic aptamer strands will be located at the hydrophilic interface outside the membrane.

Another noncovalent conjugation for installing aptamer is through ligand-receptor binding, include the recognition of cell receptors by antibodies or aptamers. Typically, one can link receptor-specific antibody or aptamer with cytokine-specific aptamer to form antibody-aptamer conjugate or aptamer-aptamer oligo, then use them to recognize and bind the cell receptor for cell-surface labelling.

### B. Artificial Antigen Presenting Cells

Antigen-presenting cells (APCs). APCs are cells that displays antigen bound by major histocompatibility complex (MHC) proteins on its surface; this process is known as antigen presentation. T cells may recognize these complexes using their T cell receptors (TCRs). APCs process antigens and present them to T-cells.

Antigen-presenting cells are vital for effective adaptive immune response, as the functioning of both cytotoxic and helper T cells is dependent on APCs. Antigen presentation allows for specificity of adaptive immunity and can contribute to immune responses against both intracellular and extracellular pathogens. It is also involved in defense against tumors. Some cancer therapies involve the creation of artificial APCs (aAPCs) to prime the adaptive immune system to target malignant cells.

Antigen-presenting cells fall into two categories: professional and non-professional. Those that express MHC class II molecules along with co-stimulatory molecules and pattern recognition receptors are often called professional antigen-presenting cells. The non-professional APCs express MHC class I molecules.

T cells must be activated before they can divide and perform their function. This is achieved by interacting with a professional APC which presents an antigen recognized by their T cell receptor. The APC involved in activating T cells is usually a dendritic cell. T cells cannot recognize (and therefore cannot respond to) “free” or soluble antigens. They can only recognize and respond to antigen that has been processed and presented by cells via carrier molecules like MHC molecules. Helper T cells can recognize exogenous antigen presented on MHC class II; cytotoxic T cells can recognize endogenous antigen presented on MHC class I. Most cells in the body can present antigen to CD8+ cytotoxic T cells via MHC class I; however, the term “antigen-presenting cell” is often used specifically to describe professional APCs. Such cells express MHC class I and MHC class II molecules and can stimulate CD4+ helper T cells as well as cytotoxic T cells.

Artificial APCs. Artificial APCs (aAPCs) are the synthetic version of natural APCs and can be used in cancer immunotherapy for T cell expansion and activation. aAPCs are made by attaching the specific T-cell stimulating signals to various macro and micro biocompatible surfaces, which provide three key signaling components: (1) MHC I/TCR stimulatory signal. TCR agonists, such as recombinant peptide-MHC complexes or antibody directed to CD3, lead to the TCR ligation, which triggers the T cell activation. (2) CD80/CD28 costimulatory signal, which re upregulated on APCs and leads to complete activation of T cells, (3) cytokines produced by either APCs or T cells, which are essential for T cell expansion and differentiation. IL-2 is one of the most known cytokines for CD8⁺ T cell survival. Other cytokines such as IL-7, IL-15 and IL-21 have been investigated and may promote better expansion or differentiation into more optimal T cell phenotypes.

Genetically modified xenogeneic cells, such as Drosophila cells, murine fibroblasts, and human erythroleukemia cells have been used as aAPCs. They are easier to handle and better defined than natural APCs, allowing for more control over the signal delivered. These aAPCs are transfected to express specific peptide-loaded HLA molecules with co-stimulatory signal and cell adhesion molecules. K562-aAPC cell line is one of the most common approach, which is transfected with non-retroviral plasmids that encode for HLA-A*02:01 (A2), CD80 and CD83. The adhesion molecules ICAM-1 and LFA-3 expressed on K562 provides a effective T cell-APC interactions.

The other type of aAPCs is cell-sized, rigid, bead-based aAPCs, which provide a more simplistic systems for T cell activation because of their homogenous size distribution and straightforward functionalization. They are synthesized from various polymers, such as biodegradable poly lactic-co-glycolic acid (PLGA) and non-biodegradable sepharose or polystyrene. Immunomodulatory compounds, such as anti-CD3, anti-CD28 monoclonal antibodies and pMHC complexes can be anchored on the polymetric particle surface, while IL-2 or other soluble molecules can be gradually released from within aAPCs. The optimal size of sepharose or polystyrene beads is between 4 to 5 microns, of PLGA beads is between 6-10 microns which provides most effective activation of T cells.

### IV. Microfluidic Chip Materials and Droplet Generation Methods

Materials for microfluidic devices. Different materials lead to the design of microfluidic systems with a wide range of unique functionalities. These materials can be generally categorized as inorganic, organic, and hybrid or composite. In inorganic materials, silicon and glass are used to provide excellent optical transparency, well-defined surface chemistry and superior high-pressure resistance for microfluidics applications. In organic materials, polystyrene (PS), polyvinyl chloride, polymethyl methacrylate (PMMA), cyclic olefin co-polymers, polycarbonate, polytetrafluoroethylene (PTFE), polydimethylsiloxane (PDMS) and Flexdym are used to fabricate devices with facile surface modification, low cost, low thermal conductivity and compatibility with biomedical applications. Especially microfluidic devices made of PDMS or Flexdym are permeable to gases and are thus able to support long-term cell culture.

Droplet Generation Methods. Fine control over the size, shape, and mono disparity of droplets is of the key function of droplet microfluidics. In microfluidic channel, the emulsion is created by two immiscible fluids such as water and oil, and several methods are developed based on this basic principle. The following methods are exemplary and non-limiting.
Method 1: In the T-junction configuration, the inlet channel containing the intersection of the dispersed phase channel merges the continuous phase channel with the right angle. At the junction section, the interface can be observed between the two phases and as fluid passes the dispersed phase moves in the main channel. What lengthens the dispersed phase into the main channel is the shear forces that are caused by the continuous phase and the subsequent pressure gradient. The neck of the dispersed phase thins and eventually breaks the stream into a droplet.
Method 2: In the flow-focusing configuration, a narrow region forces the dispersed and continuous in the microfluidic device. Due to the symmetrical shear exerted by the continuous phase on the dispersed phase, the generated droplets are stable. The continuous break-off of droplets from the fluid at maximum shear point confirms the formation of uniform droplets.
Method 3: Di-electrophoresis (DEP) can be used to generate uniform droplets by pulling the droplets from a fluid reservoir. The force exerted on the uncharged fluid for droplet generation is caused by a non-uniform electric field.
Method 4: In a Electrowetting on dielectric (EWOD) device with two plane layers, the ground electrode is often placed on the top layer with the control electrodes on the bottom. Both layers include an insulating layer separating the droplets from the electrodes. Activation of the electrodes initiates fluid wetting of the channel and within tens of microseconds, the fluid begins to form a short liquid finger between the electrodes. The electrodes are then switched off, reverting the surface back to being hydrophobic. This causes the finger to break off from the reservoir and form a droplet.

### V. Methodologies

The disclosed methods employ well-known methodologies including droplet-based microfluidics and Fluorescence Activated Cell Sorting, which are discussed briefly below.

### A. Microfluidics

Microfluidics refers to the behavior, precise control, and manipulation of fluids that are geometrically constrained to a small scale (typically sub-millimeter) at which surface forces dominate volumetric forces. It is a multidisciplinary field that involves engineering, physics, chemistry, biochemistry, nanotechnology, and biotechnology. It has practical applications in the design of systems that process low volumes of fluids to achieve multiplexing, automation, and high-throughput screening. Typically, micro means one of the following features: small volumes (μL, nL, pL, fL), small size, low energy consumption and/or microdomain effects.

Typically, microfluidic systems transport, mix, separate, or otherwise process fluids. Various applications rely on passive fluid control using capillary forces, in the form of capillary flow modifying elements, akin to flow resistors and flow accelerators. In some applications, external actuation means are additionally used for a directed transport of the media. Examples are rotary drives applying centrifugal forces for the fluid transport on the passive chips. Active microfluidics refers to the defined manipulation of the working fluid by active (micro) components such as micropumps or microvalves. Micropumps supply fluids in a continuous manner or are used for dosing. Microvalves determine the flow direction or the mode of movement of pumped liquids. Often, processes normally carried out in a lab are miniaturized on a single chip, which enhances efficiency and mobility, and reduces sample and reagent volumes.

Droplet-based microfluidics is a subcategory of microfluidics in contrast with continuous microfluidics; droplet-based microfluidics manipulates discrete volumes of fluids in immiscible phases with low Reynolds number and laminar flow regimes. Interest in droplet-based microfluidics systems has been growing substantially in past decades. Microdroplets allow for handling miniature volumes (μl to fl) of fluids conveniently, provide better mixing, encapsulation, sorting, and sensing, and suit high throughput experiments. Exploiting the benefits of droplet-based microfluidics efficiently requires a deep understanding of droplet generation to perform various logical operations such as droplet manipulation, droplet sorting, droplet merging, and droplet breakup.

Chip-based microfluidics have greatly enhanced applications in many different fields. Early biochips were based on the idea of a DNA microarray, e.g., the GeneChip DNAarray from Affymetrix, which is a piece of glass, plastic or silicon substrate, on which pieces of DNA (probes) are affixed in a microscopic array. Similar to a DNA microarray, a protein array is a miniature array where a multitude of different capture agents, most frequently monoclonal antibodies, are deposited on a chip surface; they are used to determine the presence and/or amount of proteins in biological samples, e.g., blood. A drawback of DNA and protein arrays is that they are neither reconfigurable nor scalable after manufacture. Digital microfluidics has been described as a means for carrying out Digital PCR.

In addition to microarrays, biochips have been designed for two-dimensional electrophoresis, transcriptome analysis, and PCR amplification. Other applications include various electrophoresis and liquid chromatography applications for proteins and DNA, cell separation, in particular, blood cell separation, protein analysis, cell manipulation and analysis including cell viability analysis and microorganism capturing.

### B. Fluorescence-Activated Cell Sorting

Fluorescence-Activated Cell Sorting (FACS), is also known as flow cytometry cell sorting, or commonly known by the acronym FACS, which is a trademark of Beckton Dickinson and Company. Fluorescence activated cell sorting utilizes flow cytometry to separate cells based on morphological parameters and the expression of multiple extracellular and intracellular proteins. This method allows multiparameter cell sorting and involves encapsulating cells into small liquid droplets which are selectively given electric charges and sorted by an external electric field. Fluorescence activated cell sorting has several systems that work together to achieve successful sorting of events of interest. These include fluidic, optical, and electrostatic systems. The fluidic system has to establish a precisely timed break off from the liquid stream in small uniform droplets, so that droplets containing individual cells can then be deflected electrostatically. Based on the invention of Richard Sweet, droplet formation of the liquid jet of a cell sorter is stabilized by vibrations of an ultrasonic transducer at the exit of the nozzle orifice. The disturbances grow exponentially and lead to break up of the jet in droplets with precise timing. A cell of interest that should be sorted is measured at the sensing zone and moves down the stream to the breakoff point. During the separation of the droplet with the cell in it from the intact liquid jet, a voltage pulse is given to the liquid jet so that droplets containing the cells of interest can be deflected in an electric field between two deflection plates for sorting. The droplets are then caught by collection tubes or vessels placed below the deflection plates.

Flow cytometry cell sorting yields very high specificity according to one or several surface markers, but one limitation is constituted by the number of cells that can be processed during a workday. For this reason, pre-enrichment of the population of interest by immunomagnetic cell sorting is often considered, especially when the target cells are comparatively rare and a large batch of cells must be processed. Moreover, flow cytometry cell sorters are complex instruments that are generally used only by well-trained staff in flow cytometry facilities or well-equipped laboratories and, since they are normally big in size, it is not always possible to place them inside a biological safety cabinet. Therefore, it is not always possible to ensure sample sterility and, since the fluidic systems can be cleaned but it is not single-use, there is the possibility of cross-contamination among samples. Another aspect to be considered is that droplet generation inside the instrument could lead to aerosol formation that are hazardous for the operator when using infectious samples. These last considerations are of particular importance when cell sorting is used for clinical applications, for example cell therapy and should therefore be performed under Good Manufacturing Practice (GMP) conditions. Researchers can use a variety of fluorescent dyes to design multi-color panels to achieve successful, simultaneous sorting of multiple, precisely defined cell-types.

Due to various limitations of fluorescence-activated and immunomagnetic cell sorting devices, a wide range of microfluidic cell-sorting devices have emerged. A few of these are now commercially available or in commercial development. Research into microfluidic cell sorter designs often employ soft lithography techniques utilizing materials such as polydimethylsiloxane (PDMS).

A key benefit of microfluidic sorters is the potential to perform fluorescence-activated cell sorting in a closed single-use sterile cartridge. Such a closed cartridge would prevent the exposure of an operator to biohazards through the droplets that are emitted by FACS systems. Other benefits include reduced impact on cell viability due to reduced hydrodynamic stress on the cells; Some published devices show the potential for multi-way sorting, decreased cost of a cartridge due to low-cost manufacturing methods, lower power consumption, and smaller-sized footprints, with some devices being the size of a credit card. Some have achieved high purity outputs and rates of up to around 50,000 cells/s.

Microfluidic cell sorters can be divided into two categories: active and passive. Active devices deflect individual cells by the cytometric measurements of the cells, made in real time. Passive devices exploit physical differences between cells in how they interact with the fluid flow or surfaces.

Active microfluidic cell sorters involve the deflection of individual cells following their measurement using cytometric methods, including fluorescent labelling, light scatter and image analysis. Individual cells are deflected by either a force directly on the cell or a force on the fluid surrounding the cells, so that they flow into separate output vessels.

Methods of cell deflection employ several kinds of macroscopic, optical or MEMS (micro-electro-mechanical systems) actuators to deflect a particle or liquid volume within a microchannel. Notable recent examples are based on surface acoustic wave actuators, macroscopic actuators (such as piezoelectric actuators) coupled to microchannels, dielectrophoresis of droplets, thermal vapor bubble actuators, transient micro-vortices generated by thermal vapor bubble actuators, optical manipulation, and micro-mechanical valves. The fastest of these have demonstrated sort rates in excess of 1000/s and potential maximum throughput rates in some cases approaching those of FACS. Active microfluidic cell sorting requires similar cytometry instrumentation as fluorescence-activated cell sorting described above.

Active microfluidic cells sorters have the potential of throughput scaling by parallelisation on chip. The fastest published active microfluidic sorting device has demonstrated a 160,000/s throughput.

Passive cell sorting uses the behavior of the fluid within the microchannels to alter and separate cells based on size and morphology. The fluid in a colloidal solution is subject to a velocity profile due to the interactions of the fluid with the walls of the channel; the cells in the solution are subject to various drag and inertial forces that are dependent on the size of the cell and balance accordingly at different locations along the velocity profile. In curved microfluidic channels, vortices are formed due to the Dean force which locate different sized particles in different cross-sectional locations due to the Reynolds number and curve radius of curvature.

For example, in a straight channel, larger cells in colloidal solution are found closer to the center of the microchannel than smaller cells due to the larger drag forces from the wall that pushes the cell away from the wall and the shear gradient force from the velocity profile that balances this wall drag force to set the cell into equilibrium.

### VI. Cancer Therapies

The materials identified by the disclosed methods will be useful in treating cancers. Types of cancers to be treated with the binding agents of the disclosure include, but are not limited to, hematological cancers, solid tumors, and non-solid tumors. Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases). Adult tumors/cancers and pediatric tumors/cancers are also included.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of binding agent, or a material such as an antibody-drug conjugate, composition or dosage form comprising an active binding agent, which is effective for producing some desired therapeutic effect when administered in accordance with a desired treatment regimen.

The subject/patient may be an animal or any species of mammal, including, without limitation, a horse, a dog, a cat, a pig, or a primate. In a particular embodiment, the subject/patient is a human.

In certain embodiments, the compositions identified by the presently disclosed methods may be used in combination with at least one additional therapy. The additional therapy may be radiation therapy, surgery (e.g., lumpectomy and a mastectomy), chemotherapy, gene therapy, DNA therapy, viral therapy, RNA therapy, another immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An additional therapy may be administered before, during, after, or in various combinations relative to the T Cell Receptor Therapy described herein. The administrations may be in intervals ranging from concurrently to minutes to days to weeks. In some embodiments where the therapies are provided to a patient separately, one would generally ensure that a significant period of time did not expire between the time of each delivery, such that the two treatments would still be able to exert an advantageously combined effect on the patient. In such instances, it is contemplated that one may provide a patient with the both therapies within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below a T cell receptor therapy is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any therapy of the present embodiments to a patient will follow general protocols for the administration of such compounds, taking into account the toxicity, if any, of the agents. Therefore, in some embodiments there is a step of monitoring toxicity that is attributable to combination therapy.

Chemotherapy. A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

Radiotherapy. Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Immunotherapy. The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons ÿ, ÿÿ and ÿ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO″, is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., (1998) Proc Natl Acad Sci USA 95 (17): 10067-10071; Camacho et al., (2004) J Clin Oncology 22 (145): Abstract No. 2505 (antibody CP-675206); and Mokyr et al., (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VII. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Microfluidic chip fabrication. The photomask of microfluidic chip of ATLAS-seq was printed by Fineline Imaging. The master for microfluidic chip fabrication was generated on a silicon wafer (WaferPro, C04007) by photolithography through ABM 3000 HR Mask Aligner (ABM) at Singh Center for Nanotechnology (PA, U.S.A). Polydimethylsiloxane (PDMS) prepolymer and its cross-linking agent (Fisher Scientific, NC9285739) were mixed in 10:1 mass ratio and vacuum degassed, then the mix was poured onto the silicon wafer master to make the PDMS slab at 80° C. for at least 30 min. After the mold was cut and peeled off from the wafer, the inlet/outlet ports were punched. The mold was bonded on a clean glass plate in Tergeo plasma cleaner (PIE Scientific, Tergeo) with 50W treatment for Imin, then placed on a hotplate at 60° C. for 1 h to improve the bond. Aquapel (Aquapel Glass Treatment, 47100) treatment was then performed to keep the inside of the channels hydrophobic.

aAPC beads preparation and peptide loading. To prepare aAPC beads, 1 ml M-450 Epoxy beads (Invitrogen, 14011) were washed with 1 ml 0.1M borate buffer, then resuspended in a mixture of 1 ml 0.1M borate buffer with 20 μg of HLA-A2-Ig dimer (BD Biosciences, 551263) and 20 μg of anti-human CD28 (Clone 9.3, Bio X Cell, BE0248R005MG). The bead mixture was rotated at 4° C. for 24 hours and washed twice with 1 ml aAPC wash buffer (950 ml DPBS, 30 ml human AB serum (MilliporeSigma, H4522-100ML), 2 mM EDTA, 0.1g sodium azide, filter through 0.22 μm filter and store at 4° C.). The beads were then incubated in 1 ml aAPC wash buffer and rotated at 4° C. for another 24 hours. The prepared aAPC Bead quality was assessed by staining with anti-mouse IgG1-PE (Invitrogen, P-21129) and anti-mouse IgG2a-FITC (BD Biosciences, 553390) in flow cytometry staining buffer (Invitrogen, 00-4222-26). To load antigen on aAPC beads, 10 μl of 1 mg/ml antigen peptides were mixed with PBS washed aAPC beads and incubated at 4° C. for 3 days. Antigen loaded aAPC remained functional for at least 6 months when stored in suspension at 4° C.

T cell isolation, culture and expansion. Pan T cells were isolated from human PBMCs (Cellular Technology Limited, LP_501) with human pan T cell isolation kit (Miltenyi, 130096535) and cultured in T cell culture medium (X-VIVO 15 media supplemented with 5% human serum and 300U/ml Interleukin-2 (IL-2)). To expand Pan T cells, in each well of a 24-well plate, 1×10⁶ pan T cells were resuspend in 1 ml culture medium with 1×10⁶ CD3/CD28 beads (Gibco, 11131D) and incubate at 37° C., 5% CO2 for 14 days. After expansion, pan T cells were resting in T cell culture medium without CD3/CD28 beads for 2 days before proceeding downstream experiments. From these rested pan T cells, CD8⁺ T cells were isolated with human CD8⁺ T cell isolation kit (Miltenyi, 130096495). Isolated CD8⁺ T cells can be frozen in 5% DMSO in FBS for cryopreservation in liquid nitrogen.

Aptamer decoration of T cells. IFNγ-specific aptamer beacon (/5Cy3/AGGGGTTGGTTGTGTTGGGTGTTGTGTCCAACCCCT/TAO//iSp18//3CholTEG/(SEQ ID NO: 115)) was synthesized by IDT. Three hours before the ATLAS-seq procedure, 1×10⁶ CD8⁺ T cells were resuspended in 50 μl DPBS, transferred to one well of a laminar wash plate (Curiox, U.S.A) and washed 5 times in Laminar Wash HT2000 (Curiox). Then washed 25 μl CD8⁺ T cells were resuspended in the laminar wash plate with 10 μl 100 μM IFN-γ aptamer beacon and 15 μl DPBS, followed by a 30-minute incubation at room temperature in dark. The aptamer-decorated T cells were then washed 5 times in Laminar Wash HT2000 and resuspended in T cell culture medium (no IL-2) with 20% OptiPrep™ Density Gradient Medium (MilliporeSigma, D1556) to 6×10⁵ cell/ml for ATLAS-seq.

Co-culture of aptamer decorated T cells and aAPC in droplets. Aptamer decorated CD8⁺ T cells are prepared in 6×10⁵ cell/ml suspension in T cell culture medium (no IL-2) with 20% OptiPrep™ Density Gradient Medium and loaded in 1 ml sterile syringe. aAPC beads are prepared in 5×10⁷ bead/ml suspension in T cell culture medium with 600U/ml IL-2 and 20% OptiPrep™ Density Gradient Medium and loaded in 1 ml sterile syringe. HFE7500 oil (3M, Novec, 7500) with 2% (w/w) surfactant (Ran Biotech, 008-FluoroSurfactant-2wtH-50G) was loaded in Another sterile syringe. All three syringes were connected to the inlet ports of the co-encapsulation microfluidic chip through tubing and were controlled by inDrop pump system (1CellBio, U.S.A). The following flow rates were used: 600 μl/h oil, 200 μl/h T cell suspension and 200 μl/h aAPC suspension. The droplets were collected in a 5 ml round-bottom tube for 20 min per tube, followed by a 2-day incubation at 37° C. in 5% CO2. After removing the oil, 500 μl fresh culture medium and 500 μl 20% (vol/vol) 1H,1H,2H,2H-Perfluoro-1-octanol (MilliporeSigma, 370533) in HFE7500 oil were added on top of the emulsion to break the droplets by incubation at room temperature for 2 min. The liquid phase on top was transferred into a new tube and aAPC beads were removed from cell suspension using DynaMag™-2 magnet rack (ThermoFisher, 12321D). Recovered CD8⁺ T cells were prepared for fluorescence-activated cell sorting (FACS) using the following steps: 1) Resuspend the T cells in 50 μl DPBS and wash 5 times in Laminar Wash HT2000. 2) Add 25 μl staining buffer (2% FBS in DPBS) and 5 μl anti-CD8 antibody (3B5), Pacific Blue™ (ThermoFisher, MHCD0828), mix and incubate in dark at room temperature for 30 min. 3) Wash cells 5 times in Laminar Wash HT2000. 4) Resuspend the T cells in 500 μl DPBS with 10% FBS for FACS sorting.

Dextramer Staining. After a two-day co-incubation of CD8⁺ T cells and aAPC beads in 1:1 ratio in T cell culture medium at 37° C. in 5% CO2, CD8⁺ T cells were separated from aAPC beads using DynaMag™-2 magnet rack and washed twice by PBS. 1×10⁶ washed CD8⁺T cells were resuspended in 50 μl Dextramer stain buffer (PBS containing 1% human serum and 0.1 g/l Herring sperm DNA (ThermoFisher, 15634017)). Add 2.2 μl antigen-specific Dextramer reagent (0.2 μl 100 μM d-Biotin and 2 μl dCODE™ Dextramer® (Immundex)) to the CD8⁺ T cell suspension and mix thoroughly. Transfer the mix into a well of a laminar wash plate and incubate in dark at room temperature for 10 min, then add 5 μl anti-CD8 antibody (3B5), Pacific Blue™ into the same well of the plate and incubate in dark at room temperature for another 20 min. CD8⁺ T cells were then washed 5 times in Laminar Wash HT2000 and resuspended in 500 μl DPBS with 10% FBS for FACS sorting.

Single-cell TCR and RNA sequencing library preparation and data analysis. After cell sorting, collected T cell suspension was transferred to laminar wash plate (˜80 μl in each well) and incubated at room temperature for 30 min, followed by washing 5 times with DPBS in Laminar Wash HT2000. The protocol of 10× Genomics Chromium Next GEM Single Cell 5′ Kits were used to resuspend T cells into a proper concentration to achieve targeted cell recovery and prepare the V (D) J library for single-cell TCR sequencing. Sequencing data was processed by using the pipeline of 10× Genomics Cell ranger (PMID: 28091601) and Seurat (PMID: 34062119, PMID: 34062119). The differentiated immune regulation pathway activities were analyzed by SCPA package (PMID: 36417885). The TCR clonotypes were analyzed by VDJtools (PMID: 26606115). Sequencing data are available at Gene Expression Omnibus. The TCR-pMHC binding scores of TCR clonotypes for CMV pp65 peptide (NLVPMVATV) were calculated by NetTCR-2.0 based on both TCRα and TCRβ CDR3s (PMID: 34508155).

TCR cloning and retroviral packaging. Full-length DNA fragments of TCRα and TCRβ were synthesized using gBlocks™ gene fragments (IDT) with 30 bp overlapping sequences (Table.S2). TCR expression vectors were constructed by mixing TCRα and TCRβ g-Blocks with a linearized pMSCV-IRES-mCherry FP (pMIR) expression vector (a gift from Vignali plasmid Dario (Addgene #52114; http://n2t.net/addgene: 52114; RRID: Addgene_52114)), followed by a three-way ligation using Gibson Assembly Cloning (NEB, E2611S) at 50° C. for 15 min and store on ice. The resulting pTCR-MIR expression vectors encoded TCRα and TCRβ fragments connected by a 2A self-cleaving peptide P2A. During translation, TCRα and TCRβ fragments are separated through P2A induced ribosomal skipping and form fully functional TCR of interest-Following the protocol of Lipofectamine 3000 (Invitrogen, L3000015), Phoenix retroviral producer cells (ATCC, CRL-3214) were transfected with equal amount of pTCR-MIR and pCMV-VSV-G vectors (Cell Biolabs, RV-110) at 2.5 μg/1×10⁶ cells concentration and incubated at 37° C., 5% CO₂ for 2 days. Then, from transfected Phoenix retroviral producer cell medium, retroviral particles were precipitated with PEG-it virus precipitation solution (SBI, LV810A-1) at 4° C. overnight followed by spinning at 1,500 xg for 30 min and resuspended in serum-free X-vivo15 medium. Retrovirus-coated plates for PBMC transduction were prepared as follows: RetroNectin (Takara, T100A) coated 6-well plates containing 2 ml/well retroviral particle suspension were centrifuged at 2,000×g for 2 h at 32° C. Viral supernatant was aspirated and replaced with 2 ml PBMC growth medium (X-vivo 15 media supplemented with 10% heat-inactivated FBS, 1x penicillin/streptomycin/L-glutamine and 50U/ml IL-2).

PBMC Transduction. 2×10⁶ cells/mL healthy PBMCs (Cellular Technology Limited, LP_159) were stimulated with CD3/CD28 beads in PBMC growth medium at 37° C., 5% CO₂ for 2 days. After removing CD3/CD28 beads, PBMCs were collected with centrifugation at 600×g for 5 min at 25° C. PBMCs were resuspend in PBMC growth medium to 1.5×10⁶ cells/mL and 1 ml cell suspensions were added to each viral-coated well at 1.5×10⁶ cells/3 mL/well. Plates were centrifuged at 1,000×g for 10 min at 32° C., then incubated at 37° C., 5% CO2 for 2 days. PBMCs were then collected and pelleted at 600×g for 5 min at 25° C. Transduced PBMCs were resuspended in PBMC growth media with 1% 100× β-mercaptoethanol solution (MilliporeSigma, ES-007-E) and incubated at 37° C., 5% CO2 for 4 days. Transduced PBMCs can be frozen in FBS with 5% DMSO for cryopreservation in liquid nitrogen.

Cytotoxicity assays. 0.2×10⁵/well target GFP⁺PC3 cells expressing HLA-A2 and CMV pp65 (a gift from Owen's lab) were seeded in 96-well plates at 37° C., 5% CO2 overnight. Then, 2×10⁵ TCR-transduced PBMCs mixed with 6×10⁵ CMV pp65 loaded aAPC beads for activation were added to each well for coculturing with target cells. Incucyte S3 (Sartorius, Germany) was used to monitor and measure GFP⁺PC3 cell viabilities via GFP fluorescence intensities for 72 h. Relative viabilities of target cells were calculated by non-treated target cell normalized GFP intensities. Target killing efficiencies of clonotypes were calculated as the relative viability decreasing after the 72h assay. Supernatants of each assay was collected for IFNγ and TNFα ELISA using IFN gamma Human ELISA Kit (Invitrogen, KHC4021) and TNF alpha Human ELISA Kit (Invitrogen, KAC1751).

### Example 2—Results

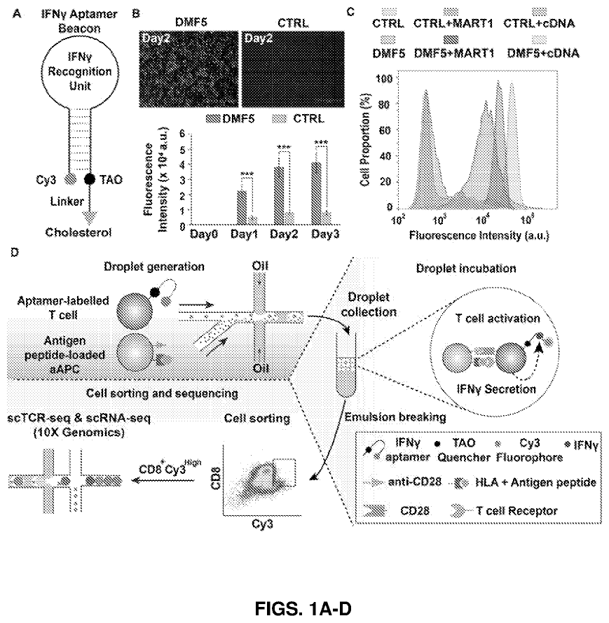

IFNγ-specific aptamer beacon can be used as a reporter for antigen-specific T cell activation. The inventors constructed a membrane anchored IFNγ aptamer beacon according to previous report (Qiu, Wimmers et al., 2017). Briefly, an aptamer IFNγ recognition unit is extended by a 3′-end 9mer DNA sequence complementary to the 5′-end of the aptamer, which can lead to the formation of a stem-loop structure due to self-hybridization. The aptamer is modified with a 5′-Cy3 fluorophore and a 3′-TAO quencher, which is further connected to a cholesterol modification by an iSP18 linker sequence (FIG. 1A). This aptamer beacon can anchor onto cell membrane through the hydrophobic interaction between cholesterol and phospholipid (Qiu, Wimmers et al., 2017). In the absence of IFNγ, the stem-loop structure brings Cy3 and TAO quencher in close proximity, which in turn leads to the quenching of the fluorescence. Upon binding with IFNγ, the aptamer beacon switches to a tertiary structure that separates Cy3 from the TAO quencher and allows the emission of fluorescence signal.

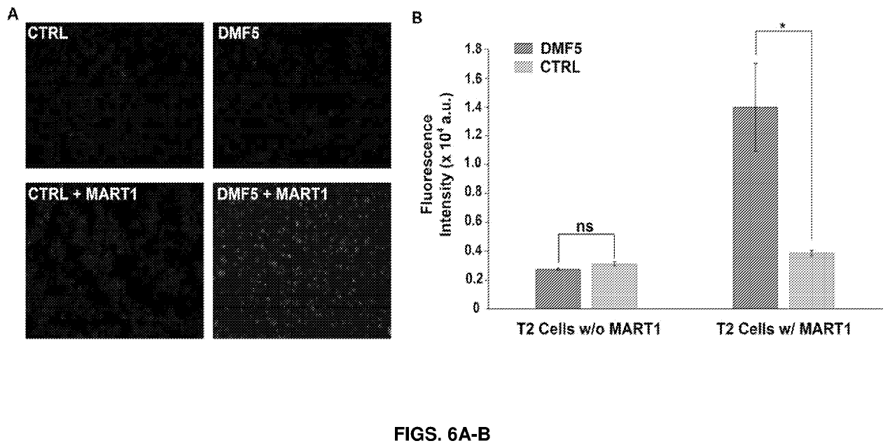

The inventors first tested the reporter function of IFNγ specific aptamer in antigen-specific T cell activation using artificial antigen-presenting cells (aAPCs). aAPCs were prepared by coupling superparamagnetic epoxy beads with human HLA-A2-Ig dimer and anti-CD28Ab (Chiu, Schneck et al., 2011; Neal, Bailey et al., 2017). Myeloma specific MART1 peptides were presented by aAPCs and can be specifically recognized by the TCR DMF5 (Borbulevych, Santhanagopolan et al., 2011). Jurkat cells with or without DMF5 expression were decorated with IFNγ specific aptamer beacon and co-incubated with MART1 peptide-loaded aAPCs. The fluorescence signal on DMF5 Jurkat cells increased significantly over time, compared to the unchanged low baseline fluorescence signals detected on control Jurkat cells (FIG. 1B). Additionally, when using antigen presenting T2 cells (Cole, Weil et al., 1995; Lyons, Moore et al., 2006) to present MART1 peptides to Jurkat cells with or without DMF5 expression, significantly higher fluorescence signal was also observed on DMF5 Jurkat cells than the baseline fluorescence signal detected in control Jurkat cells (FIGS. 6A-B). These results suggest that the IFNγ specific aptamer beacon can be used to efficiently detect IFNγ secretion from the antigen-specific T cells activated with either antigen presenting aAPC beads or T2 cells.

Design and optimization of microfluidic co-encapsulation of T cells and antigen presenting cells. The inventors hypothesize that IFNγ specific aptamer beacon can be used to distinguish the antigen-activated T cells from non-activated T cells when single T-cells are co-encapsulated with antigen presenting aAPCs or T2 cells in water-in-oil droplets. The co-encapsulation can create spatial isolation of IFNγ secreted from each T cell and prevent the crosstalk between different aptamer-loaded T cells upon activation.

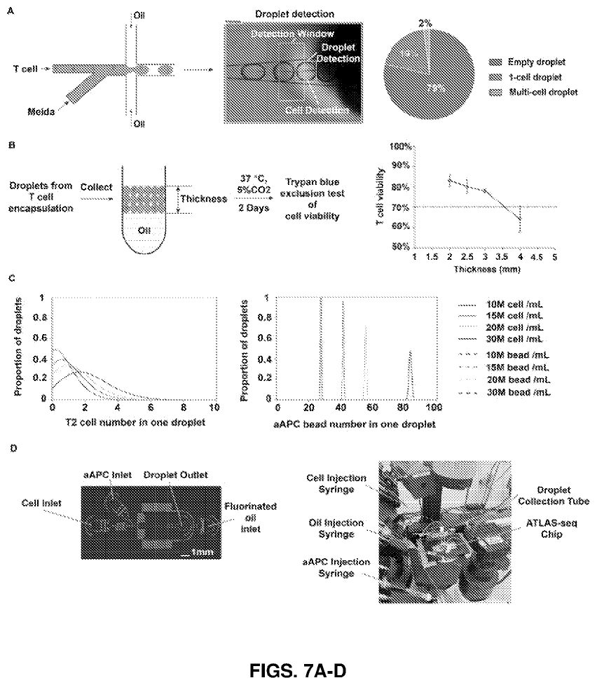

The inventors designed and optimized single-T cell encapsulation procedure based on COMSOL Multiphysics simulation. Droplets generated from the single-T cell encapsulation procedure were examined using a machine vision system based on a FLIR high-speed camera (FIG. 7A left and middle). With the optimized setting, among all generated droplets, 19% contained single T cell, 2% contained multiple T cells and 79% were empty (FIG. 7A right).

Based on the result in FIG. 1B, the inventors chose to use 2-day co-incubation for the activation of T cells with antigen presenting cells. To satisfy the requirement of single-cell sequencing and the efficiency of sample preparation, a minimum of 70% T cell viability is required. Encapsulated T cells show reduced viabilities due to oxygen deprivation when incubated in a thick layer of water-in-oil droplets. To maximize the viabilities of the encapsulated T cells after the 2-day incubation, the inventors compared the T cell viabilities after incubating in different thicknesses of the T cell containing droplet layer. The droplet layer thicknesses no more than 3 mm resulted in >75% T cell viability (FIG. 7B). As a result, 3 mm was chosen to be the maximum thickness for the collected droplet layer for incubation.

To compare the performances of T2 cells and aAPCs in the encapsulation process, the inventors simulated their droplet encapsulation statistics in cell culture media. Because the density of a cell is between 1 to 1.3 g/mL and similar to the cell culture media (about 1 g/mL), cells are distributed randomly in culture media suspension, and the number of cells per droplet follows Poisson statistics (Collins, Neild et al., 2015). According to the simulation, the number of encapsulated T2 cells in each droplet ranges widely between 0 to 8 cells per droplet, depending on the input T2 cell concentrations (FIG. 7C left). More importantly, large proportions of the droplets (10%-50%) will not contain any T2 cells (FIG. 7C left). In contrast, the density of an aAPC bead is about 2 g/mL and the diameter (4.5 μm) is much smaller than a T2 cell (12 μm). As a result, aAPC beads form a close-packing of equal spheres before entering the site of droplet formation, which allows the number of beads per droplet to be proportional to the Gaussian distributed droplet size (Peng, Mansson et al., 2019). According to the simulation, the number of encapsulated aAPC beads in each droplet is approximately equal and much higher than the number of T2 cells when using the same input concentration (FIG. 7C right). These results suggest aAPC beads are more efficient than T2 cells in droplet co-encapsulation process. Using magnetic aAPC beads will also make the separation of selected T cells in downstream application more efficient than using T2 cells.

To co-encapsulate single CD8⁺ T cell with aAPC beads, the inventors designed a microfluidic chip for droplet generation (FIG. 7D). In this chip, two inputs are simultaneously injected into the microfluidic chip with the same flow rate: 1) aptamer beacon decorated CD8⁺T cells in media suspension without IL-2; 2) antigen peptides loaded aAPC beads in media suspension with IL-2. The two aqueous streams are merged and cut into ˜120 μm diameter droplets by injected oil flow.

Aptamer-based T Lymphocyte Activity Screening and Sequencing (ATLAS-seq) allows high-throughput selection of highly activated antigen specific T-cells. Using the optimized set-ups described above, the inventors tested the utility of using IFNγ specific aptamer beacon to identify single T-cell activation in droplets. Jurkat cells with or without DMF5 expression were decorated with IFNγ specific aptamer beacon and co-encapsulated, in single-cell droplets, with aAPC beads loaded either with or without MART1 peptides. DNA oligo complementary to the IFNγ specific aptamer beacon stem region (5′-AGGGGTTGGACACAACACCCAACACAACCAACCCCT-3′ (SEQ ID NO: 116)), referred to as “cDNA” in FIG. 1C) were used to treat the aptamer beacon decorated Jurkat cells as a reference for the range of maximum fluorescence signal that can be emitted from the aptamer beacons on these cells. After two-day incubation, the Jurkat cells were released from the droplets and the fluorescence signal on the surface of each cell was measured by flow cytometry. The fluorescence signals on DMF5 Jurkat cell cocultured with MART1 aAPCs (FIG. 1C, dark blue) were significantly higher than on those cocultured with empty aAPCs (FIG. 1C, gray), and closer to the maximum fluorescence signal range indicated by the “cDNA” treatments (FIG. 1C, light blue). In contrast, the fluorescence signals on control Jurkat cells with or without MART1 aAPCs remain similar low baseline levels (FIG. 1C, orange and green).

Based on the above results, the inventors established a high-throughput Aptamer-based T Lymphocyte Activity Screening and Sequencing (ATLAS-seq) workflow to efficiently select highly reactive antigen-specific TCRs (FIG. 1D). Briefly, they used the microfluidic chip to generate droplets co-encapsulating aptamer decorated single CD8⁺ T cell and antigen-loaded aAPCs. Generated droplets were collected and incubated for 2 days to allow T-cell activation by antigen-loaded aAPCs. An activated T-cell containing cognate TCR for the target antigen pMHC may release IFNγ molecules into the same droplet which, in turn, bind to the IFNγ specific aptamer beacons on the same T-cell surface and emit Cy3 fluorescence signal. The droplet emulsions were then broken to release aptamer labelled CD8⁺ T cells for fluorescent sorting using flow cytometry. CD8⁺Cy3^{high} T cells were then collected and subject to single cell TCR and RNA sequencing (scTCR-seq and scRNA-seq) prepared by 10× Genomics single-cell sequencing system.

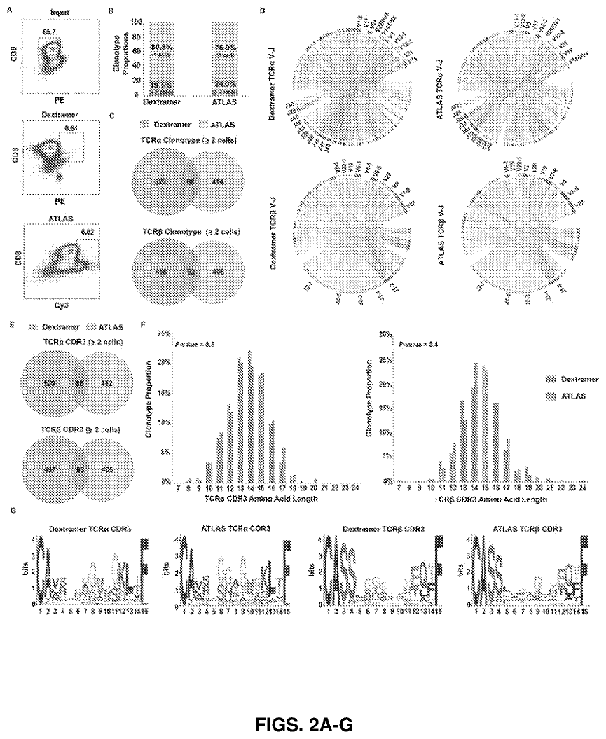

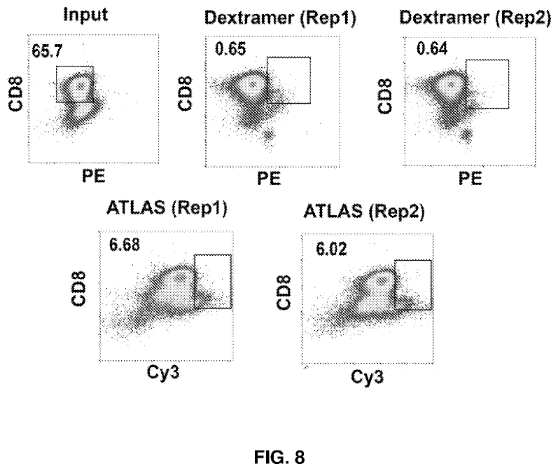

ATLAS-seq recovered a distinct antigen-specific TCR population from the one recovered by the Dextramer staining method. To examine the TCR clonotype population detected by ATLAS-seq, the inventors isolated CD8⁺ T cells from the PBMCs of a CMV positive donor and expanded them with CD3/CD28 beads. After resting, these expanded CD8⁺T cells were used as the input for either Dextramer staining or ATLAS-seq, followed by TCR clonotype profiling (FIG. 2A; FIG. 8). Here, input CD8⁺ T cells from the same source were stimulated with CMV pp65 peptide (NLVPMVATV) loaded aAPCs for two days before either Dextramer staining or ATLAS-seq, and two independent replicates were performed for each method.

From Dextramer staining, scTCR profiling detected 1547 clonotypes from 2035 T cells in replicate 1 and 1474 clonotypes from 1938 T cells in replicate 2, on average 19.5±0.21% of the clonotypes in each replicate had at least two cells (FIG. 2B). From ATLAS-seq, scTCR profiling detected 1412 clonotypes from 2070 T cells in replicate 1 and 622 clonotypes from 939 T cells in replicate 2, on average 24+0.28% of clonotypes in each replicate had at least two cells (FIG. 2B). The TCRα and TCR β genes identified using the 2 methods were largely different, only 17.2% of TCRα genes (86/500) and 18.5% of TCRβ genes (92/498) from ATLAS-seq were shared with TCRs from Dextramer staining (FIG. 2C).

After analyzing the usages of V, J segments of TCRα and TCRβ from clonotypes detected by Dextramer staining and ATLAS-seq, the inventors found they shared similar V, J segment usages. For example, among the top 10 TRAVs from each method, 8 are overlapped, which are TRAV19, TRAV21, TRAV12-2, TRAV13-1, TRAV3, TRAV14/DV4, TRAV29/DV5 and TRAV17. The top-5 used TRBVs from each method were the same TRBV27, TRBV9, TRBV7-9, TRBV28 and TRBV6-5. And the top TRBJ in each method is the same TRBJ2-7 (FIG. 2D). The similar V, J segment usages in CMV-specific TCR clonotypes were reported in previous research (Wang, Dash et al., 2012; Link, Eugster et al., 2016; Huth, Liang et al., 2019), where the above V, J segments were the major segments detected in CMV-specific CD8⁺ T cells from donors of different ages and genders.

The TCRα and TCRβ CDR3 amino acid (AA) sequences detected by Dextramer staining and ATLAS-seq were distinct, only 17.6% TCRα CDR3s (88/500) and 18.7% of TCRβ CDR3S (93/498) from ATLAS-seq were shared with TCRs from Dextramer staining (FIG. 2E). The AA length distributions from the two methods were similar (p-value >0.05 by Mann-Whitney U test) with more 14 and 15 AAs in length (FIG. 2F), which is consistent with TCRβ AA length distribution from repeated CMV reactivation patients reported in previous research (Nakasone, Kusuda et al., 2021). The inventors then use GLAM2 (Frith, Saunders et al., 2008) to identify the consensus AA sequence of TCRα and TCRβ 15 AA-long CDR3s from each method (FIG. 2G). From Dextramer staining, the inventors identified N-terminal “CAVS” and C-terminal “KLI/TF” in TCRα CDR3, N-terminal “CASS” and C-terminal “N/YEQYF” in TCR CDR3, which were also reported in CMV-specific TCR CDR3 sequences from previous research (Wang, Dash et al., 2012; Chen, Yang et al., 2017; Nakasone, Kusuda et al., 2021). The AA sequences identified from ATLAS-seq, although possess similar terminal sequences as the ones identified from Dextramer staining, the central regions are largely distinct.

In summary, the differences between CMV-specific TCR clonotypes detected by Dextramer staining and ATLSA-seq mainly result from different V-J combinations and the central regions of the CDR3 sequences, while the V, J segment usages, CDR3 lengths and the terminal regions of the CDR3 sequences were similar.

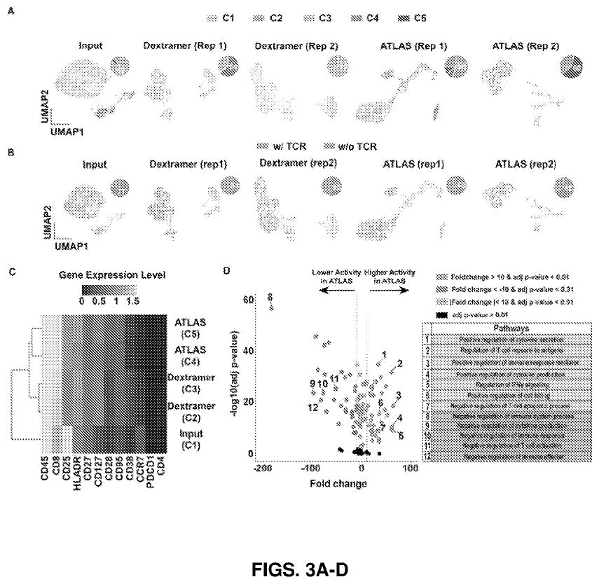

CD8⁺ T cells selected by ATLAS-seq showed higher activation levels than those selected by Dextramer staining. From the same CD8⁺ T cells for scTCR profiling, the inventors also prepared libraries for scRNA-seq and analyzed the data using 10× Genomics Cell ranger (Zheng, Terry et al., 2017) and Seurat (Hao, Hao et al., 2021)). In each of the input, Dextramer staining (n=2 replicates) and ATLAS-seq (n=2 replicates), the inventors clustered all individual CD8⁺ T cells into several distinct clusters based on their gene expressions through dimensionality reduction UMAP (FIG. 3A). They also projected the TCR clonotypes status (presence/absence) of each individual cell onto these cell clusters (FIG. 3B). In each sample, there was a major cell cluster with over 50% of total cells with TCR clonotype information. Clusters (FIG. 3A, gray) were excluded from downstream analyses due to either the lack of TCR information or the higher proportion of mitochondrial gene transcripts (filtered with Seurat, indicating low quality cells (Hao, Hao et al., 2021)).

The inventors used 12 previously identified gene markers indicated in T cell activation, differentiation, and exhaustion (De Biasi, Meschiari et al., 2020; Penkava, Velasco-Herrera et al., 2020) and calculated their normalized average expression level (Butler, Hoffman et al., 2018) to evaluate the phenotypes of CD8⁺ T cells from the major clusters of different samples (Cluster 1-5, FIG. 3A, and FIG. 3C). All clusters had negligible expression of PDCD1 gene, a key molecule responsible for T cell exhaustion and negatively regulates the activation of antigen-specific T cells (McLane, Abdel-Hakeem et al., 2019; Wherry and Kurachi 2015). The expression of CD127 gene, a marker of T cells with lower exhaustion status (McLane, Abdel-Hakeem et al., 2019; Wherry and Kurachi 2015), was significantly higher in both Dextramer staining (Cluster 2 and 3) and ATLAS-seq (Cluster 4 and 5) selected CD8⁺ T cell than in the input sample (Cluster 1). When compared with the input sample, the selected CD8⁺ T cells from both Dextramer staining and ATLAS-seq showed lower expression levels of CD25 and CCR7, which are reported as down-regulated genes in activated effector T cells (Sheu, Lin et al., 1997; De Biasi, Meschiari et al., 2020; Penkava, Velasco-Herrera et al., 2020). The opposite trend of higher expression level in the selected CD8⁺ T cells is also observed for HLADR gene, a marker for activated CD8⁺ T cells (De Biasi, Meschiari et al., 2020; Penkava, Velasco-Herrera et al., 2020) (FIG. 3C). These results suggest that T cells selected by both methods are activated without signs of exhaustion.

To compare the antigen-specific activation status between Dextramer staining and ATLAS-seq selected CD8⁺ T cells, the inventors used Seurat (Hao, Hao et al., 2021)) to integrate scRNA-seq data from both methods, then analyzed the differential immune regulation pathway activities with SCPA package (Bibby, Agarwal et al., 2022). 117 immune regulation pathways were collected from Gene Ontology (Ashburner, Ball et al., 2000), Reactome Pathway (Fabregat, Sidiropoulos et al., 2017), Human Molecular Signatures (Subramanian, Tamayo et al., 2005), WikiPathways (Martens, Ammar et al., 2021), Pathway Interaction (Schaefer, Anthony et al., 2009) and KEGG databases (Kanehisa and Goto 2000). 34 pathways had significantly higher activities and 32 pathways had significantly lower activities in ATLAS-seq selected CD8⁺ T cells than in Dextramer staining selected CD8⁺ T cells (FIG. 3D). In particular, activities of several positive regulation pathways of cytokine (especially IFNγ) production and secretion were significantly higher in ATLAS-seq selected CD8⁺ T cells (pathway #1, 3, 4, 5), and these cells also exhibit higher pathway activities of positive regulations of antigen response (pathway #2) and cell killing (pathway #6) (FIG. 3D). In contrast, several negative regulation pathways of cytokine production (pathway #9) and T cell activation (pathway #11) had significantly higher activities in CD8⁺ T cells selected by Dextramer staining (FIG. 3D). In summary, based on single-cell RNA expression profiles, ATLAS-seq was able to select specific CD8⁺ T cells with higher reactivities to an antigen peptide-MHC stimulation than the CD8⁺ T cells selected by Dextramer staining.

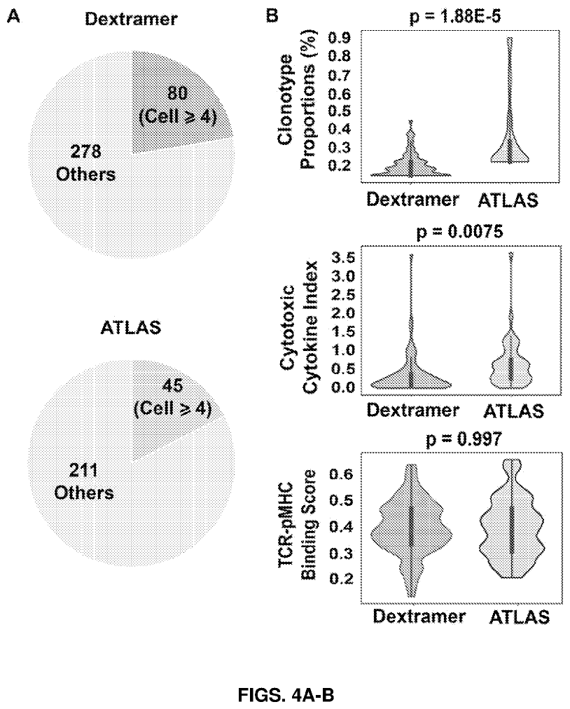

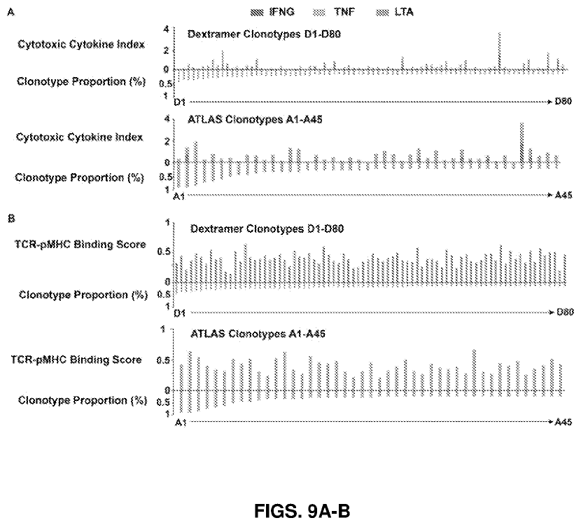

ATLASs-seq selected TCR clonotypes are more efficient for target cell killing. Next, the inventors compared the features of CMV-specific TCR clonotypes selected by Dextramer staining and ATLAS-seq to further evaluate their target-cell-killing potentials. Only CD8⁺ T cells belong to one of the major clusters (FIG. 3A) with complete a, β paired TCR information (FIG. 3B) were used in this set of analysis. They first selected clonotypes with at least 2 cells in at least one replicate of each method. A total of 358 TCR clonotypes were selected using Dextramer staining, and a total of 256 TCR clonotypes were selected using ATLAS-seq. The inventors then defined “high-priority” CMV-specific TCR clonotypes as those with at least a total of 4 cells in the two replicates of each method. They identified a total of 80 high-priority clonotypes using Dextramer staining and 45 high-priority clonotypes using ATLAS-seq (FIG. 4A). They then defined “cytotoxic cytokine index” as the normalized average RNA expression level of three important cytotoxic cytokines, including IFNg, TNFα and LTα (TNFβ) (Gocher, Workman et al., 2022; van Loo and Bertrand 2023; Tang, Zhu et al., 2017), and use it as a proxy to quantify the cytotoxicity of T cells with a specific TCR clonotype. The inventors calculated the proportions, cytotoxic cytokine indexes and TCR-pMHC binding scores of the high-priority clonotypes from each method (FIG. 4B FIGS. 9A-B). The clonotypes from ATLAS-seq showed higher proportions in selected CD8⁺ T cells and higher cytotoxic cytokine indexes than those from Dextramer staining, while no significant difference between their TCR-pMHC binding scores is observed. These results suggest an increased clonotype proportion and enrichment for higher cytotoxicity clonotypes using ATLAS-seq. This is also in concordance with the observation that cytotoxicity related cytokine production and secretion pathway activities were significantly different between Dextramer staining- and ATLAS-seq-selected cells (FIG. 3D).

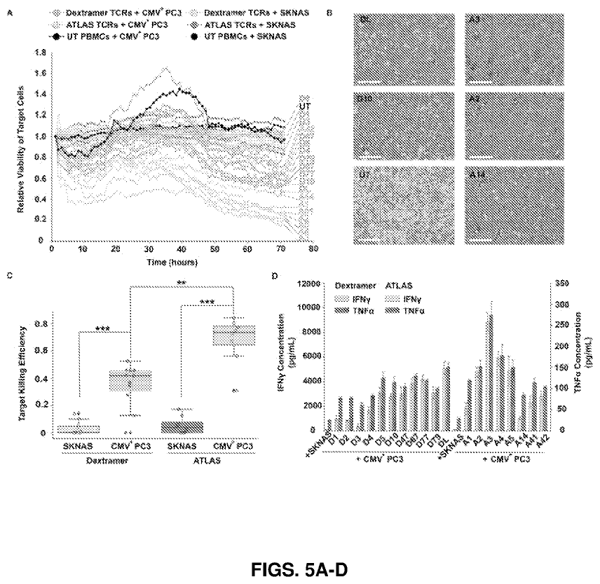

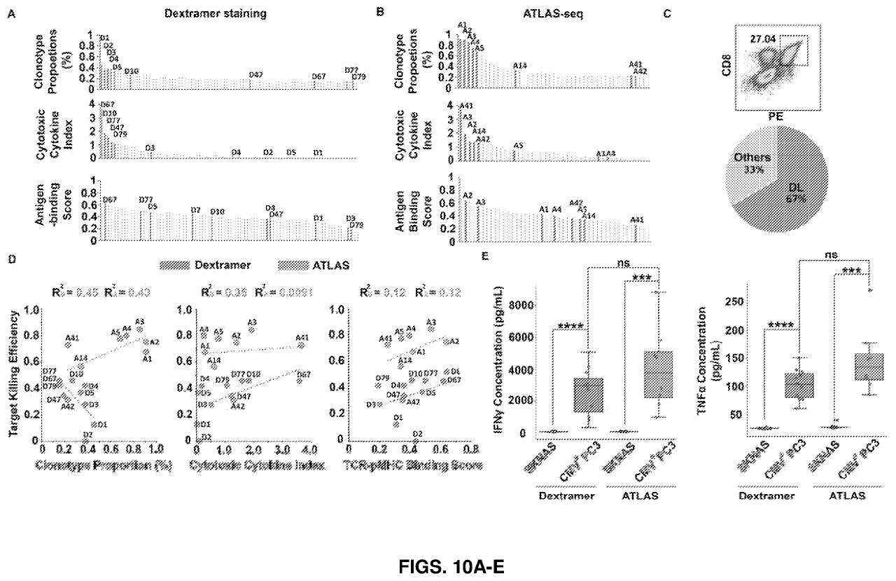

Next, the inventors measure the on-target killing potentials of select CMV-specific TCR clonotypes, they chose clonotypes with top abundances (base on clonotype proportion) or top cytotoxic cytokine indexes (based on scRNA-seq) from the two methods (Table S1). From Dextramer staining, the inventors selected the 5 most abundant clonotypes (D1, D2, D3, D4 and D5) and 5 clonotypes with the highest cytotoxic cytokine indexes (D67, D10, D77, D47 and D 79) (FIG. 10A). In ATLAS-seq, they selected the 5 most abundant clonotypes (A1, A2, A3, A4 and A5) and 5 clonotypes with the highest cytotoxic cytokine indexes (A41, A3, A2, A14, and A42) (FIG. 10B). Some clonotypes with the highest cytotoxic cytokine indexes also has the highest clonotype proportion, such as A2 and A3 (FIG. 10B). They used TCR a and β gBlocks™ Gene Fragments to clone the CMV-specific TCR clonotypes into retroviral vectors and overexpressed in CMV negative human PBMCs using RetroNectin transduction. PBMCs transduced with CMV-specific TCRs were activated by CMV pp65 peptide (NLVPMVATV (SEQ ID NO: 117)) loaded aAPC beads and cocultured for 3 days with a PC3 epithelial prostate cancer cell line that expressed GFP, HLA-A*02:01 and CMV pp65 peptide (Nesterenko, McLaughlin et al., 2021) at 10:1 PBMC: PC3 cell ratio (FIGS. 5A-B). The CMV-specific cytotoxicity was visualized by measuring loss of green fluorescence in the GFP expressing PC3 cells as relative viabilities, which shows all of the clonotypes except for A42 from ATLAS-seq had better killing performances than all of the clonotypes from Dextramer staining (FIG. 5A). There was no detectable killing observed from un-transduced (UT) PBMCs or TCR transduced PBMCs co-cultured with an unrelated SKNAS neuroblastoma cell line that does not express CMV pp65 peptide (FIG. 5A). For comparison, the inventors also tested the CMV-specific cytotoxicity of a predominant clonotype DL selected using Dextramer staining of PBMCs from the same donor but underwent a conventional long-term 2-week-antigen-stimulation (FIG. 10C). Clonotype DL's killing efficiency fell on the higher end of the range of killing efficiency of the clonotypes from Dextramer staining but is still less than most clonotypes from ATLAS-seq (FIG. 5A). TCR clonotypes with significant cytotoxicity show visible T cell clustering (Yarmarkovich, Marshall et al., 2021) and less GFP signal from the GFP⁺PC3 cells (FIG. 5B). The tested clonotypes from ATLAS-seq had significantly higher target killing efficiency (p-value=2.02E-3) than those from Dextramer staining (FIG. 5C). The inventors further measured the concentrations of secreted IFNγ and TNFα using ELISA in the conditioned media after the 3-day killing assay (FIG. 5D). PBMCs transduced with TCR clonotypes from both methods showed significant secretion of IFNγ and TNFα when co-incubated with the CMV⁺PC3 cells, this is in contrast to the co-incubation with the CMV SKNAS negative control cells. However, no significant differences in secreted IFNγ or TNFα concentration were observed between the clonotypes from the two methods (FIG. 10E). In summary, comparing to Dextramer staining, ATLAS-seq was able to enrich antigen-specific TCR clonotypes of CD8⁺ T cells that can elicit higher cytotoxicity in target cell killing.

### Example 3—Discussion

In this study, the inventors established a microfluidic-based antigen-specific single-T cell activation screening and TCR clonotype profiling workflow, which used IFN-γ secretion level as the indicator of T cell activation for selection. Single-T cell droplets generated by microfluidics provided an enclosed environment to prevent the crosstalk between T cells in their responses to antigen stimulations, allowing the precise identification of the activated T cell population. Isolated droplets can also provide a unique model system to mimic tumor microenvironments (Tsai, Trubelja et al., 2017).

In ATLAS-seq, the inventors used low input T cell concentration (0.6×10⁶ cells/mL) to achieve isolated single-T cells in droplets and resulted in ˜0.4×10⁶ CD8⁺ T cells as input in each screen. Here, the ATLAS-seq using about 0.8×10⁶ T cell as input identified 500 CMV-specific TCRα and 498 CMV-specific TCRβ clonotypes. As a reference, dextramer staining using 2×10⁶ CD8⁺ T cell input detected 608 CMV-specific TCRα and 550 CMV-specific TCRB clonotypes. A previous study using large scale Dextramer staining (Chen, Yang et al., 2017) identified an average of 593 CMV-specific TCRα and 605 CMV-specific TCRβ clonotypes, indicating a similar scale of output as the inventors' current ATLAS-seq workflow. Human CD8⁺ TCRb repertoire ranges from 10⁵-10⁸ (Li, Hiroi et al., 2016; Qi, Liu et al., 2014; Robins, Srivastava et al., 2010; Li et al., 2016; Qi et al., 2014; Robins et al., 2010), and the combined projected TCRβ repertoires against NLV (CMVpp65) were estimated to occupy up to 3.4% (0.03%-3.4%) of an adult total CD8⁺ TCRβ repertoire (Chen, Yang et al., 2017). Thus, ATLAS-seq is by no means exhaustive, but aims at selecting the most effectively activated CD8⁺ T cells from a T cell repertoire that has been properly primed with target antigen, with prior antigen exposure or pre-enriched with other methods.

By comparing the features of clonotypes selected by ATLAS-seq and Dextramer staining in this study, the inventors noticed that ATLAS-seq clonotypes showed significantly higher activation levels than Dextramer staining clonotypes, measured by pathway activities (FIG. 3D) and cytotoxic cytokine index (FIG. 4B), while there was no significant difference between their TCR-pMHC binding scores. This result suggested that high TCR-pMHC binding strength may not always be associated with high TCR activation. Since TCR-pMHC interaction is a pre-requisite for CD8⁺ T cells to exit quiescence and initiate antigen specific T-cell clonal expansion (Guy, Vignali et al., 2013; Shah, A1-Haidari et al., 2021), by selecting TCRs with multiple cell counts in either ATLAS-seq or Dextramer staining, the inventors are already selecting antigen specific TCRs that has sufficiently high affinity to the pMHC and underwent clonal expansion/proliferation before or during the screening processes, thus the similar TCR-peptide binding scores between the two methods as observed in FIG. 4B. According to previous research, the T cell activation level is maximized at intermediate TCR-pMHC affinities (Kalergis, Boucheron et al., 2001; Dushek, Aleksic et al., 2011; Corse, Gottschalk et al., 2010), which could be regulated by the fluctuations in TCR-pMHC binding dynamics and TCR phosphorylation signaling (Lever, Maini et al., 2014; Limozin, Bridge et al., 2019; Lin, Low-Nam et al., 2019). Thus, considering the complex relationship between the TCR-pMHC binding and T cell activation level, directly using T cell activation phenotypes, e.g., INFγ secretion as in ATLAS-seq, could enable a more efficient screen for functional TCRs that can be used in immunotherapy.

To narrow down the CMV-specific TCR clonotypes for in-depth evaluation of cytotoxicity, the inventors used different clonotype features including clonotype proportions, cytotoxic cytokine indexes and TCR-pMHC binding scores to rank TCR clonotype hits from the two screening methods (FIG. 9A). Here, the clonotype proportions represent the proliferation potential of activated T cells; cytotoxic cytokine indexes represent the RNA expression level of IFNγ, TNFα and LTα (TNFβ); and TCR-pMHC binding scores represent the binding strengths between TCRs and CMVpp65 peptide-MHC complex. The inventors chose clonotypes with top abundances (based on clonotype proportion) or top cytotoxic cytokine indexes (based on scRNA-seq) from the two methods. The target killing efficiency measured in the target cell killing assays using PBMC transduced with selected TCRs showed best correlation with clonotype proportions in ATLAS-seq (R²=0.43, FIG. 10D, left panel). This observation could potentially be explained by the activated T cell proliferation within the droplet during the co-incubation period in ATLAS-seq. At the end of the 2-day co-incubation period, highly activated T cells could have enough time to proliferate within the droplet. When released from the droplet after the co-incubation, the inventors will be sorting the progenies of the activated T cells. Because the cell membrane divides during mitosis, each progeny T cell could also get aptamers on its membrane. The more progeny T-cells in one droplet, the higher combined IFNγ concentration in the droplet, the higher fluorescence signal on all progeny T cells if the amount of aptamer was in excess. This fluorescent signal could plateau if progeny T cells only carry limited number of aptamers. Under this scenario, highly proliferative clonotypes will result in high clonotype proportions and high fluorescence in selected T cell population, even if such clonotype does not produce the high IFNγ. The apparent high fluorescence of the progeny T cells could be the cumulative effect of the IFNγ secreted by all the progeny T cells in the same droplet. This could at least partially explain the observation that all the clonotypes with top clonotype proportions show high target cell killing efficiency (FIG. 10, left panel), despite some clonotypes (A1 and A4) show lower cytotoxic cytokine index (FIG. 10B, middle panel). Based on these results, the inventors speculate that although cytotoxic cytokines expression is wildly used as an indicator for T cell activation and cytotoxicity, it may not be sufficient to predict the potential killing activities of the TCR clonotypes for selecting clonotypes for further testing. In the inventors' current ATLAS-seq setting, choosing the most abundant TCR clonotypes with high IFNγ aptamer fluorescent signal appears to provide good prediction for high target cell killing efficiencies, although the robustness of such correlation remains to be determined with more cloned TCR clonotypes from ATLAS-seq hits.

In addition to the interaction between CD8⁺ T cells and aAPCs in this study, other cell interactions could also be used as the basis to perform phenotype screening with ATLAS-seq platform. For example, B cell clonotypes with high antigen-specific antibody secretions could be selected based on B cell-APC interactions, or cytotoxic T cells (cytotoxic T lymphocyte, CTLs) with high infiltration abilities could be selected based on CTL-tumor cell interactions. Furthermore, cholesterol modified aptamer beacons enabled a stable labelling on cell membrane and direct probing of the extracellular environment (You, Lyu et al., 2017; Qiu, Wimmers et al., 2017). Thus, in addition to IFNγ, a set of aptamer beacons specific to different cytokine or metabolite could be prepared based on previously published aptamers (Boshtam, Asgary et al., 2017; Kim, Noh et al., 2021) to monitor different combinations of cytokine or metabolite secretions from immune cells (Shyer, Flavell et al., 2020; Heintzman, Fisher et al., 2022) in ATLAS-seq, which could potentially increase the resolution of the phenotype selection.

In conclusion, ATLAS-seq method provides an efficient workflow to screen for highly reactive antigen-specific TCR clonotypes based on T cell activation signature, and through direct monitoring of the secretion of the cytotoxic cytokine INFγ. The inventors expect this method to facilitate the discovery of highly functional TCRs for cancer TCR therapy.

TABLE 1A
Select TCR Clonotypes from Dextramer Staining

| | | | SEQ |
|---|---|---|---|
| TCR | Chain | gBlock Sequences | ID NO |
| D1 | α | CCCTCACTCCTTCTCTAGGCGCCGGAATTCAGATCTAC | 1 |
| | | Catgacatccattcgagctgtatttatattcctgtggctgcagctggacttggtgaat | |
| | | ggagagaatgtggagcagcatccttcaaccctgagtgtccaggagggagaca | |
| | | gcgctgttatcaagtgtacttattcagacagtgcctcaaactacttcccttggtataa | |
| | | gcaagaacttggaaaaggacctcagcttattatagacattcgttcaaatgtgggc | |
| | | gaaaagaaagaccaacgaattgctgttacattgaacaagacagccaaacattt | |
| | | ctccctgcacatcacagagacccaacctgaagactcggctgtctacttctgtgca | |
| | | gcaagtTGTGCAGCAAGTAATACCTATGGGAACAACAGA | |
| | | CTCGCTTTTaatatccagaaccctgaccctgccgtgtaccagctgagaga | |
| | | ctctaaatccagtgacaagtctgtctgcctattcaccgattttgattctcaaacaaat | |
| | | gtgtcacaaagtaaggattctgatgtgtatatcacagacaaaactgtgctagacat | |
| | | gaggtctatggacttcaagagcaacagtgctgtggcctggagcaacaaatctga | |
| | | ctttgcatgtgcaaacgccttcaacaacagcattattccagaagacaccttcttccc | |
| | | cagcccagaaagttcctgtgatgtcaagctggtcgagaaaagctttgaaacaga | |
| | | tacgaacctaaactttcaaaacctgtcagtgattgggttccgaatcctcctcctgaa | |
| | | agtggccgggtttaatctgctcatgacgctgcggctgtggtccagcGGCTCC | |
| | | GGAGCCACGAACTTCTCTCTGTTAAAGCAAGCAGGAGA | |
| | | CGTGGAAGAAAACCCCGGTCCC | |
| | | GCAGGAGACGTGGAAGAAAACCCCGGTCCCatgggctcca | |
| | β | ggctgctctgttgggtgctgctttgtctcctgggagcaggcccagtaaaggctgga | 2 |
| | | gtcactcaaactccaagatatctgatcaaaacgagaggacagcaagtgacact | |
| | | gagctgctcccctatctctgggcataggagtgtatcctggtaccaacagacccca | |
| | | ggacagggccttcagttcctctttgaatacttcagtgagacacagagaaacaaag | |
| | | gaaacttccctggtcgattctcagggcgccagttctctaactctcgctctgagatga | |
| | | atgtgagcaccttggagctgggggactcggccctttatctttgcgccagcagcttg | |
| | | TGCGCCAGCAGCCTAAAACGGGGGCTGACAGATACGC | |
| | | AGTATTTTgaggacctgaaaaacgtgttcccacccgaggtcgctgtgtttga | |
| | | gccatcagaagcagagatctcccacacccaaaaggccacactggtgtgcctgg | |
| | | ccacaggcttctaccccgaccacgtggagctgagctggtgggtgaatgggaag | |
| | | gaggtgcacagtggggtcagcacagacccgcagcccctcaaggagcagccc | |
| | | gccctcaatgactccagatactgcctgagcagccgcctgagggtctcggccacc | |
| | | ttctggcagaacccccgcaaccacttccgctgtcaagtccagttctacgggctctc | |
| | | ggagaatgacgagtggacccaggatagggccaaacctgtcacccagatcgtc | |
| | | agcgccgaggcctggggtagagcagactgtggcttcacctccgagtcttaccag | |
| | | caaggggtcctgtctgccaccatcctctatgagatcttgctagggaaggccacctt | |
| | | gtatgccgtgctggtcagtgccctcgtgctgatggccatggtcaagagaaaggatt | |
| | | ccagaggcTGACTCGAGCGGGATCAATTCCGCCCCCCCC | |
| | | CT | |
| D2 | α | CCCTCACTCCTTCTCTAGGCGCCGGAATTCAGATCTAC | 3 |
| | | Catgtggggagttttccttctttatgtttccatgaagatgggaggcactacaggaca | |
| | | aaacattgaccagcccactgagatgacagctacggaaggtgccattgtccagat | |
| | | caactgcacgtaccagacatctgggttcaacgggctgttctggtaccagcaacat | |
| | | gctggcgaagcacccacatttctgtcttacaatgttctggatggtttggaggagaa | |
| | | aggtcgtttttcttcattccttagtcggtctaaagggtacagttacctccttttgaagga | |
| | | gctccagatgaaagactctgcctcttacctctgtgctgtgagaTGTGCTGTC | |
| | | CATTGGATGGATAGCAGCTATAAATTGATCTTCaatatcca | |
| | | gaaccctgaccctgccgtgtaccagctgagagactctaaatccagtgacaagtct | |
| | | gtctgcctattcaccgattttgattctcaaacaaatgtgtcacaaagtaaggattctg | |
| | | atgtgtatatcacagacaaaactgtgctagacatgaggtctatggacttcaagag | |
| | | caacagtgctgtggcctggagcaacaaatctgactttgcatgtgcaaacgccttc | |
| | | aacaacagcattattccagaagacaccttcttccccagcccagaaagttcctgtg | |
| | | atgtcaagctggtcgagaaaagctttgaaacagatacgaacctaaactttcaaa | |
| | | acctgtcagtgattgggttccgaatcctcctcctgaaagtggccgggtttaatctgct | |
| | | catgacgctgcggctgtggtccagcGGCTCCGGAGCCACGAACTT | |
| | | CTCTCTGTTAAAGCAAGCAGGAGACGTGGAAGAAAACC | |
| | | CCGGTCCC | |
| | β | GCAGGAGACGTGGAAGAAAACCCCGGTCCCatggcctccct | 4 |
| | | gctcttcttctgtggggccttttatctcctgggaacagggtccatggatgctgatgtta | |
| | | cccagaccccaaggaataggatcacaaagacaggaaagaggattatgctgg | |
| | | aatgttctcagactaagggtcatgatagaatgtactggtatcgacaagacccagg | |
| | | actgggcctacagttgatctattactcctttgatgtcaaagatataaacaaaggag | |
| | | agatctctgatggatacagtgtctctcgacaggcacaggctaaattctccctgtccc | |
| | | tagagtctgccatccccaaccagacagctctttacttctgtgccaccagtgatttgT | |
| | | GTGCCACCAGTGACCGGCAGGGGTACAATGAGCAGTT | |
| | | CTTCgaggacctgaaaaacgtgttcccacccgaggtcgctgtgtttgagccat | |
| | | cagaagcagagatctcccacacccaaaaggccacactggtgtgcctggccac | |
| | | aggcttctaccccgaccacgtggagctgagctggtgggtgaatgggaaggagg | |
| | | tgcacagtggggtcagcacagacccgcagcccctcaaggagcagcccgccct | |
| | | caatgactccagatactgcctgagcagccgcctgagggtctcggccaccttctgg | |
| | | cagaacccccgcaaccacttccgctgtcaagtccagttctacgggctctcggag | |
| | | aatgacgagtggacccaggatagggccaaacctgtcacccagatcgtcagcg | |
| | | ccgaggcctggggtagagcagactgtggcttcacctccgagtcttaccagcaag | |
| | | gggtcctgtctgccaccatcctctatgagatcttgctagggaaggccaccttgtatg | |
| | | ccgtgctggtcagtgccctcgtgctgatggccatggtcaagagaaaggattccag | |
| | | aggcTGACTCGAGCGGGATCAATTCCGCCCCCCCCCT | |
| D3 | α | CCCTCACTCCTTCTCTAGGCGCCGGAATTCAGATCTAC | 5 |
| | | Catggcatgccctggcttcctgtgggcacttgtgatctccacctgtcttgaatttagc | |
| | | atggctcagacagtcactcagtctcaaccagagatgtctgtgcaggaggcagag | |
| | | accgtgaccctgagctgcacatatgacaccagtgagagtgattattatttattctggt | |
| | | acaagcagcctcccagcaggcagatgattctcgttattcgccaagaagcttataa | |
| | | gcaacagaatgcaacagagaatcgtttctctgtgaacttccagaaagcagccaa | |
| | | atccttcagtctcaagatctcagactcacagctgggggatgccgcgatgtatttctg | |
| | | tgcttataggagcTGTGCTTATAGGCCCTCTTATTCAGGAAAC | |
| | | ACACCTCTTGTCTTTaatatccagaaccctgaccctgccgtgtaccagc | |
| | | tgagagactctaaatccagtgacaagtctgtctgcctattcaccgattttgattctca | |
| | | aacaaatgtgtcacaaagtaaggattctgatgtgtatatcacagacaaaactgtg | |
| | | ctagacatgaggtctatggacttcaagagcaacagtgctgtggcctggagcaac | |
| | | aaatctgactttgcatgtgcaaacgccttcaacaacagcattattccagaagaca | |
| | | ccttcttccccagcccagaaagttcctgtgatgtcaagctggtcgagaaaagcttt | |
| | | gaaacagatacgaacctaaactttcaaaacctgtcagtgattgggttccgaatcct | |
| | | cctcctgaaagtggccgggtttaatctgctcatgacgctgcggctgtggtccagcG | |
| | | GCTCCGGAGCCACGAACTTCTCTCTGTTAAAGCAAGCA | |
| | | GGAGACGTGGAAGAAAACCCCGGTCCC | |
| | β | GCAGGAGACGTGGAAGAAAACCCCGGTCCCatgggctgca | 6 |
| | | ggctgctctgctgtgcggttctctgtctcctgggagcagttcccatagacactgaag | |
| | | ttacccagacaccaaaacacctggtcatgggaatgacaaataagaagtctttga | |
| | | aatgtgaacaacatatggggcacagggctatgtattggtacaagcagaaagcta | |
| | | agaagccaccggagctcatgtttgtctacagctatgagaaactctctataaatgaa | |
| | | agtgtgccaagtcgcttctcacctgaatgccccaacagctctctcttaaaccttcac | |
| | | ctacacgccctgcagccagaagactcagccctgtatctctgcgccagcagccaa | |
| | | TGCGCCAGCAGCCTACGGACCAATACCTACGAGCAGT | |
| | | ACTTCgaggacctgaaaaacgtgttcccacccgaggtcgctgtgtttgagcca | |
| | | tcagaagcagagatctcccacacccaaaaggccacactggtgtgcctggccac | |
| | | aggcttctaccccgaccacgtggagctgagctggtgggtgaatgggaaggagg | |
| | | tgcacagtggggtcagcacagacccgcagcccctcaaggagcagcccgccct | |
| | | caatgactccagatactgcctgagcagccgcctgagggtctcggccaccttctgg | |
| | | cagaacccccgcaaccacttccgctgtcaagtccagttctacgggctctcggag | |
| | | aatgacgagtggacccaggatagggccaaacctgtcacccagatcgtcagcg | |
| | | ccgaggcctggggtagagcagactgtggcttcacctccgagtcttaccagcaag | |
| | | gggtcctgtctgccaccatcctctatgagatcttgctagggaaggccaccttgtatg | |
| | | ccgtgctggtcagtgccctcgtgctgatggccatggtcaagagaaaggattccag | |
| | | aggcTGACTCGAGCGGGATCAATTCCGCCCCCCCCCT | |
| D4 | α | CCCTCACTCCTTCTCTAGGCGCCGGAATTCAGATCTAC | 7 |
| | | Catggagaagaatcctttggcagccccattactaatcctctggtttcatcttgactgc | |
| | | gtgagcagcatactgaacgtggaacaaagtcctcagtcactgcatgttcaggag | |
| | | ggagacagcaccaatttcacctgcagcttcccttccagcaatttttatgccttacact | |
| | | ggtacagatgggaaactgcaaaaagccccgaggccttgtttgtaatgactttaaa | |
| | | tggggatgaaaagaagaaaggacgaataagtgccactcttaataccaaggag | |
| | | ggttacagctatttgtacatcaaaggatcccagcctgaagactcagccacatacc | |
| | | tctgtgcctttTGTGCCTCCCAGGGCACTAACTTTGGAAATGA | |
| | | GAAATTAACCTTTaatatccagaaccctgaccctgccgtgtaccagctga | |
| | | gagactctaaatccagtgacaagtctgtctgcctattcaccgattttgattctcaaac | |
| | | aaatgtgtcacaaagtaaggattctgatgtgtatatcacagacaaaactgtgctag | |
| | | acatgaggtctatggacttcaagagcaacagtgctgtggcctggagcaacaaat | |
| | | ctgactttgcatgtgcaaacgccttcaacaacagcattattccagaagacaccttct | |
| | | tccccagcccagaaagttcctgtgatgtcaagctggtcgagaaaagctttgaaac | |
| | | agatacgaacctaaactttcaaaacctgtcagtgattgggttccgaatcctcctcct | |
| | | gaaagtggccgggtttaatctgctcatgacgctgcggctgtggtccagcGGCT | |
| | | CCGGAGCCACGAACTTCTCTCTGTTAAAGCAAGCAGGA | |
| | | GACGTGGAAGAAAACCCCGGTCCC | |
| | β | GCAGGAGACGTGGAAGAAAACCCCGGTCCCatgggccccc | 8 |
| | | agctccttggctatgtggtcctttgccttctaggagcaggccccctggaagcccaa | |
| | | gtgacccagaacccaagatacctcatcacagtgactggaaagaagttaacagt | |
| | | gacttgttctcagaatatgaaccatgagtatatgtcctggtatcgacaagacccag | |
| | | ggctgggcttaaggcagatctactattcaatgaatgttgaggtgactgataaggga | |
| | | gatgttcctgaagggtacaaagtctctcgaaaagagaagaggaatttccccctg | |
| | | atcctggagtcgcccagccccaaccagacctctctgtacttctgtgccagcagttt | |
| | | aTGTGCCAGCAGTTTATATGGGGGAAACACTGAAGCTT | |
| | | TCTTTgaggacctgaacaaggtgttcccacccgaggtcgctgtgtttgagcca | |
| | | tcagaagcagagatctcccacacccaaaaggccacactggtgtgcctggccac | |
| | | aggcttcttccccgaccacgtggagctgagctggtgggtgaatgggaaggaggt | |
| | | gcacagtggggtcagcacggacccgcagcccctcaaggagcagcccgccctc | |
| | | aatgactccagatactgcctgagcagccgcctgagggtctcggccaccttctggc | |
| | | agaacccccgcaaccacttccgctgtcaagtccagttctacgggctctcggaga | |
| | | atgacgagtggacccaggatagggccaaacccgtcacccagatcgtcagcgc | |
| | | cgaggcctggggtagagcagactgtggctttacctcggtgtcctaccagcaagg | |
| | | ggtcctgtctgccaccatcctctatgagatcctgctagggaaggccaccctgtatg | |
| | | ctgtgctggtcagcgcccttgtgttgatggccatggtcaagagaaaggatttcTG | |
| | | ACTCGAGCGGGATCAATTCCGCCCCCCCCCT | |
| D5 | α | CCCTCACTCCTTCTCTAGGCGCCGGAATTCAGATCTAC | 9 |
| | | Catggcctctgcacccatctcgatgcttgcgatgctcttcacattgagtgggctgag | |
| | | agctcagtcagtggctcagccggaagatcaggtcaacgttgctgaagggaatcc | |
| | | tctgactgtgaaatgcacctattcagtctctggaaacccttatcttttttggtatgttcaa | |
| | | taccccaaccgaggcctccagttccttctgaaatacatcacaggggataacctgg | |
| | | ttaaaggcagctatggctttgaagctgaatttaacaagagccaaacctccttccac | |
| | | ctgaagaaaccatctgcccttgtgagcgactccgctttgtacttctgtgctgtgaga | |
| | | gacTGTGCTGTGAGAGACGAAAATTCAGGATACAGCAC | |
| | | CCTCACCTTTaatatccagaaccctgaccctgccgtgtaccagctgagag | |
| | | actctaaatccagtgacaagtctgtctgcctattcaccgattttgattctcaaacaaa | |
| | | tgtgtcacaaagtaaggattctgatgtgtatatcacagacaaaactgtgctagaca | |
| | | tgaggtctatggacttcaagagcaacagtgctgtggcctggagcaacaaatctg | |
| | | actttgcatgtgcaaacgccttcaacaacagcattattccagaagacaccttcttcc | |
| | | ccagcccagaaagttcctgtgatgtcaagctggtcgagaaaagctttgaaacag | |
| | | atacgaacctaaactttcaaaacctgtcagtgattgggttccgaatcctcctcctga | |
| | | aagtggccgggtttaatctgctcatgacgctgcggctgtggtccagcGGCTCC | |
| | | GGAGCCACGAACTTCTCTCTGTTAAAGCAAGCAGGAGA | |
| | | CGTGGAAGAAAACCCCGGTCCC | |
| | β | GCAGGAGACGTGGAAGAAAACCCCGGTCCCatgggcacc | 10 |
| | | aggctcctctgctgggcggccctctgtctcctgggagcagaactcacagaagctg | |
| | | gagttgcccagtctcccagatataagattatagagaaaaggcagagtgtggctttt | |
| | | tggtgcaatcctatatctggccatgctaccctttactggtaccagcagatcctggga | |
| | | cagggcccaaagcttctgattcagtttcagaataacggtgtagtggatgattcaca | |
| | | gttgcctaaggatcgattttctgcagagaggctcaaaggagtagactccactctca | |
| | | agatccagcctgcaaagcttgaggactcggccgtgtatctctgtgccagcagctt | |
| | | aTGTGCCAGCAGCTTAAAGGACAGCTATAATGAAAAAC | |
| | | TGTTTTTTgaggacctgaacaaggtgttcccacccgaggtcgctgtgtttga | |
| | | gccatcagaagcagagatctcccacacccaaaaggccacactggtgtgcctgg | |
| | | ccacaggcttcttccccgaccacgtggagctgagctggtgggtgaatgggaagg | |
| | | aggtgcacagtggggtcagcacggacccgcagcccctcaaggagcagcccg | |
| | | ccctcaatgactccagatactgcctgagcagccgcctgagggtctcggccacctt | |
| | | ctggcagaacccccgcaaccacttccgctgtcaagtccagttctacgggctctcg | |
| | | gagaatgacgagtggacccaggatagggccaaacccgtcacccagatcgtca | |
| | | gcgccgaggcctggggtagagcagactgtggctttacctcggtgtcctaccagc | |
| | | aaggggtcctgtctgccaccatcctctatgagatcctgctagggaaggccaccct | |
| | | gtatgctgtgctggtcagcgcccttgtgttgatggccatggtcaagagaaaggattt | |
| | | CTGACTCGAGCGGGATCAATTCCGCCCCCCCCCT | |
| D10 | α | CCCTCACTCCTTCTCTAGGCGCCGGAATTCAGATCTAC | 11 |
| | | Catggcctctgcacccatctcgatgcttgcgatgctcttcacattgagtgggctgag | |
| | | agctcagtcagtggctcagccggaagatcaggtcaacgttgctgaagggaatcc | |
| | | tctgactgtgaaatgcacctattcagtctctggaaacccttatcttttttggtatgttcaa | |
| | | taccccaaccgaggcctccagttccttctgaaatacatcacaggggataacctgg | |
| | | ttaaaggcagctatggctttgaagctgaatttaacaagagccaaacctccttccac | |
| | | ctgaagaaaccatctgcccttgtgagcgactccgctttgtacttctgtgctgtgaga | |
| | | gacTGTGCTGTGTTCCCCTCTAACGACTACAAGCTCAGC | |
| | | TTTaatatccagaaccctgaccctgccgtgtaccagctgagagactctaaatcc | |
| | | agtgacaagtctgtctgcctattcaccgattttgattctcaaacaaatgtgtcacaaa | |
| | | gtaaggattctgatgtgtatatcacagacaaaactgtgctagacatgaggtctatg | |
| | | gacttcaagagcaacagtgctgtggcctggagcaacaaatctgactttgcatgtg | |
| | | caaacgccttcaacaacagcattattccagaagacaccttcttccccagcccag | |
| | | aaagttcctgtgatgtcaagctggtcgagaaaagctttgaaacagatacgaacct | |
| | | aaactttcaaaacctgtcagtgattgggttccgaatcctcctcctgaaagtggccg | |
| | | ggtttaatctgctcatgacgctgcggctgtggtccagcGGCTCCGGAGCC | |
| | | ACGAACTTCTCTCTGTTAAAGCAAGCAGGAGACGTGGA | |
| | | AGAAAACCCCGGTCCC | |
| | β | GCAGGAGACGTGGAAGAAAACCCCGGTCCCatggcctccct | 12 |
| | | gctcttcttctgtggggccttttatctcctgggaacagggtccatggatgctgatgtta | |
| | | cccagaccccaaggaataggatcacaaagacaggaaagaggattatgctgg | |
| | | aatgttctcagactaagggtcatgatagaatgtactggtatcgacaagacccagg | |
| | | actgggcctacagttgatctattactcctttgatgtcaaagatataaacaaaggag | |
| | | agatctctgatggatacagtgtctctcgacaggcacaggctaaattctccctgtccc | |
| | | tagagtctgccatccccaaccagacagctctttacttctgtgccaccagtgatttgT | |
| | | GTGCCACCAGTGATCCGGGGAGGGCTTACAATCAGCC | |
| | | CCAGCATTTTgaggacctgaacaaggtgttcccacccgaggtcgctgtgtt | |
| | | tgagccatcagaagcagagatctcccacacccaaaaggccacactggtgtgcc | |
| | | tggccacaggcttcttccccgaccacgtggagctgagctggtgggtgaatggga | |
| | | aggaggtgcacagtggggtcagcacggacccgcagcccctcaaggagcagc | |
| | | ccgccctcaatgactccagatactgcctgagcagccgcctgagggtctcggcca | |
| | | ccttctggcagaacccccgcaaccacttccgctgtcaagtccagttctacgggctc | |
| | | tcggagaatgacgagtggacccaggatagggccaaacccgtcacccagatcg | |
| | | tcagcgccgaggcctggggtagagcagactgtggctttacctcggtgtcctacca | |
| | | gcaaggggtcctgtctgccaccatcctctatgagatcctgctagggaaggccacc | |
| | | ctgtatgctgtgctggtcagcgcccttgtgttgatggccatggtcaagagaaagga | |
| | | tttcTGACTCGAGCGGGATCAATTCCGCCCCCCCCCT | |
| D47 | α | CCCTCACTCCTTCTCTAGGCGCCGGAATTCAGATCTAC | 13 |
| | | Catggaaactctcctgggagtgtctttggtgattctatggcttcaactggctagggtg | |
| | | aacagtcaacagggagaagaggatcctcaggccttgagcatccaggagggtg | |
| | | aaaatgccaccatgaactgcagttacaaaactagtataaacaatttacagtggta | |
| | | tagacaaaattcaggtagaggccttgtccacctaattttaatacgttcaaatgaaa | |
| | | gagagaaacacagtggaagattaagagtcacgcttgacacttccaagaaaag | |
| | | cagttccttgttgatcacggcttcccgggcagcagacactgcttcttacttctgtgcta | |
| | | cggacTGTGCCTACGTCAGAGATGACAAGATCATCTTTaa | |
| | | tatccagaaccctgaccctgccgtgtaccagctgagagactctaaatccagtgac | |
| | | aagtctgtctgcctattcaccgattttgattctcaaacaaatgtgtcacaaagtaagg | |
| | | attctgatgtgtatatcacagacaaaactgtgctagacatgaggtctatggacttca | |
| | | agagcaacagtgctgtggcctggagcaacaaatctgactttgcatgtgcaaacg | |
| | | ccttcaacaacagcattattccagaagacaccttcttccccagcccagaaagttc | |
| | | ctgtgatgtcaagctggtcgagaaaagctttgaaacagatacgaacctaaacttt | |
| | | caaaacctgtcagtgattgggttccgaatcctcctcctgaaagtggccgggtttaat | |
| | | ctgctcatgacgctgcggctgtggtccagcGGCTCCGGAGCCACGAA | |
| | | CTTCTCTCTGTTAAAGCAAGCAGGAGACGTGGAAGAAA | |
| | | ACCCCGGTCCC | |
| | β | GCAGGAGACGTGGAAGAAAACCCCGGTCCCatggactcct | 14 |
| | | ggaccttctgctgtgtgtccctttgcatcctggtagcgaagcatacagatgctggag | |
| | | ttatccagtcaccccgccatgaggtgacagagatgggacaagaagtgactctga | |
| | | gatgtaaaccaatttcaggccacaactcccttttctggtacagacagaccatgatg | |
| | | cggggactggagttgctcatttactttaacaacaacgttccgatagatgattcagg | |
| | | gatgcccgaggatcgattctcagctaagatgcctaatgcatcattctccactctga | |
| | | agatccagccctcagaacccagggactcagctgtgtacttctgtgccagcagttta | |
| | | TGTGCCAGCAGTTTAGCGTCAGGGGGGAATTCACCCC | |
| | | TCCACTTTgaggacctgaacaaggtgttcccacccgaggtcgctgtgtttga | |
| | | gccatcagaagcagagatctcccacacccaaaaggccacactggtgtgcctgg | |
| | | ccacaggcttcttccccgaccacgtggagctgagctggtgggtgaatgggaagg | |
| | | aggtgcacagtggggtcagcacggacccgcagcccctcaaggagcagcccg | |
| | | ccctcaatgactccagatactgcctgagcagccgcctgagggtctcggccacctt | |
| | | ctggcagaacccccgcaaccacttccgctgtcaagtccagttctacgggctctcg | |
| | | gagaatgacgagtggacccaggatagggccaaacccgtcacccagatcgtca | |
| | | gcgccgaggcctggggtagagcagactgtggctttacctcggtgtcctaccagc | |
| | | aaggggtcctgtctgccaccatcctctatgagatcctgctagggaaggccaccct | |
| | | gtatgctgtgctggtcagcgcccttgtgttgatggccatggtcaagagaaaggattt | |
| | | CTGACTCGAGCGGGATCAATTCCGCCCCCCCCCT | |
| D67 | α | CCCTCACTCCTTCTCTAGGCGCCGGAATTCAGATCTAC | 15 |
| | | Catggagaagaatcctttggcagccccattactaatcctctggtttcatcttgactgc | |
| | | gtgagcagcatactgaacgtggaacaaagtcctcagtcactgcatgttcaggag | |
| | | ggagacagcaccaatttcacctgcagcttcccttccagcaatttttatgccttacact | |
| | | ggtacagatgggaaactgcaaaaagccccgaggccttgtttgtaatgactttaaa | |
| | | tggggatgaaaagaagaaaggacgaataagtgccactcttaataccaaggag | |
| | | ggttacagctatttgtacatcaaaggatcccagcctgaagactcagccacatacc | |
| | | tctgtgcctttTGTGCCTTCCCTTACAATAACAATGACATGCG | |
| | | CTTTaatatccagaaccctgaccctgccgtgtaccagctgagagactctaaat | |
| | | ccagtgacaagtctgtctgcctattcaccgattttgattctcaaacaaatgtgtcaca | |
| | | aagtaaggattctgatgtgtatatcacagacaaaactgtgctagacatgaggtcta | |
| | | tggacttcaagagcaacagtgctgtggcctggagcaacaaatctgactttgcatgt | |
| | | gcaaacgccttcaacaacagcattattccagaagacaccttcttccccagccca | |
| | | gaaagttcctgtgatgtcaagctggtcgagaaaagctttgaaacagatacgaac | |
| | | ctaaactttcaaaacctgtcagtgattgggttccgaatcctcctcctgaaagtggcc | |
| | | gggtttaatctgctcatgacgctgcggctgtggtccagcGGCTCCGGAGC | |
| | | CACGAACTTCTCTCTGTTAAAGCAAGCAGGAGACGTGG | |
| | | AAGAAAACCCCGGTCCC | |
| | β | GCAGGAGACGTGGAAGAAAACCCCGGTCCCatgggccccc | 16 |
| | | agctccttggctatgtggtcctttgccttctaggagcaggccccctggaagcccaa | |
| | | gtgacccagaacccaagatacctcatcacagtgactggaaagaagttaacagt | |
| | | gacttgttctcagaatatgaaccatgagtatatgtcctggtatcgacaagacccag | |
| | | ggctgggcttaaggcagatctactattcaatgaatgttgaggtgactgataaggga | |
| | | gatgttcctgaagggtacaaagtctctcgaaaagagaagaggaatttccccctg | |
| | | atcctggagtcgcccagccccaaccagacctctctgtacttctgtgccagcagttt | |
| | | aTGTGCCAGCAGTTTAGAGGGTTACACTGAAGCTTTCT | |
| | | TTgaggacctgaacaaggtgttcccacccgaggtcgctgtgtttgagccatcag | |
| | | aagcagagatctcccacacccaaaaggccacactggtgtgcctggccacagg | |
| | | cttcttccccgaccacgtggagctgagctggtgggtgaatgggaaggaggtgca | |
| | | cagtggggtcagcacggacccgcagcccctcaaggagcagcccgccctcaat | |
| | | gactccagatactgcctgagcagccgcctgagggtctcggccaccttctggcag | |
| | | aacccccgcaaccacttccgctgtcaagtccagttctacgggctctcggagaatg | |
| | | acgagtggacccaggatagggccaaacccgtcacccagatcgtcagcgccga | |
| | | ggcctggggtagagcagactgtggctttacctcggtgtcctaccagcaaggggtc | |
| | | ctgtctgccaccatcctctatgagatcctgctagggaaggccaccctgtatgctgtg | |
| | | ctggtcagcgcccttgtgttgatggccatggtcaagagaaaggatttcTGACT | |
| | | CGAGCGGGATCAATTCCGCCCCCCCCCT | |
| D77 | α | CCCTCACTCCTTCTCTAGGCGCCGGAATTCAGATCTAC | 17 |
| | | Catggaaactctcctgggagtgtctttggtgattctatggcttcaactggctagggtg | |
| | | aacagtcaacagggagaagaggatcctcaggccttgagcatccaggagggtg | |
| | | aaaatgccaccatgaactgcagttacaaaactagtataaacaatttacagtggta | |
| | | tagacaaaattcaggtagaggccttgtccacctaattttaatacgttcaaatgaaa | |
| | | gagagaaacacagtggaagattaagagtcacgcttgacacttccaagaaaag | |
| | | cagttccttgttgatcacggcttcccgggcagcagacactgcttcttacttctgtgcta | |
| | | cggacTGTGCCCTGAACAGAGATGACAAGATCATCTTTaa | |
| | | tatccagaaccctgaccctgccgtgtaccagctgagagactctaaatccagtgac | |
| | | aagtctgtctgcctattcaccgattttgattctcaaacaaatgtgtcacaaagtaagg | |
| | | attctgatgtgtatatcacagacaaaactgtgctagacatgaggtctatggacttca | |
| | | agagcaacagtgctgtggcctggagcaacaaatctgactttgcatgtgcaaacg | |
| | | ccttcaacaacagcattattccagaagacaccttcttccccagcccagaaagttc | |
| | | ctgtgatgtcaagctggtcgagaaaagctttgaaacagatacgaacctaaacttt | |
| | | caaaacctgtcagtgattgggttccgaatcctcctcctgaaagtggccgggtttaat | |
| | | ctgctcatgacgctgcggctgtggtccagcGGCTCCGGAGCCACGAA | |
| | | CTTCTCTCTGTTAAAGCAAGCAGGAGACGTGGAAGAAA | |
| | | ACCCCGGTCCC | |
| | β | GCAGGAGACGTGGAAGAAAACCCCGGTCCCatgggcacc | 18 |
| | | agtctcctatgctgggtggtcctgggtttcctagggacagatcacacaggtgctgg | |
| | | agtctcccagtctcccaggtacaaagtcacaaagaggggacaggatgtagctct | |
| | | caggtgtgatccaatttcgggtcatgtatccctttattggtaccgacaggccctggg | |
| | | gcagggcccagagtttctgacttacttcaattatgaagcccaacaagacaaatca | |
| | | gggctgcccaatgatcggttctctgcagagaggcctgagggatccatctccactct | |
| | | gacgatccagcgcacagagcagcgggactcggccatgtatcgctgtgccagca | |
| | | gcttaTGTGCCAGCAGCTTAGCCGGGACAGGGCCGACC | |
| | | GGGGAGCTGTTTTTTgaggacctgaaaaacgtgttcccacccgaggt | |
| | | cgctgtgtttgagccatcagaagcagagatctcccacacccaaaaggccacact | |
| | | ggtgtgcctggccacaggcttctaccccgaccacgtggagctgagctggtgggt | |
| | | gaatgggaaggaggtgcacagtggggtcagcacagacccgcagcccctcaa | |
| | | ggagcagcccgccctcaatgactccagatactgcctgagcagccgcctgaggg | |
| | | tctcggccaccttctggcagaacccccgcaaccacttccgctgtcaagtccagttc | |
| | | tacgggctctcggagaatgacgagtggacccaggatagggccaaacctgtcac | |
| | | ccagatcgtcagcgccgaggcctggggtagagcagactgtggcttcacctccga | |
| | | gtcttaccagcaaggggtcctgtctgccaccatcctctatgagatcttgctaggga | |
| | | aggccaccttgtatgccgtgctggtcagtgccctcgtgctgatggccatggtcaag | |
| | | agaaaggattccagaggcTGACTCGAGCGGGATCAATTCCGC | |
| | | CCCCCCCCT | |
| D79 | α | CCCTCACTCCTTCTCTAGGCGCCGGAATTCAGATCTAC | 19 |
| | | Catgctcctgctgctcgtcccagtgctcgaggtgatttttaccctgggaggaacca | |
| | | gagcccagtcggtgacccagcttggcagccacgtctctgtctctgaaggagccct | |
| | | ggttctgctgaggtgcaactactcatcgtctgttccaccatatctcttctggtatgtgc | |
| | | aataccccaaccaaggactccagcttctcctgaagtacacatcagcggccaccc | |
| | | tggttaaaggcatcaacggttttgaggctgaatttaagaagagtgaaacctccttc | |
| | | cacctgacgaaaccctcagcccatatgagcgacgcggctgagtacttctgtgctg | |
| | | tgagtTGTGCTGTGAGTGTCACGGGAAGGCAGGGAGGAA | |
| | | AGCTTATCTTCaatatccagaaccctgaccctgccgtgtaccagctgaga | |
| | | gactctaaatccagtgacaagtctgtctgcctattcaccgattttgattctcaaacaa | |
| | | atgtgtcacaaagtaaggattctgatgtgtatatcacagacaaaactgtgctagac | |
| | | atgaggtctatggacttcaagagcaacagtgctgtggcctggagcaacaaatct | |
| | | gactttgcatgtgcaaacgccttcaacaacagcattattccagaagacaccttcttc | |
| | | cccagcccagaaagttcctgtgatgtcaagctggtcgagaaaagctttgaaaca | |
| | | gatacgaacctaaactttcaaaacctgtcagtgattgggttccgaatcctcctcctg | |
| | | aaagtggccgggtttaatctgctcatgacgctgcggctgtggtccagcGGCTC | |
| | | CGGAGCCACGAACTTCTCTCTGTTAAAGCAAGCAGGA | |
| | | GACGTGGAAGAAAACCCCGGTCCC | |
| | β | GCAGGAGACGTGGAAGAAAACCCCGGTCCCatgggctgca | 20 |
| | | ggctcctctgctgtgtggtcttctgcctcctccaagcaggtcccttggacacagctgt | |
| | | ttcccagactccaaaatacctggtcacacagatgggaaacgacaagtccattaa | |
| | | atgtgaacaaaatctgggccatgatactatgtattggtataaacaggactctaaga | |
| | | aatttctgaagataatgtttagctacaataataaggagctcattataaatgaaaca | |
| | | gttccaaatcgcttctcacctaaatctccagacaaagctcacttaaatcttcacatc | |
| | | aattccctggagcttggtgactctgctgtgtatttctgtgccagcagccaaTGTG | |
| | | CCAGCAGCCAAGCACAGGGGGCGAGTCGGAGCAATC | |
| | | AGCCCCAGCATTTTgaggacctgaacaaggtgttcccacccgaggtcg | |
| | | ctgtgtttgagccatcagaagcagagatctcccacacccaaaaggccacactgg | |
| | | tgtgcctggccacaggcttcttccccgaccacgtggagctgagctggtgggtgaa | |
| | | tgggaaggaggtgcacagtggggtcagcacggacccgcagcccctcaagga | |
| | | gcagcccgccctcaatgactccagatactgcctgagcagccgcctgagggtctc | |
| | | ggccaccttctggcagaacccccgcaaccacttccgctgtcaagtccagttctac | |
| | | gggctctcggagaatgacgagtggacccaggatagggccaaacccgtcaccc | |
| | | agatcgtcagcgccgaggcctggggtagagcagactgtggctttacctcggtgtc | |
| | | ctaccagcaaggggtcctgtctgccaccatcctctatgagatcctgctagggaag | |
| | | gccaccctgtatgctgtgctggtcagcgcccttgtgttgatggccatggtcaagag | |
| | | aaaggatttcTGACTCGAGCGGGATCAATTCCGCCCCCCC | |
| | | CCT | |
| DL | α | CCCTCACTCCTTCTCTAGGCGCCGGAATTCAGATCTAC | 21 |
| | | Catggagaagaatcctttggcagccccattactaatcctctggtttcatcttgactgc | |
| | | gtgagcagcatactgaacgtggaacaaagtcctcagtcactgcatgttcaggag | |
| | | ggagacagcaccaatttcacctgcagcttcccttccagcaatttttatgccttacact | |
| | | ggtacagatgggaaactgcaaaaagccccgaggccttgtttgtaatgactttaaa | |
| | | tggggatgaaaagaagaaaggacgaataagtgccactcttaataccaaggag | |
| | | ggttacagctatttgtacatcaaaggatcccagcctgaagactcagccacatacc | |
| | | tctgtgcctttTGTGCCCGAAACACCGGTAACCAGTTCTATTT | |
| | | TATCCAGAACCCTGACCCTGCCGTGTACCAGCTGAGA | |
| | | GACTCTAAATCCAGTGACAAGTCTGTCTGCCTATTCAC | |
| | | CGATTTTGATTCTCAAACAAATGTGTCACAAAGTAAGGA | |
| | | TTCTGATGTGTATATCACAGACAAAACTGTGCTAGACAT | |
| | | GAGGTCTATGGACTTCAAGAGCAACAGTGCTGTGGCC | |
| | | TGGAGCAACAAATCTGACTTTGCATGTGCAAACGCCTT | |
| | | CAACAACAGCATTATTCCAGAAGACACCTTCTTCCCCA | |
| | | GCCCAGAAAGTTCCTGTGATGTCAAGCTGGTCGAGAAA | |
| | | AGCTTTGAAACAGATACGAACCTAAACTTTCAAAACCTG | |
| | | TCAGTGATTGGGTTCCGAATCCTCCTCCTGAAAGTGGC | |
| | | CGGGTTTAATCTGCTCATGACGCTGCGGCTGTGGTCC | |
| | | AGCGGCTCCGGAGCCACGAACTTCTCTCTGTTAAAGCA | |
| | | AGCAGGAGACGTGGAAGAAAACCCCGGTCCC | |
| | β | GCAGGAGACGTGGAAGAAAACCCCGGTCCCatgagcatcg | 22 |
| | | gcctcctgtgctgtgcagccttgtctctcctgtgggcaggtccagtgaatgctggtgt | |
| | | cactcagaccccaaaattccaggtcctgaagacaggacagagcatgacactgc | |
| | | agtgtgcccaggatatgaaccatgaatacatgtcctggtatcgacaagacccag | |
| | | gcatggggctgaggctgattcattactcagttggtgctggtatcactgaccaagga | |
| | | gaagtccccaatggctacaatgtctccagatcaaccacagaggatttcccgctca | |
| | | ggctgctgtcggctgctccctcccagacatctgtgtacttctgtgccagcagttacT | |
| | | GTGCCAGCAGTTACCAGACAGGGGCTAGCTATGGCTA | |
| | | CACCTTCGAGGACCTGAACAAGGTGTTCCCACCCGAG | |
| | | GTCGCTGTGTTTGAGCCATCAGAAGCAGAGATCTCCCA | |
| | | CACCCAAAAGGCCACACTGGTGTGCCTGGCCACAGGC | |
| | | TTCTTCCCCGACCACGTGGAGCTGAGCTGGTGGGTGA | |
| | | ATGGGAAGGAGGTGCACAGTGGGGTCAGCACGGACC | |
| | | CGCAGCCCCTCAAGGAGCAGCCCGCCCTCAATGACTC | |
| | | CAGATACTGCCTGAGCAGCCGCCTGAGGGTCTCGGCC | |
| | | ACCTTCTGGCAGAACCCCCGCAACCACTTCCGCTGTCA | |
| | | AGTCCAGTTCTACGGGCTCTCGGAGAATGACGAGTGG | |
| | | ACCCAGGATAGGGCCAAACCCGTCACCCAGATCGTCA | |
| | | GCGCCGAGGCCTGGGGTAGAGCAGACTGTGGCTTTAC | |
| | | CTCGGTGTCCTACCAGCAAGGGGTCCTGTCTGCCACC | |
| | | ATCCTCTATGAGATCCTGCTAGGGAAGGCCACCCTGTA | |
| | | TGCTGTGCTGGTCAGCGCCCTTGTGTTGATGGCCATG | |
| | | GTCAAGAGAAAGGATTTCTGACTCGAGCGGGATCAATT | |
| | | CCGCCCCCCCCCT | |

TABLE 1B
Select TCR Clonotypes from ATLAS-seq

| | | | SEQ |
|---|---|---|---|
| TCR | Chain | gBlock Sequences | ID NO |
| A1 | α | CCCTCACTCCTTCTCTAGGCGCCGGAATTCAGATCTACCatgc | 23 |
| | | tcctggagcttatcccactgctggggatacattttgtcctgagaactgccagagcccagtc | |
| | | agtgacccagcctgacatccacatcactgtctctgaaggagcctcactggagttgagatg | |
| | | taactattcctatggggcaacaccttatctcttctggtatgtccagtcccccggccaaggcct | |
| | | ccagctgctcctgaagtacttttcaggagacactctggttcaaggcattaaaggctttgagg | |
| | | ctgaatttaagaggagtcaatcttccttcaatctgaggaaaccctctgtgcattggagtgat | |
| | | gctgctgagtacttctgtgctgtgggtTGTGCTGTGGGTGAGGATTATGGT | |
| | | GGTGCTACAAACAAGCTCATCTTTaatatccagaaccctgaccctgccgt | |
| | | gtaccagctgagagactctaaatccagtgacaagtctgtctgcctattcaccgattttgattc | |
| | | tcaaacaaatgtgtcacaaagtaaggattctgatgtgtatatcacagacaaaactgtgcta | |
| | | gacatgaggtctatggacttcaagagcaacagtgctgtggcctggagcaacaaatctga | |
| | | ctttgcatgtgcaaacgccttcaacaacagcattattccagaagacaccttcttccccagc | |
| | | ccagaaagttcctgtgatgtcaagctggtcgagaaaagctttgaaacagatacgaacct | |
| | | aaactttcaaaacctgtcagtgattgggttccgaatcctcctcctgaaagtggccgggttta | |
| | | atctgctcatgacgctgcggctgtggtccagcGGCTCCGGAGCCACGAACT | |
| | | TCTCTCTGTTAAAGCAAGCAGGAGACGTGGAAGAAAACCCC | |
| | | GGTCCC | |
| | β | GCAGGAGACGTGGAAGAAAACCCCGGTCCCatgggtcctgggcttc | 24 |
| | | tccactggatggccctttgtctccttggaacaggtcatggggatgccatggtcatccagaa | |
| | | cccaagataccaggttacccagtttggaaagccagtgaccctgagttgttctcagactttg | |
| | | aaccataacgtcatgtactggtaccagcagaagtcaagtcaggccccaaagctgctgtt | |
| | | ccactactatgacaaagattttaacaatgaagcagacacccctgataacttccaatccag | |
| | | gaggccgaacacttctttctgctttcttgacatccgctcaccaggcctgggggacacagcc | |
| | | atgtacctgtgtgccaccagcagaTGTGCCACCAGCAGGATCGGACCC | |
| | | CTCCAGGCCTACGAGCAGTACTTCgaggacctgaaaaacgtgttccca | |
| | | cccgaggtcgctgtgtttgagccatcagaagcagagatctcccacacccaaaaggcca | |
| | | cactggtgtgcctggccacaggcttctaccccgaccacgtggagctgagctggtgggtg | |
| | | aatgggaaggaggtgcacagtggggtcagcacagacccgcagcccctcaaggagca | |
| | | gcccgccctcaatgactccagatactgcctgagcagccgcctgagggtctcggccacctt | |
| | | ctggcagaacccccgcaaccacttccgctgtcaagtccagttctacgggctctcggaga | |
| | | atgacgagtggacccaggatagggccaaacctgtcacccagatcgtcagcgccgagg | |
| | | cctggggtagagcagactgtggcttcacctccgagtcttaccagcaaggggtcctgtctg | |
| | | ccaccatcctctatgagatcttgctagggaaggccaccttgtatgccgtgctggtcagtgcc | |
| | | ctcgtgctgatggccatggtcaagagaaaggattccagaggcTGACTCGAGCG | |
| | | GGATCAATTCCGCCCCCCCCCT | |
| A2 | α | CCCTCACTCCTTCTCTAGGCGCCGGAATTCAGATCTACCatgg | 25 |
| | | caggcattcgagctttatttatgtacttgtggctgcagctggactgggtgagcagaggaga | |
| | | gagtgtggggctgcatcttcctaccctgagtgtccaggagggtgacaactctattatcaact | |
| | | gtgcttattcaaacagcgcctcagactacttcatttggtacaagcaagaatctggaaaag | |
| | | gtcctcaattcattatagacattcgttcaaatatggacaaaaggcaaggccaaagagtca | |
| | | ccgttttattgaataagacagtgaaacatctctctctgcaaattgcagctactcaacctgga | |
| | | gactcagctgtctacttttgtgcagagaatTGTGCAGAGAACCAGGCAGGA | |
| | | ACTGCTCTGATCTTTAatatccagaaccctgaccctgccgtgtaccagctgag | |
| | | agactctaaatccagtgacaagtctgtctgcctattcaccgattttgattctcaaacaaatgt | |
| | | gtcacaaagtaaggattctgatgtgtatatcacagacaaaactgtgctagacatgaggtct | |
| | | atggacttcaagagcaacagtgctgtggcctggagcaacaaatctgactttgcatgtgca | |
| | | aacgccttcaacaacagcattattccagaagacaccttcttccccagcccagaaagttcc | |
| | | tgtgatgtcaagctggtcgagaaaagctttgaaacagatacgaacctaaactttcaaaac | |
| | | ctgtcagtgattgggttccgaatcctcctcctgaaagtggccgggtttaatctgctcatgacg | |
| | | ctgcggctgtggtccagcGGCTCCGGAGCCACGAACTTCTCTCTGTT | |
| | | AAAGCAAGCAGGAGACGTGGAAGAAAACCCCGGTCCC | |
| | β | GCAGGAGACGTGGAAGAAAACCCCGGTCCCatgggcccccagctc | 26 |
| | | cttggctatgtggtcctttgccttctaggagcaggccccctggaagcccaagtgacccaga | |
| | | acccaagatacctcatcacagtgactggaaagaagttaacagtgacttgttctcagaata | |
| | | tgaaccatgagtatatgtcctggtatcgacaagacccagggctgggcttaaggcagatct | |
| | | actattcaatgaatgttgaggtgactgataagggagatgttcctgaagggtacaaagtctc | |
| | | tcgaaaagagaagaggaatttccccctgatcctggagtcgcccagccccaaccagacc | |
| | | tctctgtacttctgtgccagcagtttaTGTGCCAGCAGCGCTCAGGGGGTG | |
| | | GTGACTGAAGCTTTCTTTgaggacctgaacaaggtgttcccacccgaggtc | |
| | | gctgtgtttgagccatcagaagcagagatctcccacacccaaaaggccacactggtgtg | |
| | | cctggccacaggcttcttccccgaccacgtggagctgagctggtgggtgaatgggaagg | |
| | | aggtgcacagtggggtcagcacggacccgcagcccctcaaggagcagcccgccctc | |
| | | aatgactccagatactgcctgagcagccgcctgagggtctcggccaccttctggcagaa | |
| | | cccccgcaaccacttccgctgtcaagtccagttctacgggctctcggagaatgacgagtg | |
| | | gacccaggatagggccaaacccgtcacccagatcgtcagcgccgaggcctggggtag | |
| | | agcagactgtggctttacctcggtgtcctaccagcaaggggtcctgtctgccaccatcctct | |
| | | atgagatcctgctagggaaggccaccctgtatgctgtgctggtcagcgcccttgtgttgatg | |
| | | gccatggtcaagagaaaggatttcTGACTCGAGCGGGATCAATTCCGC | |
| | | CCCCCCCCT | |
| A3 | α | CCCTCACTCCTTCTCTAGGCGCCGGAATTCAGATCTACCatgg | 27 |
| | | agaccctcttgggcctgcttatcctttggctgcagctgcaatgggtgagcagcaaacagg | |
| | | aggtgacgcagattcctgcagctctgagtgtcccagaaggagaaaacttggttctcaact | |
| | | gcagtttcactgatagcgctatttacaacctccagtggtttaggcaggaccctgggaaagg | |
| | | tctcacatctctgttgcttattcagtcaagtcagagagagcaaacaagtggaagacttaat | |
| | | gcctcgctggataaatcatcaggacgtagtactttatacattgcagcttctcagcctggtga | |
| | | ctcagccacctacctctgtgctgtgaggTGTGCTGTGAAAGAAGTGGAGG | |
| | | GCGGATCTGAAAAGCTGGTCTTTAatatccagaaccctgaccctgccgt | |
| | | gtaccagctgagagactctaaatccagtgacaagtctgtctgcctattcaccgattttgattc | |
| | | tcaaacaaatgtgtcacaaagtaaggattctgatgtgtatatcacagacaaaactgtgcta | |
| | | gacatgaggtctatggacttcaagagcaacagtgctgtggcctggagcaacaaatctga | |
| | | ctttgcatgtgcaaacgccttcaacaacagcattattccagaagacaccttcttccccagc | |
| | | ccagaaagttcctgtgatgtcaagctggtcgagaaaagctttgaaacagatacgaacct | |
| | | aaactttcaaaacctgtcagtgattgggttccgaatcctcctcctgaaagtggccgggttta | |
| | | atctgctcatgacgctgcggctgtggtccagcGGCTCCGGAGCCACGAACT | |
| | | TCTCTCTGTTAAAGCAAGCAGGAGACGTGGAAGAAAACCCC | |
| | | GGTCCC | |
| | β | GCAGGAGACGTGGAAGAAAACCCCGGTCCCatgggcccccagctc | 28 |
| | | cttggctatgtggtcctttgccttctaggagcaggccccctggaagcccaagtgacccaga | |
| | | acccaagatacctcatcacagtgactggaaagaagttaacagtgacttgttctcagaata | |
| | | tgaaccatgagtatatgtcctggtatcgacaagacccagggctgggcttaaggcagatct | |
| | | actattcaatgaatgttgaggtgactgataagggagatgttcctgaagggtacaaagtctc | |
| | | tcgaaaagagaagaggaatttccccctgatcctggagtcgcccagccccaaccagacc | |
| | | tctctgtacttctgtgccagcagtttaTGTGCCAGCAGTGTGACAGGGTTC | |
| | | TGGGAGCAGTACTTCgaggacctgaaaaacgtgttcccacccgaggtcgctg | |
| | | tgtttgagccatcagaagcagagatctcccacacccaaaaggccacactggtgtgcctg | |
| | | gccacaggcttctaccccgaccacgtggagctgagctggtgggtgaatgggaaggagg | |
| | | tgcacagtggggtcagcacagacccgcagcccctcaaggagcagcccgccctcaatg | |
| | | actccagatactgcctgagcagccgcctgagggtctcggccaccttctggcagaacccc | |
| | | cgcaaccacttccgctgtcaagtccagttctacgggctctcggagaatgacgagtggacc | |
| | | caggatagggccaaacctgtcacccagatcgtcagcgccgaggcctggggtagagca | |
| | | gactgtggcttcacctccgagtcttaccagcaaggggtcctgtctgccaccatcctctatga | |
| | | gatcttgctagggaaggccaccttgtatgccgtgctggtcagtgccctcgtgctgatggcc | |
| | | atggtcaagagaaaggattccagaggcTGACTCGAGCGGGATCAATTC | |
| | | CGCCCCCCCCCT | |
| A4 | α | CCCTCACTCCTTCTCTAGGCGCCGGAATTCAGATCTACCatgc | 29 |
| | | tcctgctgctcgtcccagcgttccaggtgatttttaccctgggaggaaccagagcccagtct | |
| | | gtgacccagcttgacagccaagtccctgtctttgaagaagcccctgtggagctgaggtgc | |
| | | aactactcatcgtctgtttcagtgtatctcttctggtatgtgcaataccccaaccaaggactcc | |
| | | agcttctcctgaagtatttatcaggatccaccctggttgaaagcatcaacggttttgaggctg | |
| | | aatttaacaagagtcaaacttccttccacttgaggaaaccctcagtccatataagcgaca | |
| | | cggctgagtacttctgtgctgtgagtTGTGCTGTGAGTGATCAAGGGGAC | |
| | | TACAAGCTCAGCTTTaatatccagaaccctgaccctgccgtgtaccagctgag | |
| | | agactctaaatccagtgacaagtctgtctgcctattcaccgattttgattctcaaacaaatgt | |
| | | gtcacaaagtaaggattctgatgtgtatatcacagacaaaactgtgctagacatgaggtct | |
| | | atggacttcaagagcaacagtgctgtggcctggagcaacaaatctgactttgcatgtgca | |
| | | aacgccttcaacaacagcattattccagaagacaccttcttccccagcccagaaagttcc | |
| | | tgtgatgtcaagctggtcgagaaaagctttgaaacagatacgaacctaaactttcaaaac | |
| | | ctgtcagtgattgggttccgaatcctcctcctgaaagtggccgggtttaatctgctcatgacg | |
| | | ctgcggctgtggtccagcGGCTCCGGAGCCACGAACTTCTCTCTGTT | |
| | | AAAGCAAGCAGGAGACGTGGAAGAAAACCCCGGTCCC | |
| | β | GCAGGAGACGTGGAAGAAAACCCCGGTCCCatggacaccagagt | 30 |
| | | actctgctgtgcggtcatctgtcttctgggggcaggtctctcaaatgccggcgtcatgcaga | |
| | | acccaagacacctggtcaggaggaggggacaggaggcaagactgagatgcagccc | |
| | | aatgaaaggacacagtcatgtttactggtatcggcagctcccagaggaaggtctgaaatt | |
| | | catggtttatctccagaaagaaaatatcatagatgagtcaggaatgccaaaggaacgatt | |
| | | ttctgctgaatttcccaaagagggccccagcatcctgaggatccagcaggtagtgcgag | |
| | | gagattcggcagcttatttctgtgccagctcaccaTGTGCCAGCTCACCTCGG | |
| | | GGGGGCCGGATCCACGAGCAGTACTTCgaggacctgaaaaacgtgt | |
| | | tcccacccgaggtcgctgtgtttgagccatcagaagcagagatctcccacacccaaaag | |
| | | gccacactggtgtgcctggccacaggcttctaccccgaccacgtggagctgagctggtg | |
| | | ggtgaatgggaaggaggtgcacagtggggtcagcacagacccgcagcccctcaagg | |
| | | agcagcccgccctcaatgactccagatactgcctgagcagccgcctgagggtctcggc | |
| | | caccttctggcagaacccccgcaaccacttccgctgtcaagtccagttctacgggctctcg | |
| | | gagaatgacgagtggacccaggatagggccaaacctgtcacccagatcgtcagcgcc | |
| | | gaggcctggggtagagcagactgtggcttcacctccgagtcttaccagcaaggggtcct | |
| | | gtctgccaccatcctctatgagatcttgctagggaaggccaccttgtatgccgtgctggtca | |
| | | gtgccctcgtgctgatggccatggtcaagagaaaggattccagaggcTGACTCGA | |
| | | GCGGGATCAATTCCGCCCCCCCCCT | |
| A5 | α | CCCTCACTCCTTCTCTAGGCGCCGGAATTCAGATCTACCatgg | 31 |
| | | agaccctcttgggcctgcttatcctttggctgcagctgcaatgggtgagcagcaaacagg | |
| | | aggtgacgcagattcctgcagctctgagtgtcccagaaggagaaaacttggttctcaact | |
| | | gcagtttcactgatagcgctatttacaacctccagtggtttaggcaggaccctgggaaagg | |
| | | tctcacatctctgttgcttattcagtcaagtcagagagagcaaacaagtggaagacttaat | |
| | | gcctcgctggataaatcatcaggacgtagtactttatacattgcagcttctcagcctggtga | |
| | | ctcagccacctacctctgtgctgtgaggTGTGCTGTGAGTTTTGGCTCTAG | |
| | | CAACACAGGCAAACTAATCTTTAatatccagaaccctgaccctgccgtgta | |
| | | ccagctgagagactctaaatccagtgacaagtctgtctgcctattcaccgattttgattctca | |
| | | aacaaatgtgtcacaaagtaaggattctgatgtgtatatcacagacaaaactgtgctaga | |
| | | catgaggtctatggacttcaagagcaacagtgctgtggcctggagcaacaaatctgactt | |
| | | tgcatgtgcaaacgccttcaacaacagcattattccagaagacaccttcttccccagccc | |
| | | agaaagttcctgtgatgtcaagctggtcgagaaaagctttgaaacagatacgaacctaa | |
| | | actttcaaaacctgtcagtgattgggttccgaatcctcctcctgaaagtggccgggtttaatc | |
| | | tgctcatgacgctgcggctgtggtccagcGGCTCCGGAGCCACGAACTTC | |
| | | TCTCTGTTAAAGCAAGCAGGAGACGTGGAAGAAAACCCCGG | |
| | | TCCC | |
| | β | GCAGGAGACGTGGAAGAAAACCCCGGTCCCatgggctgcaggctg | 32 |
| | | ctctgctgtgcggttctctgtctcctgggagcgggtgagttggtccccatggaaacgggagt | |
| | | tacgcagacaccaagacacctggtcatgggaatgacaaataagaagtctttgaaatgtg | |
| | | aacaacatctgggtcataacgctatgtattggtacaagcaaagtgctaagaagccactg | |
| | | gagctcatgtttgtctacagtcttgaagaacgggttgaaaacaacagtgtgccaagtcgct | |
| | | tctcacctgaatgccccaacagctctcacttattccttcacctacacaccctgcagccaga | |
| | | agactcggccctgtatctctgcgccagcagccaaTGCGCCAGCAGCCAAGA | |
| | | TAATTCTAGGGCAGCCTACAATGAGCAGTTCTTCgaggacctga | |
| | | aaaacgtgttcccacccgaggtcgctgtgtttgagccatcagaagcagagatctcccac | |
| | | acccaaaaggccacactggtgtgcctggccacaggcttctaccccgaccacgtggagc | |
| | | tgagctggtgggtgaatgggaaggaggtgcacagtggggtcagcacagacccgcagc | |
| | | ccctcaaggagcagcccgccctcaatgactccagatactgcctgagcagccgcctgag | |
| | | ggtctcggccaccttctggcagaacccccgcaaccacttccgctgtcaagtccagttctac | |
| | | gggctctcggagaatgacgagtggacccaggatagggccaaacctgtcacccagatc | |
| | | gtcagcgccgaggcctggggtagagcagactgtggcttcacctccgagtcttaccagca | |
| | | aggggtcctgtctgccaccatcctctatgagatcttgctagggaaggccaccttgtatgcc | |
| | | gtgctggtcagtgccctcgtgctgatggccatggtcaagagaaaggattccagaggcT | |
| | | GACTCGAGCGGGATCAATTCCGCCCCCCCCCT | |
| A14 | α | CCCTCACTCCTTCTCTAGGCGCCGGAATTCAGATCTACCatgg | 33 |
| | | caggcattcgagctttatttatgtacttgtggctgcagctggactgggtgagcagaggaga | |
| | | gagtgtggggctgcatcttcctaccctgagtgtccaggagggtgacaactctattatcaact | |
| | | gtgcttattcaaacagcgcctcagactacttcatttggtacaagcaagaatctggaaaag | |
| | | gtcctcaattcattatagacattcgttcaaatatggacaaaaggcaaggccaaagagtca | |
| | | ccgttttattgaataagacagtgaaacatctctctctgcaaattgcagctactcaacctgga | |
| | | gactcagctgtctacttttgtgcagagaatTGTGCAGAGTCCTCTTCCGGGT | |
| | | ATGCACTCAACTTCaatatccagaaccctgaccctgccgtgtaccagctgaga | |
| | | gactctaaatccagtgacaagtctgtctgcctattcaccgattttgattctcaaacaaatgtgt | |
| | | cacaaagtaaggattctgatgtgtatatcacagacaaaactgtgctagacatgaggtctat | |
| | | ggacttcaagagcaacagtgctgtggcctggagcaacaaatctgactttgcatgtgcaa | |
| | | acgccttcaacaacagcattattccagaagacaccttcttccccagcccagaaagttcct | |
| | | gtgatgtcaagctggtcgagaaaagctttgaaacagatacgaacctaaactttcaaaac | |
| | | ctgtcagtgattgggttccgaatcctcctcctgaaagtggccgggtttaatctgctcatgacg | |
| | | ctgcggctgtggtccagcGGCTCCGGAGCCACGAACTTCTCTCTGTT | |
| | | AAAGCAAGCAGGAGACGTGGAAGAAAACCCCGGTCCC | |
| | β | GCAGGAGACGTGGAAGAAAACCCCGGTCCCatgggcccccagctc | 34 |
| | | cttggctatgtggtcctttgccttctaggagcaggccccctggaagcccaagtgacccaga | |
| | | acccaagatacctcatcacagtgactggaaagaagttaacagtgacttgttctcagaata | |
| | | tgaaccatgagtatatgtcctggtatcgacaagacccagggctgggcttaaggcagatct | |
| | | actattcaatgaatgttgaggtgactgataagggagatgttcctgaagggtacaaagtctc | |
| | | tcgaaaagagaagaggaatttccccctgatcctggagtcgcccagccccaaccagacc | |
| | | tctctgtacttctgtgccagcagtttaTGTGCCAGCAGTTCACAGGGGGCT | |
| | | CGAACTGAAGCTTTCTTTgaggacctgaacaaggtgttcccacccgaggtc | |
| | | gctgtgtttgagccatcagaagcagagatctcccacacccaaaaggccacactggtgtg | |
| | | cctggccacaggcttcttccccgaccacgtggagctgagctggtgggtgaatgggaagg | |
| | | aggtgcacagtggggtcagcacggacccgcagcccctcaaggagcagcccgccctc | |
| | | aatgactccagatactgcctgagcagccgcctgagggtctcggccaccttctggcagaa | |
| | | cccccgcaaccacttccgctgtcaagtccagttctacgggctctcggagaatgacgagtg | |
| | | gacccaggatagggccaaacccgtcacccagatcgtcagcgccgaggcctggggtag | |
| | | agcagactgtggctttacctcggtgtcctaccagcaaggggtcctgtctgccaccatcctct | |
| | | atgagatcctgctagggaaggccaccctgtatgctgtgctggtcagcgcccttgtgttgatg | |
| | | gccatggtcaagagaaaggatttcTGACTCGAGCGGGATCAATTCCGC | |
| | | CCCCCCCCT | |
| A41 | α | CCCTCACTCCTTCTCTAGGCGCCGGAATTCAGATCTACCatgg | 35 |
| | | catgccctggcttcctgtgggcacttgtgatctccacctgtcttgaatttagcatggctcagac | |
| | | agtcactcagtctcaaccagagatgtctgtgcaggaggcagagaccgtgaccctgagct | |
| | | gcacatatgacaccagtgagagtgattattatttattctggtacaagcagcctcccagcag | |
| | | gcagatgattctcgttattcgccaagaagcttataagcaacagaatgcaacagagaatc | |
| | | gtttctctgtgaacttccagaaagcagccaaatccttcagtctcaagatctcagactcaca | |
| | | gctgggggatgccgcgatgtatttctgtgcttataggagcTGTGCTGCTGAGGT | |
| | | CAATTCCGGGTATGCACTCAACTTCaatatccagaaccctgaccctgcc | |
| | | gtgtaccagctgagagactctaaatccagtgacaagtctgtctgcctattcaccgattttgat | |
| | | tctcaaacaaatgtgtcacaaagtaaggattctgatgtgtatatcacagacaaaactgtgc | |
| | | tagacatgaggtctatggacttcaagagcaacagtgctgtggcctggagcaacaaatct | |
| | | gactttgcatgtgcaaacgccttcaacaacagcattattccagaagacaccttcttcccca | |
| | | gcccagaaagttcctgtgatgtcaagctggtcgagaaaagctttgaaacagatacgaac | |
| | | ctaaactttcaaaacctgtcagtgattgggttccgaatcctcctcctgaaagtggccgggttt | |
| | | aatctgctcatgacgctgcggctgtggtccagcGGCTCCGGAGCCACGAAC | |
| | | TTCTCTCTGTTAAAGCAAGCAGGAGACGTGGAAGAAAACCC | |
| | | CGGTCCC | |
| | β | GCAGGAGACGTGGAAGAAAACCCCGGTCCCatgggcaccaggct | 36 |
| | | cctcttctgggtggccttctgtctcctgggggcagatcacacaggagctggagtctcccagt | |
| | | cccccagtaacaaggtcacagagaagggaaaggatgtagagctcaggtgtgatccaa | |
| | | tttcaggtcatactgccctttactggtaccgacagagcctggggcagggcctggagttttta | |
| | | atttacttccaaggcaacagtgcaccagacaaatcagggctgcccagtgatcgcttctct | |
| | | gcagagaggactgggggatccgtctccactctgacgatccagcgcacacagcaggag | |
| | | gactcggccgtgtatctctgtgccagcagcttaTGTGCCAGCAGCGTACGAG | |
| | | AGACCCAGTACTTCgaggacctgaaaaacgtgttcccacccgaggtcgctgtg | |
| | | tttgagccatcagaagcagagatctcccacacccaaaaggccacactggtgtgcctggc | |
| | | cacaggcttctaccccgaccacgtggagctgagctggtgggtgaatgggaaggaggtg | |
| | | cacagtggggtcagcacagacccgcagcccctcaaggagcagcccgccctcaatga | |
| | | ctccagatactgcctgagcagccgcctgagggtctcggccaccttctggcagaaccccc | |
| | | gcaaccacttccgctgtcaagtccagttctacgggctctcggagaatgacgagtggaccc | |
| | | aggatagggccaaacctgtcacccagatcgtcagcgccgaggcctggggtagagcag | |
| | | actgtggcttcacctccgagtcttaccagcaaggggtcctgtctgccaccatcctctatgag | |
| | | atcttgctagggaaggccaccttgtatgccgtgctggtcagtgccctcgtgctgatggccat | |
| | | ggtcaagagaaaggattccagaggcTGACTCGAGCGGGATCAATTCC | |
| | | GCCCCCCCCCT | |
| A42 | α | CCCTCACTCCTTCTCTAGGCGCCGGAATTCAGATCTACCatga | 37 |
| | | agaggatattgggagctctgctggggctcttgagtgcccaggtttgctgtgtgagaggaat | |
| | | acaagtggagcagagtcctccagacctgattctccaggagggagccaattccacgctg | |
| | | cggtgcaatttttctgactctgtgaacaatttgcagtggtttcatcaaaacccttggggacag | |
| | | ctcatcaacctgttttacattccctcagggacaaaacagaatggaagattaagcgccacg | |
| | | actgtcgctacggaacgctacagcttattgtacatttcctcttcccagaccacagactcagg | |
| | | cgtttatttctgtgctgtggagTGTGCTGGGGGAGGTGCTGACGGACTC | |
| | | ACCTTTaatatccagaaccctgaccctgccgtgtaccagctgagagactctaaatcc | |
| | | agtgacaagtctgtctgcctattcaccgattttgattctcaaacaaatgtgtcacaaagtaa | |
| | | ggattctgatgtgtatatcacagacaaaactgtgctagacatgaggtctatggacttcaag | |
| | | agcaacagtgctgtggcctggagcaacaaatctgactttgcatgtgcaaacgccttcaac | |
| | | aacagcattattccagaagacaccttcttccccagcccagaaagttcctgtgatgtcaagc | |
| | | tggtcgagaaaagctttgaaacagatacgaacctaaactttcaaaacctgtcagtgattg | |
| | | ggttccgaatcctcctcctgaaagtggccgggtttaatctgctcatgacgctgcggctgtgg | |
| | | tccagcGGCTCCGGAGCCACGAACTTCTCTCTGTTAAAGCAAG | |
| | | CAGGAGACGTGGAAGAAAACCCCGGTCCC | |
| | β | GCAGGAGACGTGGAAGAAAACCCCGGTCCCatgctgagtcttctgct | 38 |
| | | ccttctcctgggactaggctctgtgttcagtgctgtcatctctcaaaagccaagcagggata | |
| | | tctgtcaacgtggaacctccctgacgatccagtgtcaagtcgatagccaagtcaccatga | |
| | | tgttctggtaccgtcagcaacctggacagagcctgacactgatcgcaactgcaaatcag | |
| | | ggctctgaggccacatatgagagtggatttgtcattgacaagtttcccatcagccgcccaa | |
| | | acctaacattctcaactctgactgtgagcaacatgagccctgaagacagcagcatatatc | |
| | | tctgcagcgttgaaTGCAGCGTTGTACCCCGGGACAGGGTAGCTG | |
| | | GAAACACCATATATTTTgaggacctgaacaaggtgttcccacccgaggtcgc | |
| | | tgtgtttgagccatcagaagcagagatctcccacacccaaaaggccacactggtgtgcc | |
| | | tggccacaggcttcttccccgaccacgtggagctgagctggtgggtgaatgggaaggag | |
| | | gtgcacagtggggtcagcacggacccgcagcccctcaaggagcagcccgccctcaat | |
| | | gactccagatactgcctgagcagccgcctgagggtctcggccaccttctggcagaaccc | |
| | | ccgcaaccacttccgctgtcaagtccagttctacgggctctcggagaatgacgagtggac | |
| | | ccaggatagggccaaacccgtcacccagatcgtcagcgccgaggcctggggtagagc | |
| | | agactgtggctttacctcggtgtcctaccagcaaggggtcctgtctgccaccatcctctatg | |
| | | agatcctgctagggaaggccaccctgtatgctgtgctggtcagcgcccttgtgttgatggc | |
| | | catggtcaagagaaaggatttcTGACTCGAGCGGGATCAATTCCGCC | |
| | | CCCCCCCT | |

TABLE 2A
TCRα Sequences

| | | TCRα CDR3 | SEQ | TCRα CDR3 | SEQ | | | |
|---|---|---|---|---|---|---|---|---|
| Methods | TCR | Amino Acids | ID NO | Nucleotides | ID NO | TRAV | TRAJ | TRAC |
| Dextramer | D1 | CAASNTYGN | 39 | TGTGCAGC | 58 | TRAV13- | TRAJ7 | TRAC |
| Staining | | NRLAF | | AAGTAATAC | | 1 | | |
| | | | | CTATGGGAA | | | | |
| | | | | CAACAGACT | | | | |
| | | | | CGCTTTT | | | | |
| | D2 | CAVHWMDSS | 40 | TGTGCTGTC | 59 | TRAV1- | TRAJ12 | TRAC |
| | | YKLIF | | CATTGGATG | | 2 | | |
| | | | | GATAGCAG | | | | |
| | | | | CTATAAATT | | | | |
| | | | | GATCTTC | | | | |
| | D3 | CAYRPSYSG | 41 | TGTGCTTAT | 60 | TRAV38- | TRAJ29 | TRAC |
| | | NTPLVF | | AGGCCCTC | | 2/DV8 | | |
| | | | | TTATTCAGG | | | | |
| | | | | AAACACACC | | | | |
| | | | | TCTTGTCTT | | | | |
| | | | | T | | | | |
| | D4 | CASQGTNFG | 42 | TGTGCCTCC | 61 | TRAV24 | TRAJ48 | TRAC |
| | | NEKLTF | | CAGGGCAC | | | | |
| | | | | TAACTTTGG | | | | |
| | | | | AAATGAGAA | | | | |
| | | | | ATTAACCTT | | | | |
| | | | | T | | | | |
| | D5 | CAVRDENSG | 43 | TGTGCTGTG | 62 | TRAV3 | TRAJ11 | TRAC |
| | | YSTLTF | | AGAGACGA | | | | |
| | | | | AAATTCAGG | | | | |
| | | | | ATACAGCAC | | | | |
| | | | | CCTCACCTT | | | | |
| | | | | T | | | | |
| | D10 | CAVFPSNDY | 44 | TGTGCTGTG | 63 | TRAV3 | TRAJ20 | TRAC |
| | | KLSF | | TTCCCCTCT | | | | |
| | | | | AACGACTAC | | | | |
| | | | | AAGCTCAG | | | | |
| | | | | CTTT | | | | |
| | D47 | CAYVRDDKIIF | 45 | TGTGCCTAC | 64 | TRAV17 | TRAJ30 | TRAC |
| | | | | GTCAGAGAT | | | | |
| | | | | GACAAGATC | | | | |
| | | | | ATCTTT | | | | |
| | D67 | CAFPYNNND | 46 | TGTGCCTTC | 65 | TRAV24 | TRAJ43 | TRAC |
| | | MRF | | CCTTACAAT | | | | |
| | | | | AACAATGAC | | | | |
| | | | | ATGCGCTTT | | | | |
| | D77 | CALNRDDKIIF | 47 | TGTGCCCT | 66 | TRAV17 | TRAJ30 | TRAC |
| | | | | GAACAGAG | | | | |
| | | | | ATGACAAGA | | | | |
| | | | | TCATCTTT | | | | |
| | D79 | CAVSVTGRQ | 48 | TGTGCTGTG | 67 | TRAV8- | TRAJ23 | TRAC |
| | | GGKLIF | | AGTGTCAC | | 4 | | |
| | | | | GGGAAGGC | | | | |
| | | | | AGGGAGGA | | | | |
| | | | | AAGCTTATC | | | | |
| | | | | TTC | | | | |
| | DL | CARNTGNQF | 49 | TGTGCCCG | 68 | TRAV24 | TRAJ49 | TRAC |
| | | YF | | AAACACCG | | | | |
| | | | | GTAACCAGT | | | | |
| | | | | TCTATTTT | | | | |
| ATLAS- | A1 | CAVGEDYGG | 50 | TGTGCTGTG | 69 | TRAV8- | TRAJ32 | TRAC |
| seq | | ATNKLIF | | GGTGAGGA | | 3 | | |
| | | | | TTATGGTGG | | | | |
| | | | | TGCTACAAA | | | | |
| | | | | CAAGCTCAT | | | | |
| | | | | CTTT | | | | |
| | A2 | CAENQAGTA | 51 | TGTGCAGA | 70 | TRAV13- | TRAJ15 | TRAC |
| | | LIF | | GAACCAGG | | 2 | | |
| | | | | CAGGAACT | | | | |
| | | | | GCTCTGATC | | | | |
| | | | | TTT | | | | |
| | A3 | CAVKEVEGG | 52 | TGTGCTGTG | 71 | TRAV21 | TRAJ57 | TRAC |
| | | SEKLVF | | AAAGAAGTG | | | | |
| | | | | GAGGGCGG | | | | |
| | | | | ATCTGAAAA | | | | |
| | | | | GCTGGTCTT | | | | |
| | | | | T | | | | |
| | A4 | CAVSDQGDY | 53 | TGTGCTGTG | 72 | TRAV8- | TRAJ20 | TRAC |
| | | KLSF | | AGTGATCAA | | 6 | | |
| | | | | GGGGACTA | | | | |
| | | | | CAAGCTCA | | | | |
| | | | | GCTTT | | | | |
| | A5 | CAVSFGSSN | 54 | TGTGCTGTG | 73 | TRAV21 | TRAJ37 | TRAC |
| | | TGKLIF | | AGTTTTGGC | | | | |
| | | | | TCTAGCAAC | | | | |
| | | | | ACAGGCAA | | | | |
| | | | | ACTAATCTT | | | | |
| | | | | T | | | | |
| | A14 | CAESSSGYA | 55 | TGTGCAGA | 74 | TRAV13- | TRAJ41 | TRAC |
| | | LNF | | GTCCTCTTC | | 2 | | |
| | | | | CGGGTATG | | | | |
| | | | | CACTCAACT | | | | |
| | | | | TC | | | | |
| | A41 | CAAEVNSGY | 56 | TGTGCTGCT | 75 | TRAV38- | TRAJ41 | TRAC |
| | | ALNF | | GAGGTCAAT | | 2/DV8 | | |
| | | | | TCCGGGTAT | | | | |
| | | | | GCACTCAAC | | | | |
| | | | | TTC | | | | |
| | A42 | CAGGGADGL | 57 | TGTGCTGG | 76 | TRAV22 | TRAJ45 | TRAC |
| | | TF | | GGGAGGTG | | | | |
| | | | | CTGACGGA | | | | |
| | | | | CTCACCTTT | | | | |

TABLE 2B
TCRβ Sequences

| | | TCRβ CDR3 | SEQ | TCRβ CDR3 | SEQ | | | |
|---|---|---|---|---|---|---|---|---|
| Methods | TCR | Amino Acids | ID NO | Nucleotides | ID NO | TRBV | TRBJ | TRBC |
| Dextramer | D1 | CASSLKRGLT | 77 | TGCGCCAG | 96 | TRBV5- | TRBJ2- | TRBC2 |
| Staining | | DTQYF | | CAGCCTAAA | | 1 | 3 | |
| | | | | ACGGGGGC | | | | |
| | | | | TGACAGATA | | | | |
| | | | | CGCAGTATT | | | | |
| | | | | TT | | | | |
| | D2 | CATSDRQGY | 78 | TGTGCCAC | 97 | TRBV24- | TRBJ2- | TRBC2 |
| | | NEQFF | | CAGTGACC | | 1 | 1 | |
| | | | | GGCAGGGG | | | | |
| | | | | TACAATGAG | | | | |
| | | | | CAGTTCTTC | | | | |
| | D3 | CASSLRTNTY | 79 | TGCGCCAG | 98 | TRBV4- | TRBJ2- | TRBC2 |
| | | EQYF | | CAGCCTAC | | 1 | 7 | |
| | | | | GGACCAATA | | | | |
| | | | | CCTACGAG | | | | |
| | | | | CAGTACTTC | | | | |
| | D4 | CASSLYGGN | 80 | TGTGCCAG | 99 | TRBV27 | TRBJ1- | TRBC1 |
| | | TEAFF | | CAGTTTATA | | | 1 | |
| | | | | TGGGGGAA | | | | |
| | | | | ACACTGAAG | | | | |
| | | | | CTTTCTTT | | | | |
| | D5 | CASSLKDSY | 81 | TGTGCCAG | 100 | TRBV11- | TRBJ1- | TRBC1 |
| | | NEKLFF | | CAGCTTAAA | | 2 | 4 | |
| | | | | GGACAGCT | | | | |
| | | | | ATAATGAAA | | | | |
| | | | | AACTGTTTT | | | | |
| | | | | TT | | | | |
| | D10 | CATSDPGRA | 82 | TGTGCCAC | 101 | TRBV24- | TRBJ1- | TRBC1 |
| | | YNQPQHF | | CAGTGATCC | | 1 | 5 | |
| | | | | GGGGAGGG | | | | |
| | | | | CTTACAATC | | | | |
| | | | | AGCCCCAG | | | | |
| | | | | CATTTT | | | | |
| | D47 | CASSLASGG | 83 | TGTGCCAG | 102 | TRBV12- | TRBJ1- | TRBC1 |
| | | NSPLHF | | CAGTTTAGC | | 3 | 6 | |
| | | | | GTCAGGGG | | | | |
| | | | | GGAATTCAC | | | | |
| | | | | CCCTCCACT | | | | |
| | | | | TT | | | | |
| | D67 | CASSLEGYTE | 84 | TGTGCCAG | 103 | TRBV27 | TRBJ1- | TRBC1 |
| | | AFF | | CAGTTTAGA | | | 1 | |
| | | | | GGGTTACA | | | | |
| | | | | CTGAAGCTT | | | | |
| | | | | TCTTT | | | | |
| | D77 | CASSLAGTG | 85 | TGTGCCAG | 104 | TRBV7- | TRBJ2- | TRBC2 |
| | | PTGELFF | | CAGCTTAGC | | 6 | 2 | |
| | | | | CGGGACAG | | | | |
| | | | | GGCCGACC | | | | |
| | | | | GGGGAGCT | | | | |
| | | | | GTTTTTT | | | | |
| | D79 | CASSQAQGA | 86 | TGTGCCAG | 105 | TRBV3- | TRBJ1- | TRBC1 |
| | | SRSNQPQHF | | CAGCCAAG | | 1 | 5 | |
| | | | | CACAGGGG | | | | |
| | | | | GCGAGTCG | | | | |
| | | | | GAGCAATCA | | | | |
| | | | | GCCCCAGC | | | | |
| | | | | ATTTT | | | | |
| | DL | CASSYQTGA | 87 | TGTGCCAG | 106 | TRBV6- | TRBJ1- | TRBC1 |
| | | SYGYTF | | CAGTTACCA | | 5 | 2 | |
| | | | | GACAGGGG | | | | |
| | | | | CTAGCTATG | | | | |
| | | | | GCTACACCT | | | | |
| | | | | TC | | | | |
| ATLAS- | A1 | CATSRIGPLQ | 88 | TGTGCCAC | 107 | TRBV15 | TRBJ2- | TRBC2 |
| seq | | AYEQYF | | CAGCAGGA | | | 7 | |
| | | | | TCGGACCC | | | | |
| | | | | CTCCAGGC | | | | |
| | | | | CTACGAGC | | | | |
| | | | | AGTACTTC | | | | |
| | A2 | CASSAQGVV | 89 | TGTGCCAG | 108 | TRBV27 | TRBJ1- | TRBC1 |
| | | TEAFF | | CAGCGCTC | | | 1 | |
| | | | | AGGGGGTG | | | | |
| | | | | GTGACTGAA | | | | |
| | | | | GCTTTCTTT | | | | |
| | A3 | CASSVTGFW | 90 | TGTGCCAG | 109 | TRBV27 | TRBJ2- | TRBC2 |
| | | EQYF | | CAGTGTGA | | | 7 | |
| | | | | CAGGGTTCT | | | | |
| | | | | GGGAGCAG | | | | |
| | | | | TACTTC | | | | |
| | A4 | CASSPRGGRI | 91 | TGTGCCAG | 110 | TRBV18 | TRBJ2- | TRBC2 |
| | | HEQYF | | CTCACCTCG | | | 7 | |
| | | | | GGGGGGCC | | | | |
| | | | | GGATCCAC | | | | |
| | | | | GAGCAGTA | | | | |
| | | | | CTTC | | | | |
| | A5 | CASSQDNSR | 92 | TGCGCCAG | 111 | TRBV4- | TRBJ2- | TRBC2 |
| | | AAYNEQFF | | CAGCCAAG | | 3 | 1 | |
| | | | | ATAATTCTA | | | | |
| | | | | GGGCAGCC | | | | |
| | | | | TACAATGAG | | | | |
| | | | | CAGTTCTTC | | | | |
| | A14 | CASSSQGAR | 93 | TGTGCCAG | 112 | TRBV27 | TRBJ1- | TRBC1 |
| | | TEAFF | | CAGTTCACA | | | 1 | |
| | | | | GGGGGCTC | | | | |
| | | | | GAACTGAA | | | | |
| | | | | GCTTTCTTT | | | | |
| | A41 | CASSVRETQ | 94 | TGTGCCAG | 113 | TRBV7- | TRBJ2- | TRBC2 |
| | | YF | | CAGCGTAC | | 2 | 5 | |
| | | | | GAGAGACC | | | | |
| | | | | CAGTACTTC | | | | |
| | A42 | CSVVPRDRV | 95 | TGCAGCGTT | 114 | TRBV29- | TRBJ1- | TRBC1 |
| | | AGNTIYF | | GTACCCCG | | 1 | 3 | |
| | | | | GGACAGGG | | | | |
| | | | | TAGCTGGAA | | | | |
| | | | | ACACCATAT | | | | |
| | | | | ATTTT | | | | |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. References

The following references, to the extent that they provide exemplary procedural or other details supplementary to those set forth herein, are specifically incorporated herein by reference.
Altan-Bonnet, G. and R. Mukherjee (2019). “Cytokine-mediated communication: a quantitative appraisal of immune complexity.” Nat Rev Immunol 19 (4): 205-217.
Ashburner, M., C. A. Ball, J. A. Blake, D. Botstein, H. Butler, J. M. Cherry, A. P. Davis, K. Dolinski. S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin and G. Sherlock (2000). “Gene ontology: tool for the unification of biology. The Gene Ontology Consortium.” Nat Genet 25 (1): 25-29.
Bentzen, A. K., A. M. Marquard, R. Lyngaa, S. K. Saini, S. Ramskov, M. Donia, L. Such, A. J. Furness, N. McGranahan, R. Rosenthal, P. T. Straten, Z. Szallasi, I. M. Svane, C. Swanton, S. A. Quezada, S. N. Jakobsen, A. C. Eklund and S. R. Hadrup (2016). “Large-scale detection of antigen-specific T cells using peptide-MHC-I multimers labeled with DNA barcodes.” Nat Biotechnol 34 (10): 1037-1045.
Bibby, J. A., D. Agarwal, T. Freiwald, N. Kunz, N. S. Merle, E. E. West, P. Singh, A. Larochelle, F. Chinian, S. Mukherjee, B. Afzali, C. Kemper and N. R. Zhang (2022). “Systematic single-cell pathway analysis to characterize early T cell activation.” Cell Rep 41 (8): 111697.
Borbulevych, O. Y., S. M. Santhanagopolan, M. Hossain and B. M. Baker (2011). “TCRs used in cancer gene therapy cross-react with MART-1/Melan-A tumor antigens via distinct mechanisms.” J Immunol 187 (5): 2453-2463.
Boshtam, M., S. Asgary, S. Kouhpayeh, L. Shariati and H. Khanahmad (2017). “Aptamers Against Pro- and Anti-Inflammatory Cytokines: A Review.” Inflammation 40 (1): 340-349.
Butler, A., P. Hoffman, P. Smibert, E. Papalexi and R. Satija (2018). “Integrating single-cell transcriptomic data across different conditions, technologies, and species.” Nat Biotechnol 36 (5): 411-420.
Campbell, J. D., A. Foerster, V. Lasmanowicz, M. Niemoller, A. Scheffold, M. Fahrendorff, G. Rauser, M. Assenmacher and A. Richter (2011). “Rapid detection, enrichment and propagation of specific T cell subsets based on cytokine secretion.” Clin Exp Immunol 163 (1): 1-10.
Chang, J. (2021). “MHC multimer: A Molecular Toolbox for Immunologists.” Mol Cells 44 (5): 328-334.
Chen, G., X. Yang, A. Ko, X. Sun, M. Gao, Y. Zhang, A. Shi, R. A. Mariuzza and N. P. Weng (2017). “Sequence and Structural Analyses Reveal Distinct and Highly Diverse Human CD8 (+) TCR Repertoires to Immunodominant Viral Antigens.” Cell Rep 19 (3): 569-583.
Chiu, Y. L., J. P. Schneck and M. Oelke (2011). “HLA-Ig based artificial antigen presenting cells for efficient ex vivo expansion of human CTL.” J Vis Exp (50).
Cole, D. J., D. P. Weil, J. Shilyansky, M. Custer, Y. Kawakami, S. A. Rosenberg and M. I. Nishimura (1995). “Characterization of the functional specificity of a cloned T-cell receptor heterodimer recognizing the MART-1 melanoma antigen.” Cancer Res 55 (4): 748-752.
Collins, D. J., A. Neild. A. deMello, A. Q. Liu and Y. Ai (2015). “The Poisson distribution and beyond: methods for microfluidic droplet production and single cell encapsulation.” Lab Chip 15 (17): 3439-3459.
Corse, E., R. A. Gottschalk, M. Krogsgaard and J. P. Allison (2010). “Attenuated T cell responses to a high-potency ligand in vivo.” PLOS Biol 8 (9).
De Biasi, S., M. Meschiari, L. Gibellini, C. Bellinazzi, R. Borella, L. Fidanza, L. Gozzi, A. Iannone, D. Lo Tartaro, M. Mattioli, A. Paolini, M. Menozzi, J. Milic, G. Franceschi, R. Fantini, R. Tonelli, M. Sita, M. Sarti, T. Trenti, L. Brugioni, L. Cicchetti, F. Facchinetti, A. Pietrangelo, E. Clini, M. Girardis. G. Guaraldi, C. Mussini and A. Cossarizza (2020). “Marked T cell activation, senescence, exhaustion and skewing towards TH17 in patients with COVID-19 pneumonia.” Nat Commun 11 (1): 3434.
Dushek, O., M. Aleksic, R. J. Wheeler, H. Zhang, S. P. Cordoba, Y. C. Peng, J. L. Chen, V. Cerundolo, T. Dong, D. Coombs and P. A. van der Merwe (2011). “Antigen potency and maximal efficacy reveal a mechanism of efficient T cell activation.” Sci Signal 4 (176): ra39.
Fabregat, A., K. Sidiropoulos, G. Viteri, O. Forner, P. Marin-Garcia, V. Arnau, P. D′Eustachio, L. Stein and H. Hermjakob (2017). “Reactome pathway analysis: a high-performance in-memory approach.” BMC Bioinformatics 18 (1): 142.
Frith, M. C., N. F. Saunders, B. Kobe and T. L. Bailey (2008). “Discovering sequence motifs with arbitrary insertions and deletions.” PLOS Comput Biol 4 (4): e1000071.
Gocher, A. M., C. J. Workman and D. A. A. Vignali (2022). “Interferon-gamma: teammate or opponent in the tumour microenvironment?” Nat Rev Immunol 22 (3): 158-172.
Gong, Z., Q. Li, J. Shi and G. Ren (2022). “An Artifact in Intracellular Cytokine Staining for Studying T Cell Responses and Its Alleviation.” Front Immunol 13:759188.
Guy, C. S., K. M. Vignali, J. Temirov, M. L. Bettini, A. E. Overacre, M. Smeltzer, H. Zhang, J. B. Huppa, Y. H. Tsai, C. Lobry, J. Xie, P. J. Dempsey, H. C. Crawford, I. Aifantis, M. M. Davis and D. A. Vignali (2013). “Distinct TCR signaling pathways drive proliferation and cytokine production in T cells.” Nat Immunol 14 (3): 262-270.
Hao, Y., S. Hao, E. Andersen-Nissen, W. M. Mauck, 3rd, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming. B. Yeung, A. J. Rogers, J. M. McElrath, C. A. Blish, R. Gottardo, P. Smibert and R. Satija (2021). “Integrated analysis of multimodal single-cell data.” Cell 184 (13): 3573-3587 e3529.
Heintzman, D. R., E. L. Fisher and J. C. Rathmell (2022). “Microenvironmental influences on T cell immunity in cancer and inflammation.” Cell Mol Immunol 19 (3): 316-326.
Hogan, S. A., A. Courtier, P. F. Cheng, N. F. Jaberg-Bentele, S. M. Goldinger, M. Manuel, S. Perez, N. Plantier, J. F. Mouret, T. D. L. Nguyen-Kim, M. I. G. Raaijmakers, P. Kvistborg, N. Pasqual, J. Haanen, R. Dummer and M. P. Levesque (2019). “Peripheral Blood TCR Repertoire Profiling May Facilitate Patient Stratification for Immunotherapy against Melanoma.” Cancer Immunol Res 7 (1): 77-85.
Hu, Z., P. A. Ott and C. J. Wu (2018). “Towards personalized, tumour-specific, therapeutic vaccines for cancer.” Nat Rev Immunol 18 (3): 168-182.
Huse, M., B. F. Lillemeier, M. S. Kuhns, D. S. Chen and M. M. Davis (2006). “T cells use two directionally distinct pathways for cytokine secretion.” Nat Immunol 7 (3): 247-255.
Huth. A., X. Liang, S. Krebs, H. Blum and A. Moosmann (2019). “Antigen-Specific TCR Signatures of Cytomegalovirus Infection.” J Immunol 202 (3): 979-990.
Kalergis, A. M., N. Boucheron, M. A. Doucey, E. Palmieri, E. C. Goyarts, Z. Vegh, I. F. Luescher and S. G. Nathenson (2001). “Efficient T cell activation requires an optimal dwell-time of interaction between the TCR and the pMHC complex.” Nat Immunol 2 (3): 229-234.
Kanehisa, M. and S. Goto (2000). “KEGG: kyoto encyclopedia of genes and genomes.” Nucleic Acids Res 28 (1): 27-30.
Kim, J., S. Noh, J. A. Park, S. C. Park, S. J. Park. J. H. Lee, J. H. Ahn and T. Lee (2021). “Recent Advances in Aptasensor for Cytokine Detection: A Review.” Sensors (Basel) 21 (24).
Lever, M., P. K. Maini, P. A. van der Merwe and O. Dushek (2014). “Phenotypic models of T cell activation.” Nat Rev Immunol 14 (9): 619-629.
Li, H. M., T. Hiroi, Y. Zhang, A. Shi, G. Chen, S. De, E. J. Metter, W. H. Wood, 3rd, A. Sharov, J. D. Milner, K. G. Becker, M. Zhan and N. P. Weng (2016). “TCRbeta repertoire of CD4+ and CD8⁺ T cells is distinct in richness, distribution, and CDR3 amino acid composition.” J Leukoc Biol 99 (3): 505-513.
Limozin, L., M. Bridge, P. Bongrand, O. Dushek, P. A. van der Merwe and P. Robert (2019). “TCR-pMHC kinetics under force in a cell-free system show no intrinsic catch bond, but a minimal encounter duration before binding.” Proc Natl Acad Sci USA 116 (34): 16943-16948.
Lin, J. J. Y., S. T. Low-Nam, K. N. Alfieri, D. B. McAffee, N. C. Fay and J. T. Groves (2019). “Mapping the stochastic sequence of individual ligand-receptor binding events to cellular activation: T cells act on the rare events.” Sci Signal 12 (564).
Link, C. S., A. Eugster, F. Heidenreich, E. Rucker-Braun, M. Schmiedgen, U. Oelschlagel, D. Kuhn, S. Dietz, Y. Fuchs, A. Dahl, A. M. Domingues, C. Klesse, M. Schmitz, G. Ehninger, M. Bornhauser, J. Schetelig and E. Bonifacio (2016). “Abundant cytomegalovirus (CMV) reactive clonotypes in the CD8 (+) T cell receptor alpha repertoire following allogeneic transplantation.” Clin Exp Immunol 184 (3): 389-402.
Lyons, G. E., T. Moore, N. Brasic, M. Li, J. J. Roszkowski and M. I. Nishimura (2006). “Influence of human CD8 on antigen recognition by T-cell receptor-transduced cells.” Cancer Res 66 (23): 11455-11461.
Marofi, F., R. Motavalli, V. A. Safonov, L. Thangavelu, A. V. Yumashev, M. Alexander, N. Shomali, M. S. Chartrand, Y. Pathak, M. Jarahian, S. Izadi, A. Hassanzadeh, N. Shirafkan, S. Tahmasebi and F. M. Khiavi (2021). “CAR T cells in solid tumors: challenges and opportunities.” Stem Cell Res Ther 12 (1): 81.
Martens, M., A. Ammar, A. Riutta, A. Waagmeester, D. N. Slenter, K. Hanspers, A. M. R. D. Digles, E. N. Lopes, F. Ehrhart, L. J. Dupuis, L. A. Winckers, S. L. Coort, E. L. Willighagen, C. T. Evelo, A. R. Pico and M. Kutmon (2021). “WikiPathways: connecting communities.” Nucleic Acids Res 49 (D1): D613-D621.
Mazet, J. M., J. N. Mahale, O. Tong, R. A. Watson, A. V. Lechuga-Vieco, G. Pirgova, V. W. C. Lau, M. Attar, L. A. Koneva, S. N. Sansom, B. P. Fairfax and A. Gerard (2023). “IFNgamma signaling in cytotoxic T cells restricts anti-tumor responses by inhibiting the maintenance and diversity of intra-tumoral stem-like T cells.” Nat Commun 14 (1): 321.
McLane, L. M., M. S. Abdel-Hakeem and E. J. Wherry (2019). “CD8 T Cell Exhaustion During Chronic Viral Infection and Cancer.” Annu Rev Immunol 37:457-495.
Nakasone, H., M. Kusuda, K. Terasako-Saito, K. Kawamura, Y. Akahoshi, M. Kawamura, J. Takeshita, S. Kawamura, N. Yoshino, K. Yoshimura, Y. Misaki, A. Gomyo, K. Kameda, M. Tamaki, A. Tanihara, S. I. Kimura, S. Kako and Y. Kanda (2021). “Features of repertoire diversity and gene expression in human cytotoxic T cells following allogeneic hematopoietic cell transplantation.” Commun Biol 4 (1): 1177.
Neal, L. R., S. R. Bailey, M. M. Wyatt, J. S. Bowers, K. Majchrzak, M. H. Nelson, C. Haupt, C. M. Paulos and J. C. Varela (2017). “The Basics of Artificial Antigen Presenting Cells in T Cell-Based Cancer Immunotherapies.” J Immunol Res Ther 2 (1): 68-79.
Nesterenko, P. A., J. McLaughlin, D. Cheng, N. J. Bangayan, G. Burton Sojo, C. S. Seet, Y. Qin, Z. Mao, M. B. Obusan, J. W. Phillips and O. N. Witte (2021). “Droplet-based mRNA sequencing of fixed and permeabilized cells by CLInt-seq allows for antigen-specific TCR cloning.” Proc Natl Acad Sci USA 118 (3).
Nicolet, B. P., A. Guislain and M. C. Wolkers (2017). “Combined Single-Cell Measurement of Cytokine mRNA and Protein Identifies T Cells with Persistent Effector Function.” J Immunol 198 (2): 962-970.
Peng, F., L. K. Mansson, S. H. Holm, S. Ghosh, G. Carlstrom, J. J. Crassous, P. Schurtenberger and J. O. Tegenfeldt (2019). “A Droplet-Based Microfluidics Route to Temperature-Responsive Colloidal Molecules.” J Phys Chem B 123 (43): 9260-9271.
Penkava, F., M. D. C. Velasco-Herrera, M. D. Young, N. Yager, L. N. Nwosu, A. G. Pratt, A. L. Lara, C. Guzzo, A. Maroof, L. Mamanova, S. Cole, M. Efremova, D. Simone, A. Filer, C. C. Brown, A. L. Croxford, J. D. Isaacs, S. Teichmann, P. Bowness, S. Behjati and M. Hussein A1-Mossawi (2020). “Single-cell sequencing reveals clonal expansions of pro-inflammatory synovial CD8 T cells expressing tissue-homing receptors in psoriatic arthritis.” Nat Commun 11 (1): 4767.
Qi, Q., Y. Liu, Y. Cheng, J. Glanville, D. Zhang, J. Y. Lee, R. A. Olshen, C. M. Weyand, S. D. Boyd and J. J. Goronzy (2014). “Diversity and clonal selection in the human T-cell repertoire.” Proc Natl Acad Sci USA 111 (36): 13139-13144.
Qiu, L., F. Wimmers, J. Weiden, H. A. Heus, J. Tel and C. G. Figdor (2017). “A membrane-anchored aptamer sensor for probing IFNgamma secretion by single cells.” Chem Commun (Camb) 53 (57): 8066-8069.
Rius, C., M. Attaf, K. Tungatt, V. Bianchi, M. Legut, A. Bovay, M. Donia, P. Thor Straten, M. Peakman, I. M. Svane, S. Ott, T. Connor, B. Szomolay, G. Dolton and A. K. Sewell (2018). “Peptide-MHC Class I Tetramers Can Fail To Detect Relevant Functional T Cell Clonotypes and Underestimate Antigen-Reactive T Cell Populations.” J Immunol 200 (7): 2263-2279.
Robins, H. S., S. K. Srivastava, P. V. Campregher, C. J. Turtle, J. Andriesen, S. R. Riddell, C. S. Carlson and E. H. Warren (2010). “Overlap and effective size of the human CD8⁺ T cell receptor repertoire.” Sci Transl Med 2 (47): 47ra64.
Schaefer, C. F., K. Anthony, S. Krupa, J. Buchoff, M. Day, T. Hannay and K. H. Buetow (2009). “PID: the Pathway Interaction Database.” Nucleic Acids Res 37 (Database issue): D674-679.
Shah, K., A. A1-Haidari, J. Sun and J. U. Kazi (2021). “T cell receptor (TCR) signaling in health and disease.” Signal Transduct Target Ther 6 (1): 412.
Sharma, P. and J. P. Allison (2015). “Immune checkpoint targeting in cancer therapy: toward combination strategies with curative potential.” Cell 161 (2): 205-214.
Sheu, B. C., R. H. Lin, H. N. Ho and S. C. Huang (1997). “Down-regulation of CD25 expression on the surface of activated tumor-infiltrating lymphocytes in human cervical carcinoma.” Hum Immunol 56 (1-2): 39-48.
Shyer, J. A., R. A. Flavell and W. Bailis (2020). “Metabolic signaling in T cells.” Cell Res 30 (8): 649-659.
Stone, J. D. and D. M. Kranz (2013). “Role of T cell receptor affinity in the efficacy and specificity of adoptive T cell therapies.” Front Immunol 4:244.
Subramanian, A., P. Tamayo, V. K. Mootha, S. Mukherjee, B. L. Ebert, M. A. Gillette, A. Paulovich, S. L. Pomeroy, T. R. Golub, E. S. Lander and J. P. Mesirov (2005). “Gene set enrichment analysis: a knowledge-based approach for interpreting genome-wide expression profiles.” Proc Natl Acad Sci USA 102 (43): 15545-15550.
Tang, H., M. Zhu, J. Qiao and Y. X. Fu (2017). “Lymphotoxin signalling in tertiary lymphoid structures and immunotherapy.” Cell Mol Immunol 14 (10): 809-818.
Tsai, H. F., A. Trubelja, A. Q. Shen and G. Bao (2017). “Tumour-on-a-chip: microfluidic models of tumour morphology, growth and microenvironment.” J R Soc Interface 14 (131).
Tsimberidou, A. M., K. Van Morris, H. H. Vo, S. Eck, Y. F. Lin, J. M. Rivas and B. S. Andersson (2021). “T-cell receptor-based therapy: an innovative therapeutic approach for solid tumors.” J Hematol Oncol 14 (1): 102.
Valpione, S., P. A. Mundra, E. Galvani, L. G. Campana, P. Lorigan, F. De Rosa, A. Gupta, J. Weightman, S. Mills, N. Dhomen and R. Marais (2021). “The T cell receptor repertoire of tumor infiltrating T cells is predictive and prognostic for cancer survival.” Nat Commun 12 (1): 4098.
van Loo, G. and M. J. M. Bertrand (2023). “Death by TNF: a road to inflammation.” Nat Rev Immunol 23 (5): 289-303.
Wang, G. C., P. Dash, J. A. McCullers, P. C. Doherty and P. G. Thomas (2012). “T cell receptor alphabeta diversity inversely correlates with pathogen-specific antibody levels in human cytomegalovirus infection.” Sci Transl Med 4 (128): 128ra142.
Wedekind, M. F., N. L. Denton, C. Y. Chen and T. P. Cripe (2018). “Pediatric Cancer Immunotherapy: Opportunities and Challenges.” Paediatr Drugs 20 (5): 395-408.
Wherry. E. J. and M. Kurachi (2015). “Molecular and cellular insights into T cell exhaustion.” Nat Rev Immunol 15 (8): 486-499.
Yarmarkovich, M., Q. F. Marshall, J. M. Warrington, R. Premaratne, A. Farrel, D. Groff, W. Li, M. di Marco, E. Runbeck, H. Truong, J. S. Toor, S. Tripathi, S. Nguyen, H. Shen, T. Noel, N. L. Church, A. Weiner, N. Kendsersky, D. Martinez, R. Weisberg, M. Christie, L. Eisenlohr, K. R. Bosse, D. S. Dimitrov, S. Stevanovic, N. G. Sgourakis, B. R. Kiefel and J. M. Maris (2021). “Cross-HLA targeting of intracellular oncoproteins with peptide-centric CARs.” Nature 599 (7885): 477-484.
You, M., Y. Lyu, D. Han, L. Qiu, Q. Liu, T. Chen, C. Sam Wu, L. Peng, L. Zhang, G. Bao and W. Tan (2017). “DNA probes for monitoring dynamic and transient molecular encounters on live cell membranes.” Nat Nanotechnol 12 (5): 453-459.
Yuan, Y., J. Brouchon, J. M. Calvo-Calle, J. Xia, L. Sun, X. Zhang, K. L. Clayton, F. Ye, D. A. Weitz and J. A. Heyman (2020). “Droplet encapsulation improves accuracy of immune cell cytokine capture assays.” Lab Chip 20 (8): 1513-1520.
Zhang, Z., D. Xiong, X. Wang. H. Liu and T. Wang (2021). “Mapping the functional landscape of T cell receptor repertoires by single-T cell transcriptomics.” Nat Methods 18 (1): 92-99.
Zheng, G. X., J. M. Terry, P. Belgrader, P. Ryvkin, Z. W. Bent, R. Wilson, S. B. Ziraldo, T. D. Wheeler, G. P. McDermott, J. Zhu, M. T. Gregory, J. Shuga, L. Montesclaros, J. G. Underwood, D. A. Masquelier, S. Y. Nishimura, M. Schnall-Levin, P. W. Wyatt, C. M. Hindson, R. Bharadwaj, A. Wong, K. D. Ness, L. W. Beppu, H. J. Deeg, C. McFarland, K. R. Loeb, W. J. Valente, N. G. Ericson, E. A. Stevens, J. P. Radich, T. S. Mikkelsen, B. J. Hindson and J. H. Bielas (2017). “Massively parallel digital transcriptional profiling of single cells.” Nat Commun 8:14049.

1. A method detecting T Cell Receptor (TCR) activation on a target T cell comprising:
(a) providing a single T cell decorated with a cytokine-specific detectable aptamer beacon with a modification that allows said cytokine-specific detectable aptamer beacon to be immobilized to the T cell's surface;
(b) providing one or more antigen-loaded artificial antigen presenting cells (aAPC);
(c) co-encapsulating said T cell and said one or more aAPCs in a microdroplet;
(d) incubating said microdroplet for a time sufficient to permit T cell activation by said aAPC;
(e) sorting and extracting an activated T cell, wherein said activated T cell is sorted by fluorescence activated cell sorting based on activation and detection of said cytokine-specific detectable label; and
(f) sequencing TCR sequences from the sorted and extracted T cell of step (e).

2. The method of claim 1, wherein multiple genetically distinct single T cells in distinct microdroplets are processed together.

3. The method of claim 1, wherein steps (c) and (d) are performed on a microfluidic chip, such as a PDM or PMMA chip.

4. The method of claim 1, wherein the microdroplet is generated through a T-junction configuration or flow-focusing configuration.

5. The method of claim 1, wherein step (d) comprises incubation at about 35-39° C. for about 24-72 hours.

6. The method of claim 1, wherein said cytokine-specific detectable aptamer beacon is a single-stranded DNA (ssDNA) or an RNA oligonucleotide sequence that binds to its target cytokine with high specificity and affinity, such as nucleic acid aptamer with a fluorescent label that is quenched prior to binding to the cytokine for which the aptamer is specific.

7. The method of claim 1, wherein the cytokine for which the cytokine-specific detectable aptamer beacon is specific is selected from IFN-γ, TNF-α and IL-6.

8. The method of claim 1, wherein step (f) comprises a single-cell sequencing protocol, such as 10× Genomics protocol, inDrop protocol and SMART-seq protocol.

9. The method of claim 1, wherein said microdroplet is a 100-200 μm diameter water-in-oil droplet.

10. The method of claim 1, wherein the aAPC is a xenogeneic cell (such as K562-aAPC), PLGA microparticle, sepharose microparticle, polystyrene microparticle, liposome and nanoparticle.

11. The method of claim 1, wherein said modification is streptavidin-aptamer conjugate bound to a cell surface amino group through NHS-biotin crosslinker or a lipophilic residue (cholesterol, tocopherol, C18 chains, diacyl phospholipid, etc.) modified aptamer displayed on cell surface by hydrophobic insertion.

12. The method of claim 1, further comprising cloning a TCR gene from said T cell.

13. The method of claim 12, further comprising transforming a target cell with said TCR, such as with a non-viral or viral (e.g., lentiviral) vectors.

14. The method of claim 13, wherein said target cell is a T cell circulating in patients' peripheral blood

15. A T Cell Receptor identified according to a method of claim 1.


SEQUENCE LISTING (117 sequences)

SEQ ID NO: 1 - DNA, 906 bp, Homo sapiens
  source 1..906: mol_type = other DNA
ccctcactccttctctaggcgccggaattcagatctaccatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagttgtgcagcaagtaatacctatgggaacaacagactcgcttttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 2 - DNA, 987 bp, Homo sapiens
  source 1..987: mol_type = other DNA
gcaggagacgtggaagaaaaccccggtcccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgtgcgccagcagcctaaaacgggggctgacagatacgcagtattttgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 3 - DNA, 894 bp, Homo sapiens
  source 1..894: mol_type = other DNA
ccctcactccttctctaggcgccggaattcagatctaccatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagatgtgctgtccattggatggatagcagctataaattgatcttcaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 4 - DNA, 987 bp, Homo sapiens
  source 1..987: mol_type = other DNA
gcaggagacgtggaagaaaaccccggtcccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacagttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatttgtgtgccaccagtgaccggcaggggtacaatgagcagttcttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 5 - DNA, 921 bp, Homo sapiens
  source 1..921: mol_type = other DNA
ccctcactccttctctaggcgccggaattcagatctaccatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagctgtgcttataggccctcttattcaggaaacacacctcttgtctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 6 - DNA, 984 bp, Homo sapiens
  source 1..984: mol_type = other DNA
gcaggagacgtggaagaaaaccccggtcccatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaatgcgccagcagcctacggaccaatacctacgagcagtacttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 7 - DNA, 915 bp, Homo sapiens
  source 1..915: mol_type = other DNA
ccctcactccttctctaggcgccggaattcagatctaccatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttttgtgcctcccagggcactaactttggaaatgagaaattaacctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 8 - DNA, 978 bp, Homo sapiens
  source 1..978: mol_type = other DNA
gcaggagacgtggaagaaaaccccggtcccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgtgccagcagtttatatgggggaaacactgaagctttctttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 9 - DNA, 915 bp, Homo sapiens
  source 1..915: mol_type = other DNA
ccctcactccttctctaggcgccggaattcagatctaccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagactgtgctgtgagagacgaaaattcaggatacagcaccctcacctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 10 - DNA, 984 bp, Homo sapiens
  source 1..984: mol_type = other DNA
gcaggagacgtggaagaaaaccccggtcccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttatgtgccagcagcttaaaggacagctataatgaaaaactgttttttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 11 - DNA, 909 bp, Homo sapiens
  source 1..909: mol_type = other DNA
ccctcactccttctctaggcgccggaattcagatctaccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagactgtgctgtgttcccctctaacgactacaagctcagctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 12 - DNA, 987 bp, Homo sapiens
  source 1..987: mol_type = other DNA
gcaggagacgtggaagaaaaccccggtcccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacagttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatttgtgtgccaccagtgatccggggagggcttacaatcagccccagcattttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 13 - DNA, 897 bp, Homo sapiens
  source 1..897: mol_type = other DNA
ccctcactccttctctaggcgccggaattcagatctaccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggactgtgcctacgtcagagatgacaagatcatctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 14 - DNA, 984 bp, Homo sapiens
  source 1..984: mol_type = other DNA
gcaggagacgtggaagaaaaccccggtcccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttatgtgccagcagtttagcgtcaggggggaattcacccctccactttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 15 - DNA, 906 bp, Homo sapiens
  source 1..906: mol_type = other DNA
ccctcactccttctctaggcgccggaattcagatctaccatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttttgtgccttcccttacaataacaatgacatgcgctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 16 - DNA, 975 bp, Homo sapiens
  source 1..975: mol_type = other DNA
gcaggagacgtggaagaaaaccccggtcccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgtgccagcagtttagagggttacactgaagctttctttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 17 - DNA, 897 bp, Homo sapiens
  source 1..897: mol_type = other DNA
ccctcactccttctctaggcgccggaattcagatctaccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggactgtgccctgaacagagatgacaagatcatctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 18 - DNA, 993 bp, Homo sapiens
  source 1..993: mol_type = other DNA
gcaggagacgtggaagaaaaccccggtcccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagcttatgtgccagcagcttagccgggacagggccgaccggggagctgttttttgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 19 - DNA, 912 bp, Homo sapiens
  source 1..912: mol_type = other DNA
ccctcactccttctctaggcgccggaattcagatctaccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagttgtgctgtgagtgtcacgggaaggcagggaggaaagcttatcttcaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 20 - DNA, 990 bp, Homo sapiens
  source 1..990: mol_type = other DNA
gcaggagacgtggaagaaaaccccggtcccatgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaatgtgccagcagccaagcacagggggcgagtcggagcaatcagccccagcattttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 21 - DNA, 900 bp, Homo sapiens
  source 1..900: mol_type = other DNA
ccctcactccttctctaggcgccggaattcagatctaccatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttttgtgcccgaaacaccggtaaccagttctattttatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 22 - DNA, 981 bp, Homo sapiens
  source 1..981: mol_type = other DNA
gcaggagacgtggaagaaaaccccggtcccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactgtgccagcagttaccagacaggggctagctatggctacaccttcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 23 - DNA, 915 bp, Homo sapiens
  source 1..915: mol_type = other DNA
ccctcactccttctctaggcgccggaattcagatctaccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggttgtgctgtgggtgaggattatggtggtgctacaaacaagctcatctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 24 - DNA, 990 bp, Homo sapiens
  source 1..990: mol_type = other DNA
gcaggagacgtggaagaaaaccccggtcccatgggtcctgggcttctccactggatggccctttgtctccttggaacaggtcatggggatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacacagccatgtacctgtgtgccaccagcagatgtgccaccagcaggatcggacccctccaggcctacgagcagtacttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 25 - DNA, 903 bp, Homo sapiens
  source 1..903: mol_type = other DNA
ccctcactccttctctaggcgccggaattcagatctaccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaattgtgcagagaaccaggcaggaactgctctgatctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 26 - DNA, 978 bp, Homo sapiens
  source 1..978: mol_type = other DNA
gcaggagacgtggaagaaaaccccggtcccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgtgccagcagcgctcagggggtggtgactgaagctttctttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 27 - DNA, 909 bp, Homo sapiens
  source 1..909: mol_type = other DNA
ccctcactccttctctaggcgccggaattcagatctaccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggtgtgctgtgaaagaagtggagggcggatctgaaaagctggtctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 28 - DNA, 981 bp, Homo sapiens
  source 1..981: mol_type = other DNA
gcaggagacgtggaagaaaaccccggtcccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgtgccagcagtgtgacagggttctgggagcagtacttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 29 - DNA, 906 bp, Homo sapiens
  source 1..906: mol_type = other DNA
ccctcactccttctctaggcgccggaattcagatctaccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgagttgtgctgtgagtgatcaaggggactacaagctcagctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 30 - DNA, 990 bp, Homo sapiens
  source 1..990: mol_type = other DNA
gcaggagacgtggaagaaaaccccggtcccatggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccatgtgccagctcacctcgggggggccggatccacgagcagtacttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 31 - DNA, 909 bp, Homo sapiens
  source 1..909: mol_type = other DNA
ccctcactccttctctaggcgccggaattcagatctaccatggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggtgtgctgtgagttttggctctagcaacacaggcaaactaatctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 32 - DNA, 1002 bp, Homo sapiens
  source 1..1002: mol_type = other DNA
gcaggagacgtggaagaaaaccccggtcccatgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaatgcgccagcagccaagataattctagggcagcctacaatgagcagttcttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 33 - DNA, 903 bp, Homo sapiens
  source 1..903: mol_type = other DNA
ccctcactccttctctaggcgccggaattcagatctaccatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaattgtgcagagtcctcttccgggtatgcactcaacttcaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 34 - DNA, 978 bp, Homo sapiens
  source 1..978: mol_type = other DNA
gcaggagacgtggaagaaaaccccggtcccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatgtgccagcagttcacagggggctcgaactgaagctttctttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 35 - DNA, 915 bp, Homo sapiens
  source 1..915: mol_type = other DNA
ccctcactccttctctaggcgccggaattcagatctaccatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagctgtgctgctgaggtcaattccgggtatgcactcaacttcaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 36 - DNA, 978 bp, Homo sapiens
  source 1..978: mol_type = other DNA
gcaggagacgtggaagaaaaccccggtcccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttatgtgccagcagcgtacgagagacccagtacttcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctgactcgagcgggatcaattccgccccccccct

SEQ ID NO: 37 - DNA, 891 bp, Homo sapiens
  source 1..891: mol_type = other DNA
ccctcactccttctctaggcgccggaattcagatctaccatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggagtgtgctgggggaggtgctgacggactcacctttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggctccggagccacgaacttctctctgttaaagcaagcaggagacgtggaagaaaaccccggtccc

SEQ ID NO: 38 - DNA, 975 bp, Homo sapiens
  source 1..975: mol_type = other DNA
gcaggagacgtggaagaaaaccccggtcccatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaatgcagcgttgtaccccgggacagggtagctggaaacaccatatattttgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgactcgagcgggatcaattccgccccccccct

SEQ ID NO: 39 - protein, 14 aa, Homo sapiens
CAASNTYGNNRLAF

SEQ ID NO: 40 - protein, 14 aa, Homo sapiens
CAVHWMDSSYKLIF

SEQ ID NO: 41 - protein, 15 aa, Homo sapiens
CAYRPSYSGNTPLVF

SEQ ID NO: 42 - protein, 15 aa, Homo sapiens
CASQGTNFGNEKLTF

SEQ ID NO: 43 - protein, 15 aa, Homo sapiens
CAVRDENSGYSTLTF

SEQ ID NO: 44 - protein, 13 aa, Homo sapiens
CAVFPSNDYKLSF

SEQ ID NO: 45 - protein, 11 aa, Homo sapiens
CAYVRDDKIIF

SEQ ID NO: 46 - protein, 12 aa, Homo sapiens
CAFPYNNNDMRF

SEQ ID NO: 47 - protein, 11 aa, Homo sapiens
CALNRDDKIIF

SEQ ID NO: 48 - protein, 15 aa, Homo sapiens
CAVSVTGRQGGKLIF

SEQ ID NO: 49 - protein, 11 aa, Homo sapiens
CARNTGNQFYF

SEQ ID NO: 50 - protein, 16 aa, Homo sapiens
CAVGEDYGGATNKLIF

SEQ ID NO: 51 - protein, 12 aa, Homo sapiens
CAENQAGTALIF

SEQ ID NO: 52 - protein, 15 aa, Homo sapiens
CAVKEVEGGSEKLVF

SEQ ID NO: 53 - protein, 13 aa, Homo sapiens
CAVSDQGDYKLSF

SEQ ID NO: 54 - protein, 15 aa, Homo sapiens
CAVSFGSSNTGKLIF

SEQ ID NO: 55 - protein, 12 aa, Homo sapiens
CAESSSGYALNF

SEQ ID NO: 56 - protein, 13 aa, Homo sapiens
CAAEVNSGYALNF

SEQ ID NO: 57 - protein, 11 aa, Homo sapiens
CAGGGADGLTF

SEQ ID NO: 58 - DNA, 42 bp, Homo sapiens
  source 1..42: mol_type = other DNA
tgtgcagcaagtaatacctatgggaacaacagactcgctttt

SEQ ID NO: 59 - DNA, 42 bp, Homo sapiens
  source 1..42: mol_type = other DNA
tgtgctgtccattggatggatagcagctataaattgatcttc

SEQ ID NO: 60 - DNA, 45 bp, Homo sapiens
  source 1..45: mol_type = other DNA
tgtgcttataggccctcttattcaggaaacacacctcttgtcttt

SEQ ID NO: 61 - DNA, 45 bp, Homo sapiens
  source 1..45: mol_type = other DNA
tgtgcctcccagggcactaactttggaaatgagaaattaaccttt

SEQ ID NO: 62 - DNA, 45 bp, Homo sapiens
  source 1..45: mol_type = other DNA
tgtgctgtgagagacgaaaattcaggatacagcaccctcaccttt

SEQ ID NO: 63 - DNA, 39 bp, Homo sapiens
  source 1..39: mol_type = other DNA
tgtgctgtgttcccctctaacgactacaagctcagcttt

SEQ ID NO: 64 - DNA, 33 bp, Homo sapiens
  source 1..33: mol_type = other DNA
tgtgcctacgtcagagatgacaagatcatcttt

SEQ ID NO: 65 - DNA, 36 bp, Homo sapiens
  source 1..36: mol_type = other DNA
tgtgccttcccttacaataacaatgacatgcgcttt

SEQ ID NO: 66 - DNA, 33 bp, Homo sapiens
  source 1..33: mol_type = other DNA
tgtgccctgaacagagatgacaagatcatcttt

SEQ ID NO: 67 - DNA, 45 bp, Homo sapiens
  source 1..45: mol_type = other DNA
tgtgctgtgagtgtcacgggaaggcagggaggaaagcttatcttc

SEQ ID NO: 68 - DNA, 33 bp, Homo sapiens
  source 1..33: mol_type = other DNA
tgtgcccgaaacaccggtaaccagttctatttt

SEQ ID NO: 69 - DNA, 48 bp, Homo sapiens
  source 1..48: mol_type = other DNA
tgtgctgtgggtgaggattatggtggtgctacaaacaagctcatcttt

SEQ ID NO: 70 - DNA, 36 bp, Homo sapiens
  source 1..36: mol_type = other DNA
tgtgcagagaaccaggcaggaactgctctgatcttt

SEQ ID NO: 71 - DNA, 45 bp, Homo sapiens
  source 1..45: mol_type = other DNA
tgtgctgtgaaagaagtggagggcggatctgaaaagctggtcttt

SEQ ID NO: 72 - DNA, 39 bp, Homo sapiens
  source 1..39: mol_type = other DNA
tgtgctgtgagtgatcaaggggactacaagctcagcttt

SEQ ID NO: 73 - DNA, 45 bp, Homo sapiens
  source 1..45: mol_type = other DNA
tgtgctgtgagttttggctctagcaacacaggcaaactaatcttt

SEQ ID NO: 74 - DNA, 36 bp, Homo sapiens
  source 1..36: mol_type = other DNA
tgtgcagagtcctcttccgggtatgcactcaacttc

SEQ ID NO: 75 - DNA, 39 bp, Homo sapiens
  source 1..39: mol_type = other DNA
tgtgctgctgaggtcaattccgggtatgcactcaacttc

SEQ ID NO: 76 - DNA, 33 bp, Homo sapiens
  source 1..33: mol_type = other DNA
tgtgctgggggaggtgctgacggactcaccttt

SEQ ID NO: 77 - protein, 15 aa, Homo sapiens
CASSLKRGLTDTQYF

SEQ ID NO: 78 - protein, 14 aa, Homo sapiens
CATSDRQGYNEQFF

SEQ ID NO: 79 - protein, 14 aa, Homo sapiens
CASSLRTNTYEQYF

SEQ ID NO: 80 - protein, 14 aa, Homo sapiens
CASSLYGGNTEAFF

SEQ ID NO: 81 - protein, 15 aa, Homo sapiens
CASSLKDSYNEKLFF

SEQ ID NO: 82 - protein, 16 aa, Homo sapiens
CATSDPGRAYNQPQHF

SEQ ID NO: 83 - protein, 15 aa, Homo sapiens
CASSLASGGNSPLHF

SEQ ID NO: 84 - protein, 13 aa, Homo sapiens
CASSLEGYTEAFF

SEQ ID NO: 85 - protein, 16 aa, Homo sapiens
CASSLAGTGPTGELFF

SEQ ID NO: 86 - protein, 18 aa, Homo sapiens
CASSQAQGASRSNQPQHF

SEQ ID NO: 87 - protein, 15 aa, Homo sapiens
CASSYQTGASYGYTF

SEQ ID NO: 88 - protein, 16 aa, Homo sapiens
CATSRIGPLQAYEQYF

SEQ ID NO: 89 - protein, 14 aa, Homo sapiens
CASSAQGVVTEAFF

SEQ ID NO: 90 - protein, 13 aa, Homo sapiens
CASSVTGFWEQYF

SEQ ID NO: 91 - protein, 15 aa, Homo sapiens
CASSPRGGRIHEQYF

SEQ ID NO: 92 - protein, 17 aa, Homo sapiens
CASSQDNSRAAYNEQFF

SEQ ID NO: 93 - protein, 14 aa, Homo sapiens
CASSSQGARTEAFF

SEQ ID NO: 94 - protein, 11 aa, Homo sapiens
CASSVRETQYF

SEQ ID NO: 95 - protein, 16 aa, Homo sapiens
CSVVPRDRVAGNTIYF

SEQ ID NO: 96 - DNA, 45 bp, Homo sapiens
  source 1..45: mol_type = other DNA
tgcgccagcagcctaaaacgggggctgacagatacgcagtatttt

SEQ ID NO: 97 - DNA, 42 bp, Homo sapiens
  source 1..42: mol_type = other DNA
tgtgccaccagtgaccggcaggggtacaatgagcagttcttc

SEQ ID NO: 98 - DNA, 42 bp, Homo sapiens
  source 1..42: mol_type = other DNA
tgcgccagcagcctacggaccaatacctacgagcagtacttc

SEQ ID NO: 99 - DNA, 42 bp, Homo sapiens
  source 1..42: mol_type = other DNA
tgtgccagcagtttatatgggggaaacactgaagctttcttt

SEQ ID NO: 100 - DNA, 45 bp, Homo sapiens
  source 1..45: mol_type = other DNA
tgtgccagcagcttaaaggacagctataatgaaaaactgtttttt

SEQ ID NO: 101 - DNA, 48 bp, Homo sapiens
  source 1..48: mol_type = other DNA
tgtgccaccagtgatccggggagggcttacaatcagccccagcatttt

SEQ ID NO: 102 - DNA, 45 bp, Homo sapiens
  source 1..45: mol_type = other DNA
tgtgccagcagtttagcgtcaggggggaattcacccctccacttt

SEQ ID NO: 103 - DNA, 39 bp, Homo sapiens
  source 1..39: mol_type = other DNA
tgtgccagcagtttagagggttacactgaagctttcttt

SEQ ID NO: 104 - DNA, 48 bp, Homo sapiens
  source 1..48: mol_type = other DNA
tgtgccagcagcttagccgggacagggccgaccggggagctgtttttt

SEQ ID NO: 105 - DNA, 54 bp, Homo sapiens
  source 1..54: mol_type = other DNA
tgtgccagcagccaagcacagggggcgagtcggagcaatcagccccagcatttt

SEQ ID NO: 106 - DNA, 45 bp, Homo sapiens
  source 1..45: mol_type = other DNA
tgtgccagcagttaccagacaggggctagctatggctacaccttc

SEQ ID NO: 107 - DNA, 48 bp, Homo sapiens
  source 1..48: mol_type = other DNA
tgtgccaccagcaggatcggacccctccaggcctacgagcagtacttc

SEQ ID NO: 108 - DNA, 42 bp, Homo sapiens
  source 1..42: mol_type = other DNA
tgtgccagcagcgctcagggggtggtgactgaagctttcttt

SEQ ID NO: 109 - DNA, 39 bp, Homo sapiens
  source 1..39: mol_type = other DNA
tgtgccagcagtgtgacagggttctgggagcagtacttc

SEQ ID NO: 110 - DNA, 45 bp, Homo sapiens
  source 1..45: mol_type = other DNA
tgtgccagctcacctcgggggggccggatccacgagcagtacttc

SEQ ID NO: 111 - DNA, 51 bp, Homo sapiens
  source 1..51: mol_type = other DNA
tgcgccagcagccaagataattctagggcagcctacaatgagcagttcttc

SEQ ID NO: 112 - DNA, 42 bp, Homo sapiens
  source 1..42: mol_type = other DNA
tgtgccagcagttcacagggggctcgaactgaagctttcttt

SEQ ID NO: 113 - DNA, 33 bp, Homo sapiens
  source 1..33: mol_type = other DNA
tgtgccagcagcgtacgagagacccagtacttc

SEQ ID NO: 114 - DNA, 48 bp, Homo sapiens
  source 1..48: mol_type = other DNA
tgcagcgttgtaccccgggacagggtagctggaaacaccatatatttt

SEQ ID NO: 115 - DNA, 36 bp, synthetic construct
  source 1..36: mol_type = other DNA
aggggttggttgtgttgggtgttgtgtccaacccct

SEQ ID NO: 116 - DNA, 36 bp, synthetic construct
  source 1..36: mol_type = other DNA
aggggttggacacaacacccaacacaaccaacccct

SEQ ID NO: 117 - protein, 9 aa, synthetic construct
NLVPMVATV